(19) United States
(12) Patent Application Publication
(10) Pub. No.: US 2026/0015592 A1
(43) Pub. Date: Jan. 15, 2026

(54) NEDDYLATION-ACTIVATING ENZYME INHIBITORS AS VIRAL SENSITIZERS AND USES THEREOF

(71) Applicant: Ottawa Hospital Research Institute, Ottawa (CA)

(72) Inventors: Jean-Simon Diallo, Ottawa (CA); Boaz Wong, Concord (CA); Anabel Bergeron, Orleans (CA); Rozanne Arulanandam, Orleans (CA); Glib Viktorovich Maznyi, Ottawa (CA)

(73) Assignee: Ottawa Hospital Research Institute, Ottawa, ON (CA)

(21) Appl. No.: 18/993,043

(22) PCT Filed: Jul. 14, 2023
(86) PCT No.: PCT/CA2023/050955

§ 371 Date: Jan. 10, 2025

Related U.S. Application Data
(60) Provisional application No. 63/389,064, filed on Jul. 14, 2022.

(51) Int. Cl. C12N 7/02; A61K 39/00; A61K 39/145; A61K 39/205; A61K 45/06; A61K 48/00; A61P 35/00; C12N 15/86

(52) U.S. Cl. CPC C12N 7/025; A61K 39/0011; A61K 39/145; A61K 39/205; A61K 45/06; A61K 48/005; A61P 35/00; C12N 15/86; C12N 2710/10043; C12N 2710/10134; C12N 2710/16634; C12N 2710/16643; C12N 2740/15034; C12N 2740/15043; C12N 2750/14134; C12N 2750/14143; C12N 2760/16034; C12N 2760/16043; C12N 2760/20034; C12N 2760/20043; C12N 2770/36034; C12N 2770/36043

(57) ABSTRACT

The present application relates to viral sensitizers. More specifically, the present application relates to neddylation-activating enzyme inhibitors, as well as processes for their preparation and methods of using such compounds and compositions as viral sensitizers. The present application includes a method of increasing permissiveness of a cell to a virus or genetic material encoding components of the virus, comprising administering an effective amount of a neddylation-activating enzyme (NAE) inhibitor, or a salt, solvate and/or prodrug thereof, to the cell.

### CROSS-REFERENCE TO RELATED APPLICATION

This application claims the benefit of priority to U.S. Provisional Application No. 63/389,064, filed Jul. 14, 2022, the contents of which are incorporated herein by reference in their entirety.

### FIELD

The present application relates to viral sensitizers. More specifically, the present application relates to neddylation-activating enzyme inhibitors, as well as processes for their preparation and methods of using such compounds and compositions as viral sensitizers.

### BACKGROUND

Viruses have been long recognized as effective delivery platforms for therapeutic regimens across many diseases. Firstly, viral gene therapy is the most common delivery vehicle to re-introduce critical gene products in monogenic loss-of-function diseases such as cystic fibrosis. Viral delivery of gene products to restore physiological processes have also since been used with success. Secondly, the inherent anti-tumour properties of viruses have given rise to the field of oncolytic virotherapy, an established immunotherapy for the treatment of cancer. Unfortunately, both therapeutic avenues suffer from finite viral infection/replicating capacity, thus limiting its therapeutic efficacy output. Finally, outside of the direct treatment of disease, virus production remains a crucial chokepoint that limits virus-based therapeutic development. The antiviral defenses of manufacturing cells can represent a rate-determining step in the manufacturing process, limiting the final quantity of viral product. Finding methods to improve the replicating yield of these viral vectors would prove valuable for improving the therapeutic efficacy viral-based therapeutics and increasing cost-efficiency of viral-based manufacturing.

Viral sensitizing compounds are compounds that increase the susceptibility of cells to infection by a virus (or to transduction of a cell by genetic material encoding viral-like nucleic acids) by suppressing the cell's innate antiviral programmes. Such compounds may increase production of viral vectors from a cell, or they may lead to increased infection or transfection efficiency (where non-replicating viruses or genetic material are employed).

Several compounds with viral sensitizing properties have been previously identified, most of which centrally operate by repressing the type 1 interferon response to increase viral infectivity of treated cells. In combination with oncolytic viruses, some of these compounds have been demonstrated to increase anti-tumour activity across various in vivo mouse models of cancer. These compounds have also been successfully applied commercially to improve the production of viral-based products. While different viral sensitizing compounds can be effective in a broad range of applications; their efficacy can fluctuate depending on the context owing to differences in their physicochemical properties as well as their compound-specific off-target effects. From a therapeutic standpoint in particular, the problem of low potency, side-effects, toxicity, and poor pharmacokinetics remain a primary obstacle to clinical translation.

As such, there is need to provide viral sensitizing compounds, for example, for the improvement of viral-based gene therapies and viral vector production.

### SUMMARY

It has been shown herein that neddylation-activating enzyme (NAE) inhibitors of the present application are effective to increase permissiveness of a cell to a virus, and thus exhibit viral sensitizing activity, with high potency and versatility.

Accordingly, the present application includes a method of increasing permissiveness of a cell to a virus, comprising administering an effective amount of a neddylation-activating enzyme (NAE) inhibitor, or a salt, solvate and/or prodrug thereof, to the cell.

The present application also includes a method of increasing permissiveness of a cell to genetic material encoding components of a virus, comprising administering an effective amount of a neddylation-activating enzyme (NAE) inhibitor, or a salt, solvate and/or prodrug thereof, to the cell in combination with provision of the genetic material encoding components of a virus to the cell.

The present application also includes a method of treating a disease, disorder or condition by increasing permissiveness of a cell to a virus comprising administering a therapeutically effective amount of a neddylation-activating enzyme (NAE) inhibitor, or a salt, solvate and/or prodrug thereof, and the virus or genetic material encoding the virus to a subject in need thereof.

The present application further includes a method of increasing the oncolytic activity of a virus comprising administering a therapeutically effective amount of a neddylation-activating enzyme (NAE) inhibitor, or a salt, solvate and/or prodrug thereof, with an oncolytic virus to a subject or cell in need thereof. The present application further includes a method of increasing the oncolytic activity of a virus comprising administering a therapeutically effective amount of a neddylation-activating enzyme (NAE) inhibitor, or a salt, solvate and/or prodrug thereof, and an oncolytic virus to a subject or cell in need thereof.

Further included is a method of treating a disease, disorder or condition by gene therapy comprising administering a therapeutically effective amount of a neddylation-activating enzyme (NAE) inhibitor, or a salt, solvate and/or prodrug thereof, and a gene therapy vector to a subject or cell in need thereof.

The present application also includes a method of increasing production of a virus by a cell comprising administering a neddylation-activating enzyme (NAE) inhibitor, or a salt, solvate and/or prodrug thereof, to the cell.

Also included is a method of increasing transduction of a virus into a cell comprising administering a neddylation-activating enzyme (NAE) inhibitor, or a salt, solvate and/or prodrug thereof, and the virus to the cell.

The present application also includes a method of increasing virally-encoded transgene expression comprising administering a neddylation-activating enzyme (NAE) inhibitor, or a salt, solvate and/or prodrug thereof, and the virus to a cell.

The present application further includes a method of increasing virus growth and/or virus spread in cells comprising administering a neddylation-activating enzyme (NAE) inhibitor, or a salt, solvate and/or prodrug thereof, to the cells in combination with provision of the virus to the cells.

Also included is a composition comprising a neddylation-activating enzyme (NAE) inhibitor, or a salt, solvate and/or prodrug thereof, and a virus or genetic material encoding components of a virus.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF DRAWINGS

The embodiments of the application will now be described in greater detail with reference to the attached drawings in which:

FIG. 1A, FIG. 1B, FIG. 1C, FIG. 1D, FIG. 1E and FIG. 1F show viral sensitizing activity results for pevonedistat, according to exemplary embodiments of the application, where: FIG. 1A shows representative phase and fluorescent images of human 786-0 cells pre-treated for 4 hours with pevonedistat (1 μM), then infected with VSVΔ51 expressing green fluorescent protein (GFP) for 24 h (scale bar=1000 μm) at Multiplicity of Infection (MOI) 0.01; FIG. 1B shows GFP expression by flow cytometry from cells treated as above and collected 24 hpi, FSC-H=forward scatter height;

FIG. 1C shows high-throughput titration results from supernatants of human 786-0 cells pre-treated for 4 hours with pevonedistat (30 nM-400 μM), then infected with VSVΔ51 expressing firefly luciferase (FLuc) at MOI 0.01 for 24 hours (n=2, *P<0.05 by two-tailed t-test to mock treated, infected cells); FIG. 1D shows expression of VSV-M and VSV-N genes at 24 hours post infection (hpi) following RNA extraction and qPCR of cells treated as above (n=3, mean±SD; ****P<0.0001 by Student's t-test); FIG. 1E shows relative titer (VEU/ml) from multi-step and single-step growth curves of 786-0 cells treated with pevonedistat, then infected with VSVΔ51-FLuc (MOI 0.001, 0.01, 3), supernatants quantified by high-throughput titration at specified timepoints (n=3, mean±SD; ***P<0.001, ****P<0.0001 by one-way ANOVA compares to the mock treated, mock infected condition); FIG. 1F shows 786-0 treated as above with pevonedistat (1 μM), then infected with VSVΔ51 (MOI 0.0001) for 1 hour following which an agarose overlay was added, and 48 hpi, cells were then stained with Coomassie blue and plaque diameters were measured at random (n=10, mean±SD; ****P<0.0001 by two-tailed t-test).

FIG. 2A, FIG. 2B, FIG. 2C, FIG. 2D, FIG. 2E and FIG. 2F show pevonedistat sensitization of various human and murine tumor types to VSVΔ51 according to exemplary embodiments of the application, where: FIG. 2A shows viral titers determined by plaque assay from various human and murine cell lines pre-treated with ±pevonedistat (1 μM), infected with VSVΔ51-GFP (MOI 0.01), and supernatants collected 24 hours post infection (hpi) (n=3, mean±SD; ****P<0.0001 by two-tailed t-test); FIG. 2B shows viral titers obtained from patient-derived ovarian (OVAPT) and glioma (PriGO) cells pre-treated with ±pevonedistat (1 μM), infected with VSVΔ51-GFP (MOI 0.01), and supernatants collected 48 hpi (n=3, mean±SD; ***P<0.001, ****P<0.0001 by two-tailed t-test); FIG. 2C shows viral titers obtained from primary murine hepatocytes isolated, cultured, treated with ±pevonedistat (1 μM) for 4 hours, infected with VSVΔ51-Fluc (MOI 0.05), and supernatants collected 40 hpi (n=3, mean±SD); FIG. 2D shows representative fluorescent images from CT26 (murine colon carcinoma) tumors grown subcutaneously in Balb/c mice for 24 days and subsequently excised, along with normal brain, lung, spleen and muscle tissues, then cored by punch biopsy to 2 mm×2 mm pieces which were treated with or without pevonedistat (10 μM) for 4 hours, then infected with VSVΔ51-GFP (3×10⁴ pfu/core) for 24 h (scale bar=1000 μm); FIG. 2E shows viral titers from the mouse samples in FIG. 2D where supernatants were taken 48 hpi and quantified for viral titer (n>6, mean±SD; ns=no significance, *P<****P<0.0001 by two-tailed t-test); FIG. 2F shows viral titers and representative fluorescent images obtained from various clinical human tumour samples which were cored as above and treated with pevonedistat (10 μM) for 4 hours, then infected with VSVΔ51-GFP (3×10⁴ pfu/core). At 24 hpi, representative fluorescent images were obtained (scale bar=1000 μm). Supernatants were taken 48 hpi and quantified for viral titer (n>6, mean±SD; ns=no significance, *P<0.05, ****P<0.0001 by two-tailed t-test).

FIG. 3A, FIG. 3B, FIG. 3C, FIG. 3D, FIG. 3E, FIG. 3F and FIG. 3G, show results for pevonedistat with VSVΔ51 inducing apoptosis via TNF-α pathways, amongst others, according to exemplary embodiments of the application, where: FIG. 3A shows a graph of relative metabolic activity as a function of concentration in human renal 786-0 carcinoma cells pre-treated with pevonedistat (30 nM-175 μM), infected with VSVΔ51 (MOI 0.01), and cells assessed at 48 hpi for cell viability using resazurin metabolic dye normalized to untreated, uninfected cells, and lethal dose (LD50) calculated (n=3, mean±SD; ****P<0.0001 by two-tailed t-test). FIG. 3B shows relative metabolic activity in various human and murine cell lines pre-treated±pevonedistat (1 μM), then infected±VSVΔ51 (MOI 0.01), and cell viability measured at 48 hpi (n=3, mean±SD; **P<0.01, ****P<0.0001 by one-way ANOVA); FIG. 3C, FIG. 3D, FIG. 3E shows results from 786-0 cells pre-treated with pevonedistat (1 μM) for 4 hours and infected with VSVΔ51 (MOI 0.01), in which FIG. 3C shows a western blot of cells lysed at 48 hpi and probed for cleaved caspase-3, caspase-3, cleaved PARP, PARP, and β-actin; FIG. 3D shows scatter plots of cells treated as above and collected 48 hpi and stained for Annexin V by flow cytometry, FSC-A=forward scatter area (n=3, mean±SD; ****P<0.0001 by one-way ANOVA); FIG. 3E shows cell viability of 786-0 cells treated±pevonedistat (1 μM)±Z-VAD-FMK (10 μM) for four hours, then infected±VSVΔ51 (MOI 0.01) and cell viability measured 48 hpi (n=3, mean±SD; ****P<0.0001 by two-way ANOVA); FIG. 3F shows cell viability of 786-0 cells pre-treated with pevonedistat (1 μM), then treated with either IFN-β (250 U/mL), TNF-α (10 ng/mL) or TLR-agonist poly I:C (1 μg/mL), and cell viability measured at 48 hpi (n=3, mean±SD; ns=no significance, ****P<0.0001 by two-way ANOVA); FIG. 3G shows luminescence at indicated hpi of 786-0 cells treated as above and measured for caspase-8 activity using a luciferase-based assay (n=3, mean±SD; ****P<0.0001 by one-way ANOVA).

FIG. 4A, FIG. 4B, FIG. 4C, FIG. 4D, FIG. 4E and FIG. 4F show results of administering pevonedistat to improve VSVΔ51 therapeutic efficacy in murine in vivo tumor models, for colon CT26WT and mammary 4T1 tumors implanted into the right flank of BALB/c mice according to exemplary embodiments of the application. Tumors were injected intratumorally with either dimethyl sulfoxide (DMSO) or pevonedistat (90 mg/kg) upon reaching sufficient size, and then injected intratumorally with VSVΔ51 (1×10⁸ pfu/tumor) after 4 hours. FIG. 4A and FIG. 4B show graphs of tumor volumes as a function of time, monitored every 2-3 days (n>10, mean±SEM; *P<0.05, **P<0.01 by one-way ANOVA), for colon CT26WT and mammary 4T1 tumors respectively; FIG. 4C and FIG. 4D show graphs of survival as a function of time in mice culled when tumor volumes reached 1500 mm³ for survival analysis, and Kaplan-Meier curves plotted and compared using the log-rank (Mantel-Cox) test (n=10 to 15), for colon CT26WT and mammary 4T1 tumors respectively; FIG. 4E and FIG. 4F show results from naïve mice and mice in remission from implanted CT26WT following administration of pevonedistat and VSVΔ51 that were then re-challenged with CT26WT cells in the opposite flank, where FIG. 4E shows a graph tumor volumes as a function of time, monitored every 2-3 days (n=4, mean±SEM; *P<0.05 by two-tailed t-test). FIG. 4F shows Kaplan-Meier curves plotted and compared using the log-rank test (n=4; *P<0.05).

FIG. 5A, FIG. 5B, FIG. 5C and FIG. 5D show that pevonedistat can impair the IFN-1 response in exemplary embodiments of the application, where two biological replicates of 786-0 cells pre-treated with ±pevonedistat (1 μM) for 4 hours, then infected±VSVΔ51 (MOI 0.01) for 24 hours were subject to RNA extraction and sequencing, followed by processing by KALLISTO pseudo-alignment and SLEUTH with data presented as normalized log 2-fold change in differential gene expression with P-values between the VSVΔ51 infected condition vs. the pevonedistat+VSVΔ51 condition; FIG. 5A shows a volcano plot for each gene, with notable hits named; FIG. 5B shows gene ontology (GO) terms where significantly downregulated (>2 log 2-fold change) gene expressions were processed by Gorilla; FIG. 5C shows a heat map of differential gene expressions related to the “Defense response to virus” GO term normalized to the mock treated, uninfected control condition; FIG. 5A shows a graph depicting Log 2-fold changes of genes as a function of the “Response to type 1 interferon” GO term, in which overall gene expressions levels were compared between conditions (n=2; ****P<0.0001 by one-way ANOVA).

FIG. 6A, FIG. 6B, FIG. 6C, FIG. 6D, FIG. 6E, FIG. 6F, FIG. 6G and FIG. 6H, show that the viral sensitizing activity of pevonedistat occurs through inhibition of STAT1 expression according to exemplary embodiments of the application, where: FIG. 6A shows a graph of E-value and percentage of regulated genes, where differential gene expressions from performed RNA-sequencing between the mock vs. pevonedistat condition (uninfected) and between the VSVΔ51 infected condition vs. the pevonedistat+VSVΔ51 condition (infected) were input into TFactS to predict involved transcription factors; FIG. 6B shows a heat map of log 2-fold change of genes regulated by the STAT1 transcription factor in cells treated as above; FIG. 6C and FIG. 6D show results on human renal 786-0 carcinoma cells pre-treated with ±pevonedistat (1 μM) for 4 hours, then infected±VSVΔ51 (MOI 0.1), where FIG. 6C shows the western blot of cells lysed at 24 hpi and probed for proteins as indicated and FIG. 6D shows relative mRNA expression as a function of time for cells lysed at 8, 16, and 24 hpi and expression of STAT1 and IRF7 quantified by qPCR (n=3, mean±SD; ****P<0.0001 by one-way ANOVA between the VSVΔ51 infected condition vs. pevonedistat+VSVΔ51 condition); FIG. 6E, FIG. 6F, FIG. 6G and FIG. 6H show results for 786-0 cells transfected either with scramble, control siRNA or siRNA targeting NEDD8, transfected cells then pre-treated±with pevonedistat (1 μM) for 4 hours, and infected with ±VSVΔ51 (MOI 0.05), where FIG. 6E shows western blot of cells lysed at 24 hpi and probed for NEDD8 protein expression; FIG. 6F shows viral titers from supernatants of cells transfected with either scramble siRNA or siRNA targeting NEDD8 and pre-treated±pevonedistat (1 μM) for 4 hours, then infected±VSVΔ51 taken 24 hpi and quantified for viral titer by plaque assay (n=3, mean±SD; ns=no significance, ****P<0.0001 by two-way ANOVA); FIG. 6G shows representative fluorescent images of the latter taken 24 hpi (scale bar=1000 μm); FIG. 6H shows relative mRNA expression for lysates taken at 24 hpi and probed for STAT1 and IRF7 expression by qPCR (n=3, mean±SD; *P<0.05, ****P<0.0001 by two-way ANOVA).

FIG. 7A, FIG. 7B, FIG. 7C, FIG. 7D, FIG. 7E, FIG. 7F, FIG. 7G, FIG. 7H and FIG. 7I show that pevonedistat can inhibit NF-κB to suppress IFN-β in a neddylation-independent manner according to exemplary embodiments of the application, where: FIG. 7A shows a western blot from human renal 786-0 carcinoma cells pre-treated for 4 hours with pevonedistat (1 μM), then infected with VSVΔ51 (MOI 0.01), and cells lysed after 24 hpi and fractionated to separate nuclear and cytoplasmic proteins, and fractions probed for indicated proteins; FIG. 7B and FIG. 7C show results for 786-0 cells seeded on glass coverslips, and pre-treated for 4 hours with pevonedistat (10 μM), where FIG. 7B shows florescent images for cells then treated with human TNF-α (10 ng/mL) for 30 minutes or FIG. 7C shows cells then treated with VSVΔ51 (MOI 1) for 6 hours, cells fixed and immunostained for NF-κB and nuclei (DAPI), nuclear NF-κB intensity quantified (n=3, mean±SD; ****P<0.0001); FIG. 7D and FIG. 7E show results for 786-0 cells pre-treated with ±pevonedistat (1 μM) for 4 hours, then infected with ±VSVΔ51 (MOI 0.01), where FIG. 7D shows a heat map of relative mRNA expression from RNA extracted from cells 24 hpi and probed for indicated genes by qPCR, and FIG. 7E shows IFN-β secretion from supernatants quantified at 24 hpi by ELISA (n=3, mean±SD; ****P<0.0001 by two-way ANOVA); FIG. 7F shows NF-κB nuclear intensity from siNEDD8 or scramble siRNA transfected cells pre-treated with ±pevonedistat (10 μM), then infected with ±VSVΔ51-GFP (MOI 1) for 6 hours, fixed and immunostained for NF-κB and nuclei (DAPI), images were processed and quantified for mean nuclear NF-κB signal (n=3, mean±SD; ns=no significance, ****P<0.0001 by one-way ANOVA); FIG. 7G shows relative mRNA expression from cells pre-treated with pevonedistat (1 μM) for 4 hours, then infected with VSVΔ51 (MOI 0.01), RNA extracted at 24 hpi and probed for IFN-β gene expression by qPCR (n=3, mean±SD; ns=no significance, ***P<0.001 by one-way ANOVA); FIG. 7H shows relative mRNA expression from 786-0 cells pre-treated for 4 hours with pevonedistat (1 μM), then treated with human IFN-β (1000 U/mL) for 6 hours, RNA extracted and probed for STAT1 gene expression (n=3, mean±SD; *P<0.05 by one-way ANOVA).

FIG. 8 shows a proposed graphical model exemplary of pevonedistat's mechanism of action.

FIG. 9A and FIG. 9B show results of viral sensitizing ability with VSVΔ51 for covalent and non-covalent NAE inhibitors according to exemplary embodiments of the application, in human 786-0 renal carcinoma cells pre-treated with a concentration range of TAS4464 for 4 hours, then infected with VSVΔ51 expressing firefly luciferase (VSVΔ51-FLuc), MOI 0.05, where FIG. 9A shows a graph of viral titer as a function of concentration of TAS4464 in cells analyzed for viral titer by high-throughput titration at 48 hpi, with dotted line as baseline infection without drug (n=3, *P<0.05, ****P<0.0001 by two-way ANOVA); FIG. 9B shows a graph of relative metabolic activity as a function of concentration of TAS4464 where cell viability was assessed using Alamar blue assay expressed relative to the uninfected, untreated condition (n=3, *P<0.05, **P<0.01 by two-way ANOVA).

FIG. 10A and FIG. 10B show results of viral sensitizing ability with VSVΔ51 for covalent and non-covalent NAE inhibitors according to exemplary embodiments of the application, in human 786-0 renal carcinoma cells pre-treated with a concentration range of ZM223 for 4 hours, then infected with VSVΔ51 expressing firefly luciferase (VSVΔ51-FLuc), MOI 0.05, where FIG. 10A shows a graph of viral titer as a function of concentration of ZM223 in cells analyzed at 48 hpi for viral titer by high-throughput titration, with dotted line as baseline infection without drug (n=3, *P<0.05, ****P<0.0001 by two-way ANOVA); FIG. 10B shows a graph of relative metabolic activity as a function of concentration of ZM223 where cell viability was assessed using Alamar blue assay expressed relative to the uninfected, untreated condition (n=3, *P<0.05, **P<0.01 by two-way ANOVA).

FIG. 11A, FIG. 11B and FIG. 11C show results of viral sensitizing ability with VSVΔ51 via IFN-1 repression for covalent and non-covalent NAE inhibitors according to exemplary embodiments of the application, in 786-0 renal carcinoma cells pre-treated with the indicated dose of pevonedistat, TAS4464 or ZM223 for 4 hours, then infected with VSVΔ51-GFP (MOI 0.01) for 24 hours, where FIG. 11A shows representative fluorescent images; FIG. 11B shows viral titer from supernatants analyzed by plaque assay (n=3, ****P<0.0001 by one-way ANOVA); FIG. 11C shows IFN-β secretion from supernatants measured by ELISA (n=3, ****P<0.0001 by one-way ANOVA).

FIG. 12A, FIG. 12B, FIG. 12C and FIG. 12D show results of wild-type VSV and MG-1 infectivity from Vero cells treated with a range of concentrations of pevonedistat as indicated for 4 hours, then infected with either VSV-wild type-FLuc (MOI 0.001) or MG-1 (MOI 0.01) according to exemplary embodiments of the application, where FIG. 12A shows a graph of viral titer as a function of concentration assessed by high-throughput titration; FIG. 12B shows a graph of fold change in viral titer, assessed by high-throughput titration, as a function of concentration; FIG. 12C shows viral titer at various concentrations assessed by plaque assay; FIG. 12D shows a graph of fold change in viral titer, assessed by plaque assay, as a function of concentration.

FIG. 13A, FIG. 13B and FIG. 13C show results of viral sensitizing ability of cancer cells to other viral vectors according to exemplary embodiments of the application, where FIG. 13A shows fluorescent images from 786-0 cells pre-treated with pevonedistat (1 μM) for 4 hours, then infected with measles (MeV) tagged with GFP (MOI 0.3), and fluorescent images taken at 48 hpi; FIG. 13B shows viral titer from 786-0 cells pre-treated with pevonedistat (100 nM or 1 μM) for 4 hours, then infected with Sindbis (MOI 0.01), and supernatants analyzed for viral titer by plaque assay at 48 hpi (n=3, *P<0.05, ***P<0.001 by one-way ANOVA); FIG. 13C shows luminescence from human lung A549 adenocarinoma cells pre-treated with pevonedistat (10 nM, 100 nM, 1 μM) for 4 hours, then infected with replication-defective adenovirus serotype 5 (Ad5) tagged with FLuc at MOI 10, and, supernatants assessed for luminescence activity at 48 hpi, indicative of Ad5 quantity (n=3, **P<0.01, ***P<0.001 by one-way ANOVA).

FIG. 14A, FIG. 14B and FIG. 14C show viral sensitizing activity of pevonedistat in Human HT29 colorectal adenocarcinoma cells. Human HT29 colorectal adenocarcinoma cells were pre-treated with pevonedistat (1 μM) for 4 hours, then infected with VSVΔ51 (MOI 0.01). FIG. 14A shows cells lysed 24 hpi and probed for indicated proteins by western blot. FIG. 14B shows RNA was extracted from cells 24 hpi; indicated genes were quantified by qPCR (n=3,****P<0.0001 by two-way ANOVA). FIG. 14C shows human HT29 colorectal adenocarcinoma cells pre-treated with pevonedistat (1 μM) for 4 hours, then infected with VSVΔ51 (MOI 0.01). At 8, 16 and 24 hpi, cells were lysed and RNA was extracted. Indicated genes were quantified by qPCR (n=3, ****P<0.0001 by two-way ANOVA).

FIG. 15 shows viral sensitizing activity of pevonedistat ex vivo xenograft experiments. Human primary ovarian AF2068 cells, breast JIMT1 or ovarian SKOV3 cells were implanted into immunodeficient nude mice to create a xenograft model. Tumors were extracted after tumors reached 1000 mm³, cored and treated ex vivo with pevonedistat (10 μM) for 4 hours, then infected with VSVΔ51-GFP (3×10⁴ pfu/core). At 24 hpi, representative fluorescent images were obtained (scale bar=1000 μm).

FIG. 16A and FIG. 16B show a validation of siNEDD8 findings with siUBA3. 786-0 cells were transfected either with scramble, control siRNA or siRNA targeting UBA3 or NEDD8. Transfected cells were then pre-treated±pevonedistat (1 μM) for 4 hours, then infected±VSVΔ51 (MOI 0.05). In FIG. 16A cells were lysed at 24 hpi and probed for UBA3 protein expression by western blot. In FIG. 16B supernatant was taken 24 hpi and quantified for viral titer by plaque assay (n=3, mean±SD; ns=no significance, ****P<0.0001 by two-way ANOVA).

FIG. 17A, FIG. 17B and FIG. 17C also show a validation of siNEDD8 findings with siUBA3. In FIG. 17A, 786-0 cells were treated with pevonedistat (1 μM) for 4 hours or transfected with siRNA against UBA3 (20 nM) for 2 days. Cells were then infected with VSVΔ51 for 24 hours, then lysed and probed for IκB-α by western blot. Densitometry was performed to quantify band intensity and normalized to the loading control. In FIG. 17B and FIG. 17C 786-0 cells were seeded on glass coverslips and transfected with siRNA against UBA3 for two days. FIG. 17B shows ells were then treated with TNF-α (30 ng/mL) for 30 minutes, then fixed and stained for NF-κB by immunocytochemistry. Representative immunofluorescent images are shown. FIG. 17C shows NF-κB nuclear intensity quantified and plotted (n=3, ****P<0.0001 by two-way ANOVA).

FIG. 18A and FIG. 18B show viral sensitizing activity of pevonedistat in vivo in melanoma B16 tumors and ovarian ID8-Tp53−/− (F3) cells. FIG. 18A shows melanoma B16 tumors overexpressing OVA antigen (B16-OVA) implanted into the right flank of C57BL/6 mice. When tumor volume reached 100 mm³, mice were injected intratumorally with three doses of pevonedistat (90 mg/kg), then VSVΔ51 (1×10⁸ pfu/tumor), spaced one day apart. Mice were culled at 1500 mm³ and Kaplan-Meier survival curves were plotted (n=10). Lines from left to right show DMSO/PBS, Pevonedistat/PBS, DMS/VSVΔ51 and Pevonedistat/VSVΔ51. FIG. 18B shows ovarian ID8-Tp53−/− (F3) cells injected intraperitoneally and allowed to achieve sufficient tumor burden. Mice were then injected intraperitoneally with pevonedistat (90 mg/kg), then VSVΔ51 (1×10⁸ pfu) three times, spaced one day apart. Tumor burden was assessed by luminescence signal measurements taken with the in vivo imaging system (IVIS) and quantified at day 21 and 28 after first treatment (n=4-5, mean±SEM, *P<0.05, **P<0.01 by two-way ANOVA, all other comparisons not significant).

FIG. 19 shows the effect of pevonedistat on AAV2-Fluc production as assessed by a functional titer assay.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. The term “and/or” with respect to salts. solvates and/or prodrugs thereof means that the compounds of the application exist as individual salts, solvates and prodrugs, as well as a combination of, for example, a salt of a solvate of a compound of the application.

The term “compound of the application” or “compound of the present application” and the like as used herein refers to any neddylation-activating enzyme (NAE) inhibitor, including those disclosed herein as well as salts, solvates and/or prodrugs thereof.

The term “composition of the application” or “composition of the present application” and the like as used herein refers to a composition comprising one or more compounds of the application and optionally one or more viruses.

The term “suitable” as used herein means that the selection of the particular composition or conditions would depend on the specific steps to be performed, the identity of the components to be transformed and/or the specific use for the compositions, but the selection would be well within the skill of a person trained in the art.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, means a saturated carbocyclic group containing from 3 to 10 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains either 6 to 10 carbon atoms.

The term “heterocyclyl” as used herein, whether it is used alone or as part of another group, refers to cyclic groups containing at least one non-aromatic ring containing from 3 to 10 atoms in which one or more of the atoms are a heteroatom selected from O, S and N and the remaining atoms are C. Heterocyclyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocyclyl groups are optionally benzofused.

The term “heteroaryl” as used herein, whether it is used alone or as part of another group, refers to cyclic groups containing at least one heteroaromatic ring containing 5-10 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocyclyl and cycloalkyl groups, contain one or more than one ring (i.e. are polycyclic). When a cyclic group contains more than one ring, the rings may be fused, bridged, spirofused or linked by a bond.

The term “fluoroalkyl” refers to the substitution of one or more, including all, available hydrogens in an alkyl group with fluoro.

The terms “halo” or “halogen” as used herein, whether it is used alone or as part of another group, refers to a halogen atom and includes fluoro, chloro, bromo and iodo.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition.

The term “administered” as used herein means administration of a therapeutically effective amount of a compound, or one or more compounds, or a composition of the application to a cell either in cell culture or in a subject.

As used herein, the term “effective amount” or “therapeutically effective amount” means an amount of a compound, or one or more compounds, of the application that is effective, at dosages and for periods of time necessary to achieve a desired result.

The term “cancer” as used herein refers to cellular-proliferative disease states.

The terms “sensitization” or “sensitizing” as used herein, in the context of a cell, refers to a decreased or altered cellular response to an outside agent such that the outside agent has an increased or altered effect on the cell. Where the outside agent is a virus, the terms “sensitization” or “sensitizing” (also referred to as “viral sensitization” or “viral sensitizing”) refers to a decreased or altered cellular response to the virus, thereby increasing the ability of the virus to infect and/or replicate in the cell or be produced by a cell.

The term “permissiveness” as used herein, in the context of a cell, refers to an increase in the uptake of an outside agent by the cell and/or an increase in the activity of the outside agent in the cell and/or a reduction in cellular defenses that would otherwise inhibit the expression, replication or stability of the outside agent in the cell. When the outside agent is a virus, “permissiveness” refers to the ability of a virus to infect and/or transduce a cell.

The term “genetic material encoding components of a virus” refers to a nucleic acid, and chemically modified variants thereof, that carry viral-like sequences of nucleotides. The sequences encode viral-like proteins and/or functional sequences for targeting, integration, promotion, etc.

The term “increase” or “increasing” as used herein refers to any detectable increase or enhancement in a function or characteristic in the presence of one or more test variables, compared to otherwise the same conditions except in the absence of the one or more test variables.

The term “decrease” or “decreasing” as used herein refers to any detectable decrease or reduction in a function or characteristic in the presence of one or more test variables, compared to otherwise the same conditions except in the absence of the one or more test variables.

### II. Compositions of the Application

It has been shown herein that neddylation-activating enzyme (NAE) inhibitors of the present application (compounds of the application) are effective to increase permissiveness of a cell to a virus, and thus exhibit viral sensitizing activity, with high potency and versatility.

Accordingly, the present application includes a composition comprising a neddylation-activating enzyme (NAE) inhibitor, or a pharmaceutically acceptable salt, solvate and/or prodrug thereof (i.e. a compound of the application) and a virus or genetic material encoding components of the virus, optionally with a pharmaceutically acceptable carrier or excipient.

In some embodiments, the NAE inhibitor (or compound of the application) is a compound of formula (I), or a salt, solvate and/or prodrug thereof:

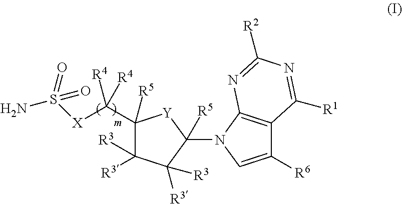

or a salt, solvate and/or prodrug thereof, or a combination thereof, wherein:
X is CH₂, CHF, CF₂, NH or O;
Y is O, S or CH₂;
R¹ is H, Cl, Br, F, I, NR⁷R⁸, R⁹, SH, SCH₃, SR¹⁰, OH, OCH₃ or OR¹⁰;
R² is H, Cl, Br, F, I, N(R⁸), CN, OR⁸, SR⁸, or an optionally substituted C₁₋₄alkyl;
each R³ is independently H, F, C₁₋₄alkyl or C₁₋₄fluoroalkyl;
each R^{3′} is independently H, CN, N₃, OH, OR¹¹, NH₂, NHR¹¹, NHCO₂R¹¹, NHC(O)R¹¹, C(O)NHR¹¹, OC(O)NHR¹¹, OC(O)R¹¹, OC(O)OR¹¹, C₁₋₄fluoroalkyl, or C₁₋₄alkyl optionally substituted with one or two substituents each being independently OR¹², NR¹³R¹⁴, CO₂R¹² or C(O)NR¹³R¹⁴
each R⁴ is independently H, F, C₁₋₄alkyl or C₁₋₄fluoroalkyl; or two R⁴ taken together with the carbon to which they are attached form a 3- to 6-membered ring; or two R⁴ form ═O;
each R⁵ is independently H or C₁₋₄alkyl;
or one R⁴ taken with R⁵ on the adjacent carbon form with the intervening carbon atoms a 3- to 6-membered ring;
R⁶ is H, halo or an optionally substituted C₁₋₄alkyl;
R⁷ is an optionally substituted C₆₋₁₀aryl, C₅₋₁₀heteroaryl or C₃₋₁₀heterocyclyl;
R⁸ is H or C₁₋₄alkyl;
R⁹ is VZR¹⁵, VZR¹⁶, R¹⁷, or an optionally substituted C₁₋₁₀alkyl, C₆₋₁₀aryl, C₅₋₁₀heteroaryl or C₃₋₁₀heterocyclyl, wherein the heteroaryl is attached at a carbon atom;
R¹⁰ is an unsubstituted C₂₋₁₀alkyl, a substituted C₁₋₁₀alkyl, or optionally substituted C₆₋₁₀aryl, C₅₋₁₀heteroaryl or C₃₋₁₀heterocyclyl;
R¹¹ is an optionally substituted C₁₋₁₀alkyl, C₆₋₁₀aryl, C₅₋₁₀heteroaryl or C₃₋₁₀heterocyclyl;
R¹² is H, C₁₋₄alkyl, C₁₋₄fluoroalkyl, or an optionally substituted C₆₋₁₀aryl or C₁₋₄alkyleneC₆₋₁₀aryl;
R¹³ is H, C₁₋₄alkyl, C₁₋₄fluoroalkyl, or an optionally substituted C₁₋₄alkyleneC₆₋₁₀aryl;
R¹⁴ is H, C₁₋₄alkyl, C₁₋₄fluoroalkyl, an optionally substituted C₁₋₄alkyleneC₆₋₁₀aryl or an optionally substituted 5- or 6-membered aryl, heteroaryl or heterocyclyl;
R¹⁵ is an optionally substituted C₆₋₁₀aryl, C₅₋₁₀heteroaryl, C₃₋₁₀heterocyclyl or C₃₋₁₀cycloalkyl;
R¹⁶ is halo, NO₂, CN, OR¹⁸, SR¹⁹, N(R²⁰)₂, N(R²⁰)C(O)R¹⁹, N(R²⁰)C(O)NR²⁰, N(R²⁰)CO₂R¹⁸, OCO₂R¹⁸, OC(O)N(R²⁰)₂, OC(O)R¹⁸, N(R²⁰)N(R²⁰)₂, N(R²⁰)OR¹⁹, N(R²⁰)SO₂R¹⁹, N(R²⁰)SO₂N(R²⁰)₂, CR¹⁸═C(R¹⁸)₂, —C≡CR¹⁸, S(O)R¹⁹, SO₂R¹⁹, SO₂N(R²⁰)₂, CR¹⁸═NOR¹⁸, CO₂R¹⁸, C(O)C(O)R¹⁸, C(O)R¹⁸, C(O)N(R²⁰)₂, C(═NR²⁰)N(R²⁰)₂ or C(═NR²⁰)OR¹⁸,
R¹⁷ is NO₂, CN, SO₂R¹⁹, SO₂N(R²⁰)₂, SO₂, N(R²⁰)₂, C(R¹⁸)═NOR¹⁸, N(R²⁰)C(O)R¹⁹, N(R²⁰)C(O)N(R²⁰)₂, OCO₂R¹⁸, OC(O)N(R²⁰)₂, OC(O)R¹⁸, CO₂R¹⁸, C(O)C(O)R¹⁸, C(O)R¹⁸, C(O)N(R²⁰)₂, C(═NR²⁰)N(R²⁰)₂, C(═NR²⁰)OR¹⁸, N(R²⁰)N(R²⁰)₂, N(R²⁰)OR¹⁹, N(R²⁰)SO₂R¹⁹ or N(R²⁰)SO₂N(R²⁰)₂;
V is —SO₂—, —SO—, —CO₂—, —C(O)—, —C(NR¹⁸)═N—, —C(═NR¹⁸)N(R¹⁸)—, —C(OR¹⁰)═N—, —C(O)N(R¹⁸)—, —N(R¹⁸)C(O)—, —N(R¹⁸)C(O)N(R¹⁸)—, —N(R¹⁸)SO₂—, —N(R¹⁸)SO₂N(R¹⁸)—, —N(R¹⁸)CO₂—, —SO₂N(R¹⁸)—, —OC(O)—, —OCO₂—, —OC(O)N(R¹⁸)— or —N(R¹⁸)N(R¹⁸)—;
Z is an optionally substituted alkylene, optionally interrupted with —CR¹⁸CR¹⁸—, —C—O—, —S—, —C≡C—, —N(R¹⁸)—, —N(R¹⁸)C(O)—, —N(R¹⁸)CO₂—, —C(O)N(R¹⁸)—, —C(O)—, —C(O)—C(O)—, —CO₂—, —OC(O)—, —OCO₂—, —N(R¹⁸)C(O)N(R¹⁸)—, —N(R¹⁸)N(R¹⁸)—, —OC(O)N(R¹⁸)—, —SO₂—, —SO—, —N(R¹⁸)SO₂— or —SO₂N(R¹⁸)—;
each R¹⁸ is independently H or an optionally substituted C₁₋₁₀alkyl, C₆₋₁₀aryl, C₅₋₁₀heteroaryl or C₃₋₁₀heterocyclyl;
each R¹⁹ is independently an optionally substituted C₁₋₁₀alkyl or C₆₋₁₀aryl;
each R²⁰ is independently an optionally substituted C₁₋₁₀alkyl, C₆₋₁₀aryl, C₅₋₁₀heteroaryl or C₃₋₁₀heterocyclyl, or two R²⁰ on the same nitrogen atom are taken together with the nitrogen atom to form an optionally substituted 5- to 8-membered heterocyclyl ring having zero to two additional heteroatoms selected from N, O and S;
wherein each recitation of optionally substituted aryl refers to one or more substituents independently selected from halogen, —NO₂, —CN, —R²¹, —C(R²¹)═C(R²¹)₂, —C≡C—R²¹, —OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂;
wherein each recitation of optionally substituted heteroaryl refers to one or more substituents on an unsaturated carbon atom independently selected from halogen, —NO₂, —CN, —R²¹, —C(R²¹)═C(R²¹)₂, —C≡C—R²¹, —OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂; or on a substitutable nitrogen atom, from R²¹, —N(R²¹)₂, —C(O)R²¹, —CO₂R²¹, —C(O)—C(O)R²¹, —C(O)CH₂C(O)R²¹, —SO₂R²¹, —SO₂N(R²¹)₂, —C(═S)N(R²¹)₂, —C(═NH)N(R²¹)₂ and —NR²¹SO₂R²¹;
Wherein each recitation of optionally substituted alkyl refers to one or more substituents independently selected from halogen, —NO₂, —CN, —R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂, ═O, ═S, ═C(R²¹)₂, ═N—N(R²³)₂, ═N—OR²¹, ═N—NHC(O)R²¹, ═N—NHCO₂R²², ═N—NHSO₂R²² and =N—R²¹;
wherein each recitation of optionally substituted heterocyclyl refers to one or more substituents on an unsaturated carbon atom independently selected from halogen, —NO₂, —CN, —R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂, ═O, ═S, ═C(R²¹)₂, =N—N(R²³)₂, =N—OR²¹, ═N—NHC(O)R²¹, =N—NHCO₂R²², =N—NHSO₂R²² and =N—R²¹; or on a substitutable nitrogen atom, from R²¹, —N(R²¹)₂, —C(O)R²¹, —CO₂R²¹, —C(O)—C(O)R²¹, —C(O)CH₂C(O)R²¹, —SO₂R²¹, —SO₂N(R²¹)₂, —C(═S)N(R²¹)₂, —C(═NH)N(R²¹)₂ and —NR²¹SO₂R²¹;
wherein
each R²¹ is independently H, C₁₋₁₀alkyl, C₆₋₁₀aryl, C₅₋₁₀heteroaryl or C₃₋₁₀heterocyclyl;
each R²² is independently C₁₋₁₀alkyl or C₆₋₁₀aryl;
each R²³ is independently H, C₁₋₁₀alkyl, C₆₋₁₀aryl, C₅₋₁₀heteroaryl or C₃₋₁₀heterocyclyl, or two R²³ on the same nitrogen atom are taken together with the nitrogen atom to form a 5- to 8-membered ring having zero to two additional heteroatoms selected from N, O and S;
and m is 1, 2 or 3.

In some embodiments, the NAE inhibitor (or compound of the application) is selected from a compound in Table 1, or a salt, solvate and/or prodrug thereof.

TABLE 1
Neddylation-activating enzyme (NAE) inhibitors

| Compound | | |
|---|---|---|
| ID/name | IUPAC Name | Structure |
| Pevonedistat, MLN4924 Compound 1 | ((1S,2S,4S)-4- (4-(((S)-2,3- dihydro-1H-inden- 1-yl)amino)- 7H-pyrrolo[2,3- d]pyrimidin-7-yl)- 2-hydroxycyclopentyl) methyl sulfamate | 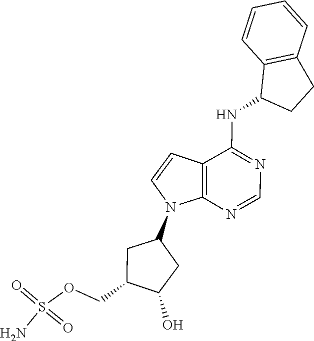 |
| TAS4464 Compound 2 | N/A | 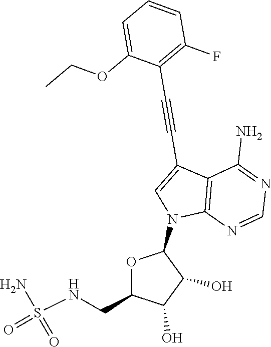 [SMILES: CCOc1cccc(F)c1C#Cc1cn([C@@H]2O[C@H](CNS(N)(=O)=O)[C@@H](O)[C@H]2O)c2ncnc(N)c12] |
| Compound 3 | ((2R,3S,4R,5R)- 5-(6-(((S)-2,3- dihydro-1H-inden- 1-yl)amino)- 9H-purin-9-yl)-3,4- dihydroxytetrahydrofuran-2- yl)methyl sulfamate | 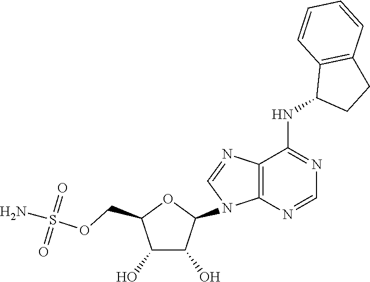 [SMILES: NS(=O)(=O)OC[C@H]1O[C@@H](n2cnc3c(N[C@H]4CCc5ccccc54)ncnc32)[C@H](O)[C@@H]1O] |
| Compound 4 | ((2R,3S,4R,5R)- 5-(6-amino-9H- purin-9-yl)-3,4- dihydroxytetrahydrofuran-2- yl)methyl sulfamate | 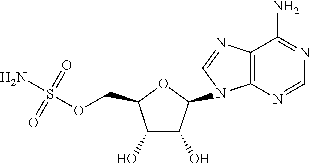 [SMILES: Nc1ncnc2c1ncn2[C@@H]1O[C@H](COS(N)(=O)=O)[C@@H](O)[C@H]1O] |
| Compound 5 | ((2R,3S,4R,5R)- 3,4-dihydroxy-5- (6-(methylamino)- 9H-purin-9- yl)tetrahydrofuran- 2-yl)methyl sulfamate | 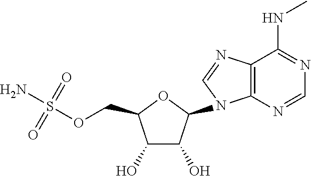 [SMILES: CNc1ncnc2c1ncn2[C@@H]1O[C@H](COS(N)(=O)=O)[C@@H](O)[C@H]1O] |
| Compound 6 | ((2R,3S,4R,5R)- 3,4-dihydroxy-5- (6-(propylamino)- 9H-purin-9- yl)tetrahydrofuran- 2-yl)methyl sulfamate | 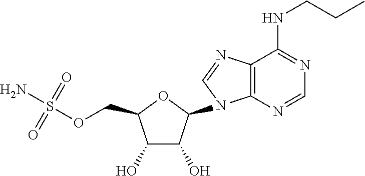 [SMILES: CCCNc1ncnc2c1ncn2[C@@H]1O[C@H](COS(N)(=O)=O)[C@@H](O)[C@H]1O] |
| Compound 7 | ((2R,3S,4R,5R)-5-(6- (allylamino)-9H- purin-9-yl)-3,4- dihydroxytetrahydrofuran- 2- yl)methyl sulfamate | 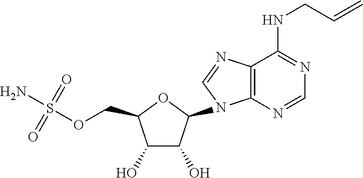 [SMILES: C=CCNc1ncnc2c1ncn2[C@@H]1O[C@H](COS(N)(=O)=O)[C@@H](O)[C@H]1O] |
| Compound 8 | ((2R,3S,4R,5R)-5-(6- (butylamino)-9H- purin-9-yl)-3,4- dihydroxytetrahydrofuran- 2- yl)methyl sulfamate | 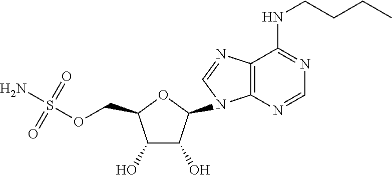 [SMILES: CCCCNc1ncnc2c1ncn2[C@@H]1O[C@H](COS(N)(=O)=O)[C@@H](O)[C@H]1O] |
| Compound 9 | ((2R,3S,4R,5R)- 3,4-dihydroxy-5- (6-(pentylamino)- 9H-purin-9- yl)tetrahydrofuran- 2-yl)methyl sulfamate | 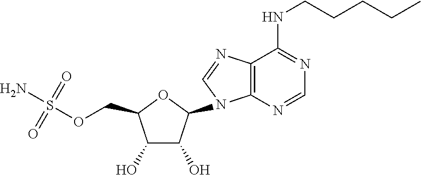 [SMILES: CCCCCNc1ncnc2c1ncn2[C@@H]1O[C@H](COS(N)(=O)=O)[C@@H](O)[C@H]1O] |
| Compound 10 | ((2R,3S,4R,5R)-5-(6- (hexylamino)-9H- purin-9-yl)-3,4- dihydroxytetrahydrofuran- 2- yl)methyl sulfamate | 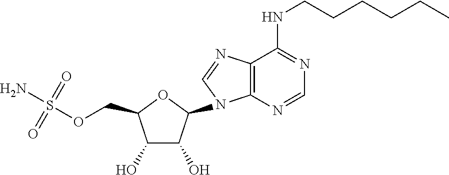 [SMILES: CCCCCCNc1ncnc2c1ncn2[C@@H]1O[C@H](COS(N)(=O)=O)[C@@H](O)[C@H]1O] |
| Compound 11 | ((2R,3S,4R,5R)-5-(6- (heptylamino)-9H- purin-9-yl)-3,4- dihydroxytetrahydrofuran- 2- yl)methyl sulfamate | 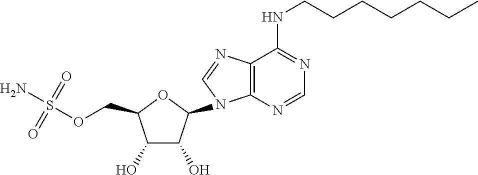 [SMILES: CCCCCCCNc1ncnc2c1ncn2[C@@H]1O[C@H](COS(N)(=O)=O)[C@@H](O)[C@H]1O] |
| Compound 12 | ((2R,3S,4R,5R)- 3,4-dihydroxy-5- (6-(isopropylamino)- 9H-purin-9- yl)tetrahydrofuran- 2-yl)methyl sulfamate | 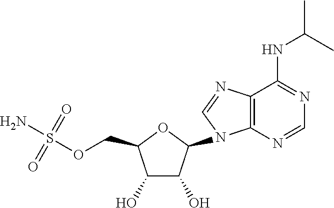 [SMILES: CC(C)Nc1ncnc2c1ncn2[C@@H]1O[C@H](COS(N)(=O)=O)[C@@H](O)[C@H]1O] |
| Compound 13 | ((2R,3S,4R,5R)- 3,4-dihydroxy-5- (6-(isobutylamino)- 9H-purin-9- yl)tetrahydrofuran- 2-yl)methyl sulfamate | 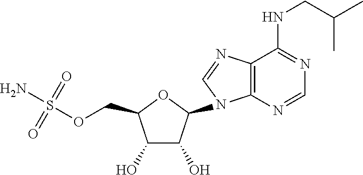 [SMILES: CC(C)CNc1ncnc2c1ncn2[C@@H]1O[C@H](COS(N)(=O)=O)[C@@H](O)[C@H]1O] |
| Compound 14 | ((2R,3S,4R,5R)- 3,4-dihydroxy-5- (6-(isopentylamino)- 9H-purin-9- yl)tetrahydrofuran- 2-yl)methyl sulfamate | 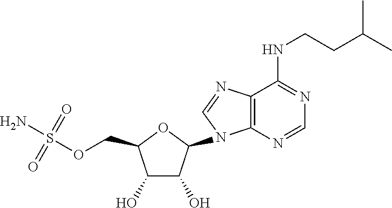 [SMILES: CC(C)CCNc1ncnc2c1ncn2[C@@H]1O[C@H](COS(N)(=O)=O)[C@@H](O)[C@H]1O] |
| Compound 15 | ((2R,3S,4R,5R)- 3,4-dihydroxy-5- (6-((4-methylpentan- 2-yl)amino)- 9H-purin-9-yl) tetrahydrofuran-2- yl)methyl sulfamate | 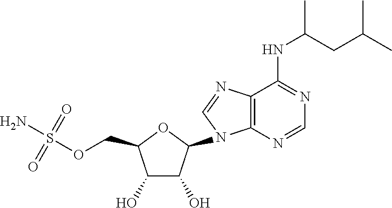 [SMILES: CC(C)CC(C)Nc1ncnc2c1ncn2[C@@H]1O[C@H](COS(N)(=O)=O)[C@@H](O)[C@H]1O] |
| Compound 16 | ((2R,3S,4R,5R)- 5-(6-((3,3- dimethylbutyl) amino)-9H-purin- 9-yl)-3,4- dihydroxytetrahydrofuran- 2- yl)methyl sulfamate | 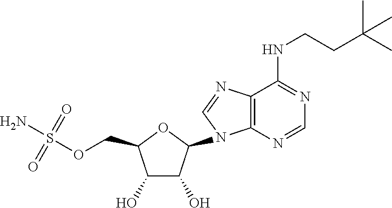 [SMILES: CC(C)(C)CCNc1ncnc2c1ncn2[C@@H]1O[C@H](COS(N)(=O)=O)[C@@H](O)[C@H]1O] |
| Compound 17 | ((2R,3S,4R,5R)- 3,4-dihydroxy-5- (6-((2,4,4- trimethylpentan-2- yl)amino)-9H-purin-9- yl)tetrahydrofuran- 2-yl)methyl sulfamate | 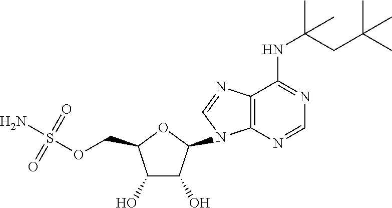 [SMILES: CC(C)(C)CC(C)(C)Nc1ncnc2c1ncn2[C@@H]1O[C@H](COS(N)(=O)=O)[C@@H](O)[C@H]1O] |
| Compound 18 | ((2R,3S,4R, 5R)-5-(6-(sec- butylamino)-9H- purin-9-yl)-3,4- dihydroxytetrahydrofuran- 2- yl)methyl sulfamate | 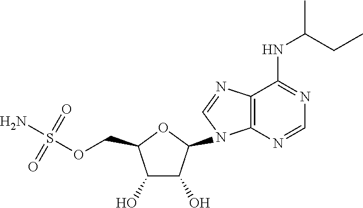 [SMILES: CCC(C)Nc1ncnc2c1ncn2[C@@H]1O[C@H](COS(N)(=O)=O)[C@@H](O)[C@H]1O] |
| Compound 19 | ((2R,3S,4R,5R)- 5-(6-(heptan-2- ylamino)-9H-purin- 9-yl)-3,4- dihydroxytetrahydrofuran- 2- yl)methyl sulfamate | 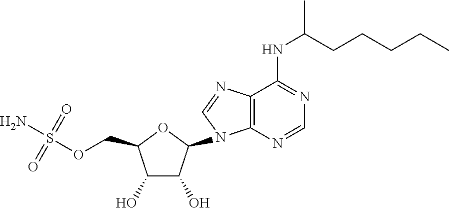 [SMILES: CCCCCC(C)Nc1ncnc2c1ncn2[C@@H]1O[C@H](COS(N)(=O)=O)[C@@H](O)[C@H]1O] |
| Compound 20 | ((2R,3S,4R,5R)- 3,4-dihydroxy-5- (6-((2-methoxyethyl) amino)-9H- purin-9- yl)tetrahydrofuran-2- yl)methyl sulfamate | 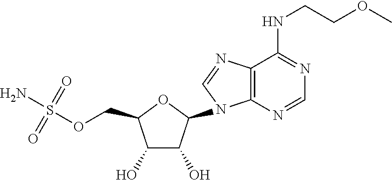 [SMILES: COCCNc1ncnc2c1ncn2[C@@H]1O[C@H](COS(N)(=O)=O)[C@@H](O)[C@H]1O] |
| Compound 21 | ((2R,3S,4R,5R)- 3,4-dihydroxy-5- (6-((1-methoxypropan-2- yl)amino)-9H-purin-9- yl)tetrahydrofuran- 2-yl)methyl sulfamate | 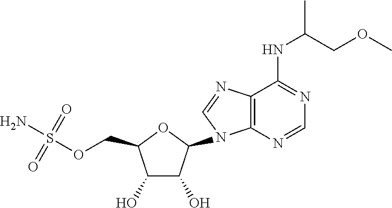 [SMILES: COCC(C)Nc1ncnc2c1ncn2[C@@H]1O[C@H](COS(N)(=O)=O)[C@@H](O)[C@H]1O] |
| Compound 22 | ((2R,3S,4R,5R)-5-(6-((2- (dimethylamino) ethyl)amino)-9H- purin-9-yl)-3,4- dihydroxytetrahydrofuran- 2- yl)methyl sulfamate | 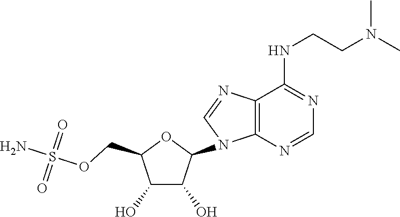 [SMILES: CN(C)CCNc1ncnc2c1ncn2[C@@H]1O[C@H](COS(N)(=O)=O)[C@@H](O)[C@H]1O] |
| Compound 23 | ((2R,3S,4R,5R)-5-(6- (butyl(methyl)amino)- 9H-purin-9- yl)-3,4- dihydroxytetrahydrofuran- 2-yl)methyl sulfamate | 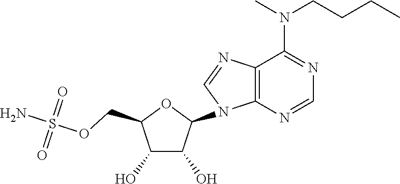 [SMILES: CCCCN(C)c1ncnc2c1ncn2[C@@H]1O[C@H](COS(N)(=O)=O)[C@@H](O)[C@H]1O] |
| Compound 24 | ((2R,3S,4R,5R)-5-(6- (diethylamino)- 9H-purin-9-yl)- 3,4- dihydroxytetrahydrofuran-2- yl)methyl sulfamate | 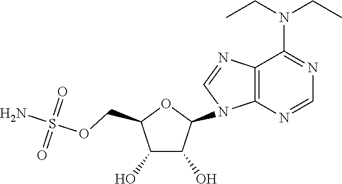 [SMILES: CCN(CC)c1ncnc2c1ncn2[C@@H]1O[C@H](COS(N)(=O)=O)[C@@H](O)[C@H]1O] |
| Compound 25 | ((2R,3S,4R,5R)-5-(6- (ethyl(isopropyl) amino)-9H- purin-9-yl)-3,4- dihydroxytetrahydrofuran- 2- yl)methyl sulfamate | 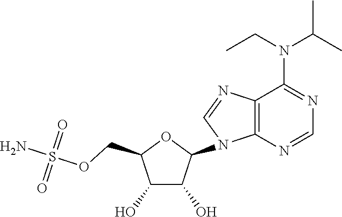 [SMILES: CCN(c1ncnc2c1ncn2[C@@H]1O[C@H](COS(N)(=O)=O)[C@@H](O)[C@H]1O)C(C)C] |
| Compound 26 | ((2R,3S,4R,5R)-5-(6- (butyl(ethyl)amino)- 9H-purin-9- yl)-3,4- dihydroxytetrahydrofuran- 2-yl)methyl sulfamate | 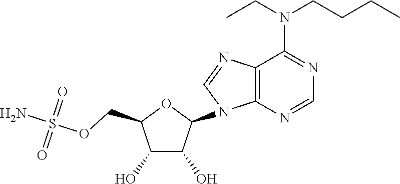 [SMILES: CCCCN(CC)c1ncnc2c1ncn2[C@@H]1O[C@H](COS(N)(=O)=O)[C@@H](O)[C@H]1O] |
| Compound 27 | ((2R,3S,4R,5R)-5-(6- (dibutylamino)- 9H-purin-9-yl)- 3,4- dihydroxytetrahydrofuran- 2- yl)methyl sulfamate | 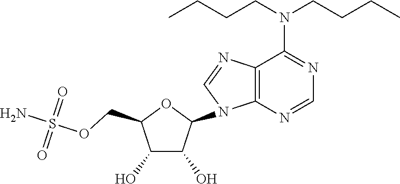 [SMILES: CCCCN(CCCC)c1ncnc2c1ncn2[C@@H]1O[C@H](COS(N)(=O)=O)[C@@H](O)[C@H]1O] |
| Compound 28 | ((2R,3S,4R,5R)-5-(6- (cyclopentylamino)- 9H-purin-9- yl)-3,4- dihydroxytetrahydrofuran- 2-yl)methyl sulfamate | 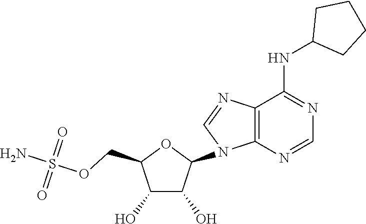 [SMILES: NS(=O)(=O)OC[C@H]1O[C@@H](n2cnc3c(NC4CCCC4)ncnc32)[C@H](O)[C@@H]1O] |
| Compound 29 | ((2R,3S,4R,5R)- 3,4-dihydroxy-5- (6-(((tetrahydrofuran-2- yl)methyl)amino)- 9H-purin-9- yl)tetrahydrofuran- 2-yl)methyl sulfamate | 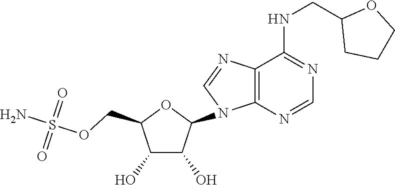 [SMILES: NS(=O)(=O)OC[C@H]1O[C@@H](n2cnc3c(NCC4CCCO4)ncnc32)[C@H](O)[C@@H]1O] |
| Compound 30 | ((2R,3S,4R,5R)- 5-(6-((furan-2- ylmethyl)amino)- 9H-purin-9-yl)- 3,4- dihydroxytetrahydrofuran- 2- yl)methyl sulfamate | 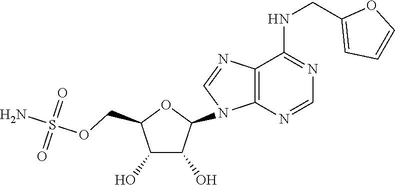 [SMILES: NS(=O)(=O)OC[C@H]1O[C@@H](n2cnc3c(NCc4ccco4)ncnc32)[C@H](O)[C@@H]1O] |
| Compound 31 | ((2R,3S,4R,5R)- 5-(6-((furan-2- ylmethyl)(methyl) amino)-9H- purin-9-yl)-3,4- dihydroxytetrahydrofuran- 2- yl)methyl sulfamate | 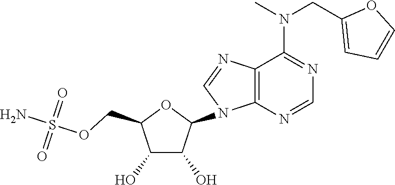 [SMILES: CN(Cc1ccco1)c1ncnc2c1ncn2[C@@H]1O[C@H](COS(N)(=O)=O)[C@@H](O)[C@H]1O] |
| Compound 32 | ((2R,3S,4R,5R)- 3,4-dihydroxy-5- (6-((thiophen-2- ylmethyl)amino)- 9H-purin-9-yl) tetrahydrofuran-2- yl)methyl sulfamate | 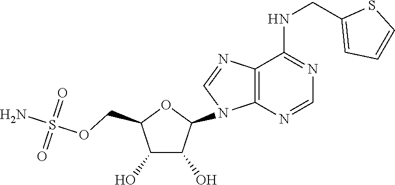 [SMILES: NS(=O)(=O)OC[C@H]1O[C@@H](n2cnc3c(NCc4cccs4)ncnc32)[C@H](O)[C@@H]1O] |
| Compound 33 | ((2R,3S,4R,5R)- 3,4-dihydroxy-5- (6-((2-(thiophen-2- yl)ethyl)amino)- 9H-purin-9- yl)tetrahydrofuran- 2-yl)methyl sulfamate | 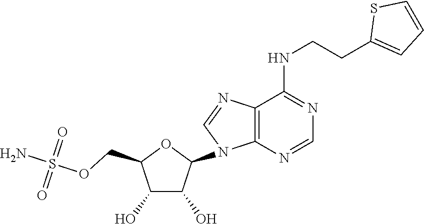 [SMILES: NS(=O)(=O)OC[C@H]1O[C@@H](n2cnc3c(NCCc4cccs4)ncnc32)[C@H](O)[C@@H]1O] |
| Compound 34 | ((2R,3S,4R,5R)- 5-(6-((3-(1H- imidazol-1-yl) propyl)amino)-9H- purin-9-yl)-3,4- dihydroxytetrahydrofuran-2- yl)methyl sulfamate | 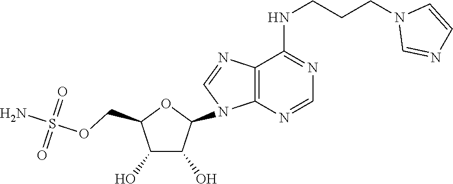 [SMILES: NS(=O)(=O)OC[C@H]1O[C@@H](n2cnc3c(NCCCn4ccnc4)ncnc32)[C@H](O)[C@@H]1O] |
| Compound 35 | ((2R,3S,4R,5R)-5-(6- (cyclohexylamino)- 9H-purin-9- yl)-3,4- dihydroxytetrahydrofuran- 2-yl)methyl sulfamate | 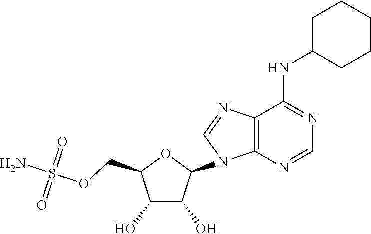 [SMILES: NS(=O)(=O)OC[C@H]1O[C@@H](n2cnc3c(NC4CCCCC4)ncnc32)[C@H](O)[C@@H]1O] |
| Compound 36 | ((2R,3S,4R,5R)- 5-(6-(((1S,2R)- bicyclo[2.2.1] heptan-2-yl)amino)- 9H-purin-9-yl)-3,4- dihydroxytetrahydrofuran-2- yl)methyl sulfamate | 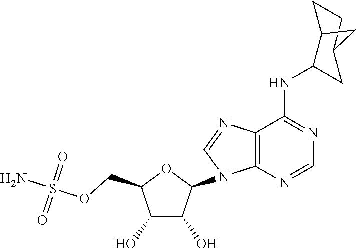 [SMILES: NS(=O)(=O)OC[C@H]1O[C@@H](n2cnc3c(NC4CC5CCC4C5)ncnc32)[C@H](O)[C@@H]1O] |
| Compound 37 | ((2R,3S,4R,5R)-5-(6- ((cyclohexylmethyl) amino)-9H- purin-9-yl)-3,4- dihydroxytetrahydrofuran- 2- yl)methyl sulfamate | 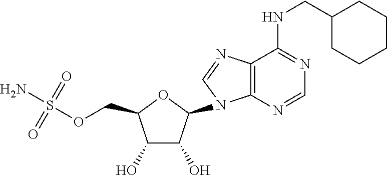 [SMILES: NS(=O)(=O)OC[C@H]1O[C@@H](n2cnc3c(NCC4CCCCC4)ncnc32)[C@H](O)[C@@H]1O] |
| Compound 38 | ((2R,3S,4R,5R)- 3,4-dihydroxy-5- (6-(phenylamino)- 9H-purin-9- yl)tetrahydrofuran- 2-yl)methyl sulfamate | 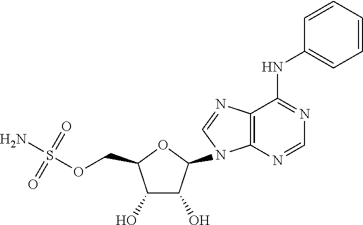 [SMILES: NS(=O)(=O)OC[C@H]1O[C@@H](n2cnc3c(Nc4ccccc4)ncnc32)[C@H](O)[C@@H]1O] |
| Compound 39 | ((2R,3S,4R,5R)- 3,4-dihydroxy-5- (6-((tetrahydro- 2H-pyran-4- yl)amino)-9H-purin-9- yl)tetrahydrofuran- 2-yl)methyl sulfamate | 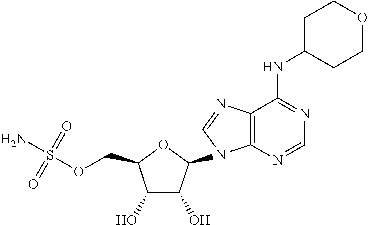 [SMILES: NS(=O)(=O)OC[C@H]1O[C@@H](n2cnc3c(NC4CCOCC4)ncnc32)[C@H](O)[C@@H]1O] |
| Compound 40 | ((2R,3S,4R,5R)- 3,4-dihydroxy-5- (6-((pyridin-2- ylmethyl)amino)- 9H-purin-9- yl)tetrahydrofuran-2- yl)methyl sulfamate | 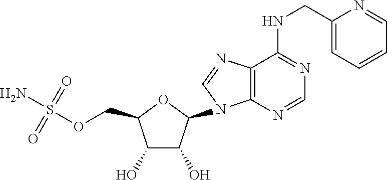 [SMILES: NS(=O)(=O)OC[C@H]1O[C@@H](n2cnc3c(NCc4ccccn4)ncnc32)[C@H](O)[C@@H]1O] |
| Compound 41 | ((2R,3S,4R,5R)- 3,4-dihydroxy-5- (6-((pyridin-3- ylmethyl)amino)- 9H-purin-9-yl) tetrahydrofuran-2- yl)methyl sulfamate | 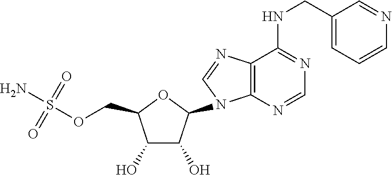 [SMILES: NS(=O)(=O)OC[C@H]1O[C@@H](n2cnc3c(NCc4cccnc4)ncnc32)[C@H](O)[C@@H]1O] |
| Compound 42 | ((2R,3S,4R,5R)- 3,4-dihydroxy-5- (6-((pyridin-4- ylmethyl)amino)- 9H-purin-9-yl) tetrahydrofuran-2- yl)methyl sulfamate | 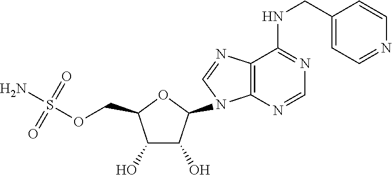 [SMILES: NS(=O)(=O)OC[C@H]1O[C@@H](n2cnc3c(NCc4ccncc4)ncnc32)[C@H](O)[C@@H]1O] |
| Compound 43 | ((2R,3S,4R,5R)-5-(6- (benzyl(methyl) amino)-9H-purin- 9-yl)-3,4- dihydroxytetrahydrofuran- 2- yl)methyl sulfamate | 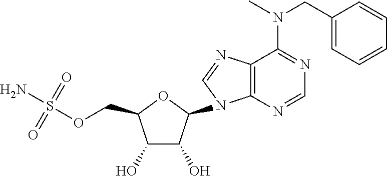 [SMILES: CN(Cc1ccccc1)c1ncnc2c1ncn2[C@@H]1O[C@H](COS(N)(=O)=O)[C@@H](O)[C@H]1O] |
| Compound 44 | ((2R,3S,4R,5R)- 3,4-dihydroxy-5- (6-morpholino- 9H-purin-9- yl)tetrahydrofuran- 2-yl)methyl sulfamate | 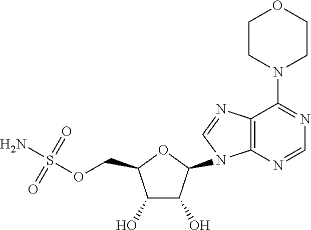 [SMILES: NS(=O)(=O)OC[C@H]1O[C@@H](n2cnc3c(N4CCOCC4)ncnc32)[C@H](O)[C@@H]1O] |
| Compound 45 | ((2R,3S,4R,5R)-5-(6- (cycloheptylamino)- 9H-purin-9- yl)-3,4- dihydroxytetrahydrofuran- 2-yl)methyl sulfamate | 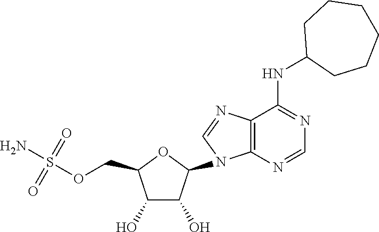 [SMILES: NS(=O)(=O)OC[C@H]1O[C@@H](n2cnc3c(NC4CCCCCC4)ncnc32)[C@H](O)[C@@H]1O] |
| Compound 46 | ((2R,3S,4R,5R)-5-(6- (cyclooctylamino)- 9H-purin-9-yl)- 3,4- dihydroxytetrahydrofuran- 2- yl)methyl sulfamate | 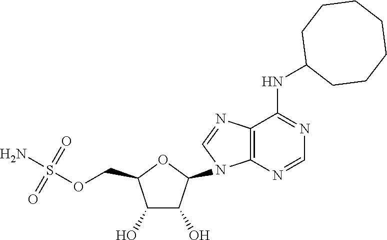 [SMILES: NS(=O)(=O)OC[C@H]1O[C@@H](n2cnc3c(NC4CCCCCCC4)ncnc32)[C@H](O)[C@@H]1O] |
| Compound 47 | ((2R,3S,4R,5R)- 3,4-dihydroxy-5- (6- (octahydroisoquinolin- 2(1H)- yl)-9H-purin-9- yl)tetrahydrofuran- 2-yl)methyl sulfamate | 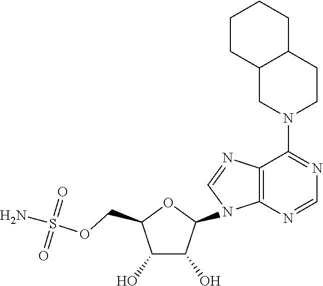 [SMILES: NS(=O)(=O)OC[C@H]1O[C@@H](n2cnc3c(N4CCC5CCCCC5C4)ncnc32)[C@H](O)[C@@H]1O] |
| Compound 48 | ((2R,3S,4R,5R)-5-(6- (((1,4,7,10,13- pentaoxacyclopentadecan- 2- yl)methyl)amino)- 9H-purin-9-yl)- 3,4- dihydroxytetrahydrofuran- 2- yl)methyl sulfamate | 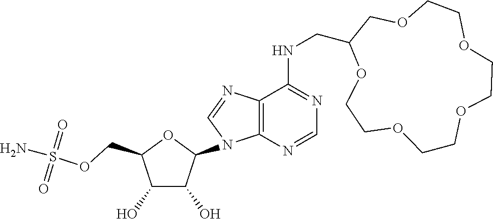 [SMILES: NS(=O)(=O)OC[C@H]1O[C@@H](n2cnc3c(NCC4COCCOCCOCCOCCO4)ncnc32)[C@H](O)[C@@H]1O] |
| ABP A3 Compound 49 | ((2R,3S,4R, 5R)-5-(6-((3- ethynylphenyl)amino)- 9H-purin- 9-yl)-3,4- dihydroxytetrahydrofuran-2- yl)methyl sulfamate | 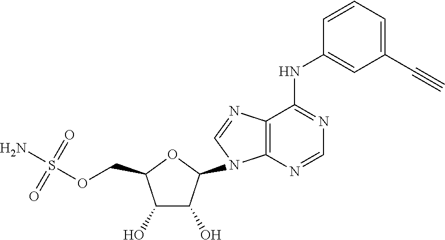 [SMILES: C#Cc1cccc(Nc2ncnc3c2ncn3[C@@H]2O[C@H](COS(N)(=O)=O)[C@@H](O)[C@H]2O)c1] |
| LZ3 Compound 50 | (Z)-3-(5-(2- methoxybenzylidene)- 2,4- dioxothiazolidin- 3-yl)-N-(4- sulfamoylphenyl) propanamide | 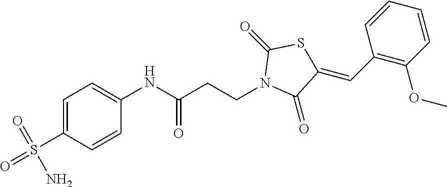 [SMILES: COc1ccccc1/C=C1\SC(=O)N(CCC(=O)Nc2ccc(S(N)(=O)=O)cc2)C1=O] |
| 6,6″- Biapigenin Compound 51 | 4′,5,7,7′-tetrahydroxy- 2,2′-bis(4- hydroxyphenyl)- 4H,5′H-[6,6′- bichromene]-4,5′-dione | 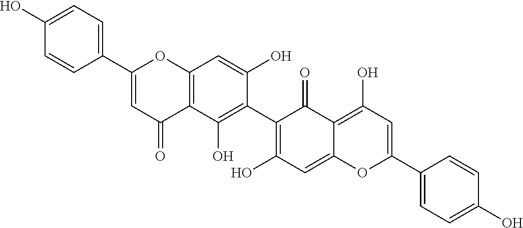 [SMILES: O=c1c2c(O)cc(-c3ccc(O)cc3)oc-2cc(O)c1-c1c(O)cc2oc(-c3ccc(O)cc3)cc(=O)c2c1O] |
| Deoxyvasicin one derivative Compound 52 | (2-((9-oxo-1,2,3,9- tetrahydropyrrolo[2,1- b]quinazolin-7- yl)oxy)acetyl) glycylglycine | 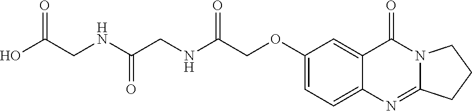 [SMILES: O=C(O)CNC(=O)CNC(=O)COc1ccc2nc3n(c(=O)c2c1)CCC3] |
| Flavokawain A Compound 53 | (E)-1-(2-hydroxy-4,6- dimethoxyphenyl)-3-(4- methoxyphenyl)prop- 2-en-1-one | 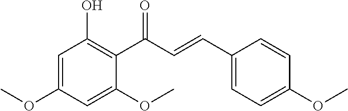 |
| [Rh(ppy)2(dp pz)] + Compound 54 | N/A | 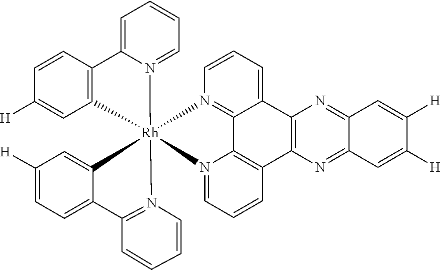 |
| [Rh(ppy)2(dp pz)] + Compound 55 | N/A | 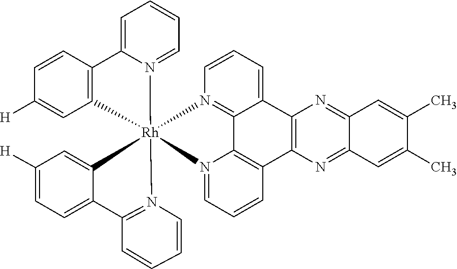 |
| [Rh(ppy)2(dp pz)] + Compound 56 | N/A | 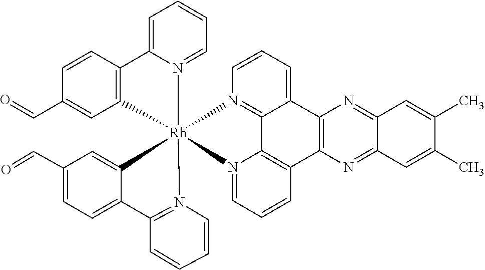 |
| [Rh(ppy)2(dp pz)] + Compound 57 | N/A | 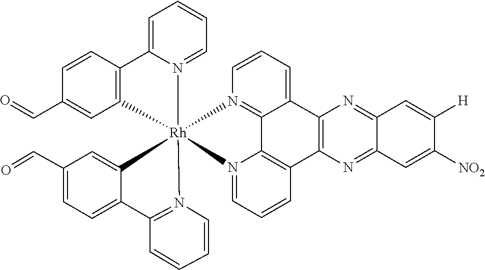 |
| [Rh(phq)2(M OPIP)] + Compound 58 | N/A | 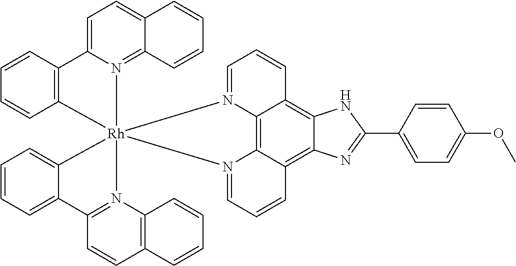 |
| Piperacillin Compound 59 | (2S,5R,6R)-6- ((R)-2-(4-ethyl-2,3- dioxopiperazine-1- carboxamido)-2- phenylacetamido)- 3,3-dimethyl- 7-oxo-4-thia-1- azabicyclo[3.2.0] heptane-2- carboxylate | 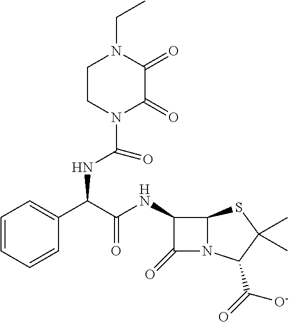 [SMILES: CCN1CCN(C(=O)N[C@@H](C(=O)N[C@@H]2C(=O)N3[C@@H]2SC(C)(C)[C@@H]3C(=O)[O-])c2ccccc2)C(=O)C1=O] |
| Mitoxantrone Compound 60 | 1,4-dihydroxy- 5,8-bis((2-((2- hydroxyethyl) amino)ethyl)amino) anthracene-9,10-dione | 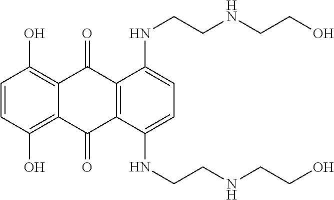 [SMILES: O=C1c2c(O)ccc(O)c2C(=O)c2c(NCCNCCO)ccc(NCCNCCO)c21] |
| M22 Compound 61 | 1-benzyl-N-(2,4- dichlorophenethyl) piperidin-4- amine | 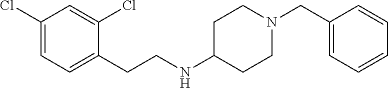 [SMILES: Clc1ccc(CCNC2CCN(Cc3ccccc3)CC2)c(Cl)c1] |
| LP0040 Compound 62 | 4-(3-methyl-4- (trifluoromethyl) phenyl)-7-(4- (phenethylamino) piperidin-1-yl)- 2H-chromen-2-one | 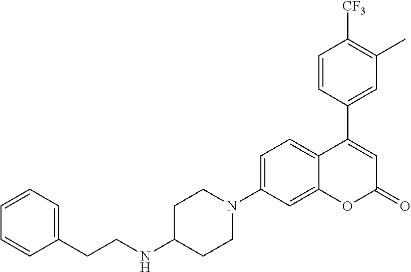 [SMILES: Cc1cc(-c2cc(=O)oc3cc(N4CCC(NCCc5ccccc5)CC4)ccc23)ccc1C(F)(F)F] |
| Compound 63 | 4-([1,1′-biphenyl]- 4-yl)-7-(4- (phenethylamino) piperidin-1-yl)- 2H-chromen-2-one | 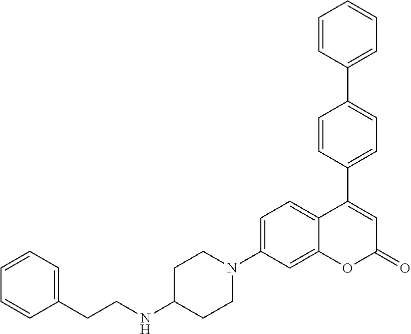 [SMILES: O=c1cc(-c2ccc(-c3ccccc3)cc2)c2ccc(N3CCC(NCCc4ccccc4)CC3)cc2o1] |
| Compound 64 | 7-(4-(phenethylamino) piperidin- 1-yl)-4- (4-phenoxyphenyl)-2H- chromen-2-one | 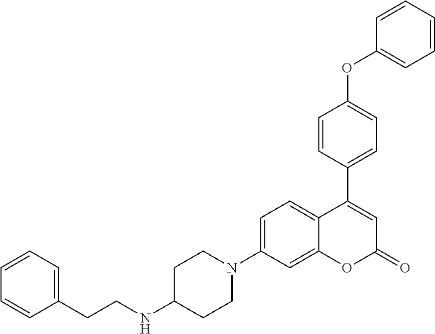 [SMILES: O=c1cc(-c2ccc(Oc3ccccc3)cc2)c2ccc(N3CCC(NCCc4ccccc4)CC3)cc2o1] |
| ZM223 Compound 65 | N-(6-(2-((4- aminophenyl)thio) acetamido) benzo[d]thiazol-2-yl)-4- (trifluoromethyl) benzamide | 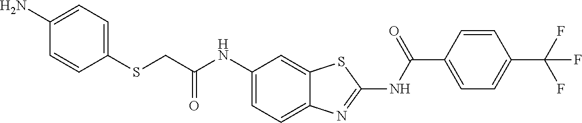 [SMILES: Nc1ccc(SCC(=O)Nc2ccc3nc(NC(=O)c4ccc(C(F)(F)F)cc4)sc3c2)cc1] |
| NaCM-OPT Compound 66 | 1-benzyl-1-(1- butylpiperidin-4- yl)-3-(3,4- dichlorophenyl)urea | 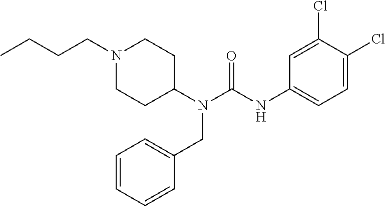 [SMILES: CCCCN1CCC(N(Cc2ccccc2)C(=O)Nc2ccc(Cl)c(Cl)c2)CC1] |

In some embodiments, the one or more NAE inhibitors are selected from MLN4924, TAS4464 and ZM223 (compounds 1, 2 and 65, respectively), or a salt, solvate and/or prodrug thereof.

In some embodiments, the NAE inhibitor is a covalent NAE inhibitor which binds covalently and essentially irreversibly with a component of the NAE complex and thereby inhibits NAE activity. In some embodiments, the NAE inhibitor is a non-covalent inhibitor which reversibly binds to the NAE complex and thereby inhibits NAE activity.

Also, the present application includes a composition comprising a compound of the application and one or more of a) a virus, suitably an attenuated virus, a genetically modified virus, a non-replicating gene therapy vector, or an oncolytic virus; b) one or more cancer cells; c) a carrier, diluent or excipient; d) a pharmaceutically acceptable carrier, diluent or excipient; e) non-cancer cells; f) cell culture media; or g) one or more cancer therapeutics; or any combination of a)-g).

In some embodiments, which is not meant to be limiting in any manner, included is a compound of the application and a medium for growing, culturing or infecting cells with a virus and optionally, one or more cells which are capable of being infected by the virus. In a further embodiment, the cells are immortalized cells, cancer cells or tumor cells. In an alternate embodiment, the cells are MDCK, HEK293, Vero, HeLa or PER.C6 cells.

In some embodiments, the salt is an acid addition salt or a base addition salt. In some embodiments, for pharmaceutical methods and uses on human or animal subjects, the salt is a pharmaceutically acceptable salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. The compounds of the application also include those in which one or more radioactive atoms are incorporated within their structure.

In some embodiments, the compounds of the present application are suitably formulated in a conventional manner into compositions using one or more carriers, optionally in combination with one or more viruses. Accordingly, the present application also includes a composition comprising one or more compounds of the application and a carrier. The present application also includes a composition comprising one or more compounds of the application, one or more viruses and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier as well as a pharmaceutical composition comprising one or more compounds of the application, one or more viruses and a pharmaceutically acceptable carrier. In embodiments of the application the pharmaceutical compositions are used in the treatment of any of the diseases, disorders or conditions described herein.

The compositions of the application are administered to a subject in a variety of forms depending on the selected route of administration, as will be understood by those skilled in the art. For example, a composition of the application is formulated for administration by oral, inhalation, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a composition of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compositions of the application and use the lyophilizates obtained, for example, for the preparation of products for injection.

In some embodiments, a composition of the application is administered parenterally. For example, solutions are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a composition of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form must be sterile and must be fluid to the extent that easy syringability exists. Alternatively, the compositions of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compositions of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active components. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges and pastilles, wherein a composition of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compositions of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compositions of the application are particularly amenable to administration with the aid of nano-carrier systems, such as liposomes, micelles, nanoparticles, nano-emulsions, lipidic nano-systems and the like (see for example, Bhat, M. et al. Chem. and Phys. of Lipids, 2021, 236, 105053). Accordingly the present application includes a composition comprising one or more compounds of the application, optionally one or more viruses and one or more components of a nano-carrier system.

Depending on the mode of administration, a pharmaceutical composition will comprise from about 0.05 wt % to about 99 wt % or about 0.10 wt % to about 70 wt %, of the active ingredient (compound(s) of the application and optionally one or more viruses) and from about 1 wt % to about 99.95 wt % or about 30 wt % to about 99.90 wt % of a pharmaceutically acceptable carrier, all percentages by weight being based on the total composition.

In some embodiments, the compositions of the application comprise an additional therapeutic agent. Therefore the present application also includes a pharmaceutical composition comprising one of more compounds of the application, optionally one or more viruses and an additional therapeutic agent, and optionally one or more pharmaceutically acceptable excipients. In some embodiments, the additional therapeutic agent is an anticancer drug.

Also included is a kit comprising the compound of the application and a) a virus, suitably an attenuated or genetically modified virus or an oncolytic virus; b) one or more cancer cells; c) a pharmaceutically acceptable carrier, diluent or excipient; d) non-cancer cells; e) cell culture media; f) one or more cancer therapeutics, g) a cell culture plate or multi-well dish; h) an apparatus to deliver the viral sensitizing compound to a cell, medium or to a subject; i) instructions for using the viral sensitizing agent; or j) a carrier diluent or excipient, or any combination of a)-j).

In some embodiments, which are not meant to be limiting in any manner, included is a kit comprising a compound of the application and a medium for growing, culturing or infecting cells with a virus and optionally, one or more cells which are capable of being infected by the virus.

In some embodiments, the kit comprises instructions for using any component or combination of components and/or practicing any method as described herein.

In the above, the term “a compound” also includes embodiments wherein one or more compounds are referenced.

### III. Methods and Uses of the Application

The application also provides uses and methods relating to the compounds and compositions described herein.

The compounds and compositions of the application have been shown to increase permissiveness of a cell to a virus, and thus exhibit viral sensitizing activity, with high potency and versatility. Accordingly, the compounds and compositions of the application are useful for increasing permissiveness of a cell to a virus and for treating diseases, disorders or conditions by increasing permissiveness of a cell to a virus. The compounds and compositions of the application have also been shown to enhance virus production by a cell, for example in a reverse genetics system. Accordingly, compounds and compositions of the application are also useful for increasing virus production. The compounds and compositions of the application are also useful for increasing the oncolytic activity of a virus, treating a disease, disorder or condition by gene therapy, increasing transduction, increasing virally-encoded transgene expression and increasing virus growth and/or spread. In some embodiments the cell is in vitro. In some embodiments, the cell is ex vivo. In some embodiments, the cells is in vivo (i.e. in a subject).

In some embodiments, the virus is a therapeutic virus.

In some embodiments, the virus is an interferon (IFN)-sensitive virus.

In some embodiments, the virus is an attenuated virus, a genetically modified virus, a non-replicating virus, or an oncolytic virus.

In some embodiments, the virus is a non-replicating viral vector, optionally an adenovirus (Ad), an adeno-associated virus (AAV) or lentivirus (LV).

In some, embodiments, the virus is a herpes simplex virus (HSV) viral vector.

In some embodiments, the virus is a gene therapy vector. As used herein, the term “gene therapy vector” is used to refer to a viral vector designed to deliver therapeutic genetic material to a cell or subject. Examples of gene therapy vectors include, but are not limited to human Ad5, Ad3, Ad11, Ad35, canine Ad2, chimp Ad26, chimp AdOx1, or recombinant serotypes therein, AAV serotypes 1-9 or recombinant serotypes therein, Lentivirus, gamma-retrovirus, Annellovirus, or Baculovirus.

In some embodiments, the virus is a component of a vaccine. For example but not limited to a live attenuated vaccine such as, measles, mumps, rubella, rotavirus, chickenpox, yellow fever or a viral vector vaccine encoding a vaccine antigen transgene such as rVSVΔG-ZEBOV-GP (Ervebo) or ChadOx1-S (Vaxzevria).

In some embodiments, the virus is a rhabdovirus, a togavirus, or an orthomyxovirus.

In some embodiments, the rhabdovirus is vesicular stomatitis virus (VSV), engineered mutants of VSV (VSVΔ51), an oncolytic non-VSV rhabdovirus, or a recombinant oncolytic non-VSV rhabdovirus encoding one or more of rhabdoviral N, P, M, G and/or L protein, or variant thereof including chimeras and fusion proteins thereof, having an amino acid identity of at least or at most 20, 30, 40, 50, 60, 65, 70, 75, 80, 85, 90, 92, 94, 96, 98, 99, 100%, including all ranges and percentages there between, to the N, P, M, G and/or L protein of Arajas virus, Chandipura virus, Cocal virus, Isfahan virus, Maraba virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus.

In some embodiments, the togavirus is sindbis, semliki forest virus or M1 virus.

In some embodiments, the orthomyxovirus is influenza A, influenza B, influenza C, influenza D, isavirus, thogotovirus or quanranjavirus.

### Methods and Uses of Increasing Permissiveness of a Cell to a Virus

The present application includes a method of increasing permissiveness of a cell or a subject to a virus, comprising administering an effective amount of a neddylation-activating enzyme (NAE) inhibitor, or a salt, solvate and/or prodrug thereof, (i.e. a compound of the application) to the cell or the subject.

Also provided is use of a compound of the application to increase permissiveness of a cell or a subject to a virus. In another embodiment, a compound of the application is used in the manufacture of a medicament to increase permissiveness of a cell or a subject to a virus. In yet another embodiment, a compound of the application is for use in increasing permissiveness of a cell or a subject to a virus.

In some embodiments, the compound of the application is administered to the cell before, after and/or concurrently with the virus. In some embodiments, the compound of the application is administered to the cell before the virus is administered to the cell.

In some embodiments, permissiveness of the cell to the virus is increased 1.1 fold or more, 1.2 fold or more, 1.5 fold or more, 2 fold or more, 2.5 fold or more, 3 fold or more, 5 fold or more, or 10 fold or more, e.g., compared to permissiveness of the cell, or a comparable cell prior to the method or in the absence of the method. In some cases, the method includes measuring the increase in permissiveness to viral infection.

In some embodiments, the cell is a eukaryotic cell, for example a human or other mammalian cell. In some embodiments, the cell is a prokaryotic cell.

The cell is optionally in vivo, ex vivo or in vitro. In some embodiments, the cell is a cell in subject (i.e. in vivo). In some embodiments, the cell is a cell in vitro, for example a cell line or a cell culture.

### Methods and Uses of Increasing Permissiveness of a Cell to Genetic Material Encoding Components of a Virus

The present application includes a method of increasing permissiveness of a cell to genetic material encoding components of a virus, comprising administering an effective amount of a neddylation-activating enzyme (NAE) inhibitor, or a salt, solvate and/or prodrug thereof, (i.e. a compound of the application) to the cell prior, concurrent with, or after provision of the said genetic material to the cell using a carrier or method commonly known to a person skilled in the art. In no way limiting, this includes transfection using PEI or lipid-based reagents, electroporation, and nanoparticles, as is generally known in the art.

Also provided is use of a compound of the application prior, concurrent with, or after provision of genetic material to a cell using a carrier to increase permissiveness of a cell to genetic material encoding components of a virus.

The term “genetic material encoding components of a virus” refers to nucleic acids, and chemically modified variants thereof, that comprise or consist of viral and/or viral-like sequences. The sequences can encode viral proteins, viral-like proteins and/or functional sequences for targeting, integration, promotion, etc.

Delivery of such genetic material to a cell is generally described as transfection. Transfection efficiency refers to the degree to which a supplied source of genetic material is taken up and functional for its intended purpose in a cell. In some embodiments, the provision comprises delivery of such genetic material to a cell is generally described as transfection.

Such genetic material may be delivered to the cell directly, or it may be provided in a carrier to enhance delivery and uptake by the cell. Examples of carriers include diverse polymers known in the art, which may be designed in a variety of nanoparticle formats, either loosely organized or more precision designed. Common carriers of genetic material are described, for example, in Cullis and Hope (2017) and in Mitchell et al. (2021).

In some embodiments, the genetic material is comprised in a plasmid.

In one example, lentivirus, gamma-Retrovirus, or AAV are produced following transfection of plasmids encoding lentivirus, gamma-Retrovirus, or AAV viral or viral-like sequences into a cell.

### Methods and Uses of Treating Diseases, Disorders or Conditions

Compounds and compositions of the application are useful for treating diseases, disorders or conditions by increasing permissiveness of a cell to a virus. Therefore, the compounds and compositions of the present application are useful as medicaments and the application also includes a compound or composition of the application for use as a medicament in combination with a virus.

Accordingly, the present application includes a method of treating a disease, disorder or condition by increasing permissiveness of a cell to a virus comprising administering a therapeutically effective amount of a compound of the application and the virus or genetic material encoding the virus to a subject in need thereof. Also provided is use of a compound of the application and a virus or genetic material encoding a virus to treat a disease, disorder or condition. In another embodiment, a compound of the application is used in the manufacture of a medicament for treating a disease, disorder or condition in combination with a virus or genetic material encoding a virus.

In some embodiments, the compound of the application is administered to the cell before, after and/or concurrently with a virus that treats the disease, disorder or condition or genetic material encoding a virus that treats the disease, disorder or condition. In further embodiment, the compound of the application is for use before, after and/or concurrently with use of a virus that treats the disease, disorder or condition or genetic material encoding a virus that treats the disease, disorder or condition. In yet another embodiment, a compound of the application is for use before, after and/or concurrently with use of a virus that treats the disease, disorder or condition or genetic material encoding a virus that treats the disease, disorder or condition.

In some embodiments, the compound of the application allows a lower amount of the virus to be used to treat the disease, disorder or condition and/or increases the therapeutic efficacy of the virus.

In some embodiments, the virus is a therapeutic virus, for example a gene therapy vector.

In some embodiments, the disease, disorder or condition is cancer or tumor. In such embodiments, the virus is optionally an oncolytic virus. As used herein, an oncolytic virus is a virus that preferentially infects and lyses cancer or tumor cells as compared to non-cancer or normal cells. Oncolytic viruses can be natural or engineered.

In some embodiment, the cancer is a tumor.

In some embodiments, the cancer is lymphoblastic leukemia, myeloid leukemia, adrenocortical carcinoma, AIDS-related cancer, AIDS-related lymphoma, anal cancer, appendix cancer, astrocytoma, atypical teratoid/rhabdoid tumor, basal cell carcinoma, bile duct cancer, bladder cancer, bone cancer, osteosarcoma, malignant fibrous histiocytoma, brain stem glioma, brain tumor, cerebellar astrocytoma, cerebral astrocytoma/malignant glioma, craniopharyngioma, ependymoblastoma, medulloblastoma, pineal parenchymal tumors of intermediate differentiation, supratentorial primitive neuroectodermal tumors and pineoblastoma, visual pathway and hypothalamic glioma, spinal cord tumors, breast cancer, bronchial tumors, Burkitt lymphoma, carcinoid tumor, central nervous system lymphoma, cervical cancer, chordoma, chronic lymphocytic leukemia, chronic myelogenous leukemia, chronic myeloproliferative disorders, colon cancer, cutaneous T-Cell lymphoma, embryonal tumors, endometrial cancer, ependymoblastoma, ependymoma, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), gastrointestinal stromal cell tumor, germ cell tumors, extracranial, extragonadal, ovarian, gestational trophoblastic tumor, glioma, hairy cell leukemia, head and neck cancer, hepatocellular (Liver) cancer, histiocytosis, Langerhans cell cancer, Hodgkin lymphoma, hypopharyngeal cancer, islet cell tumors, Kaposi sarcoma, kidney cancer, laryngeal cancer, lymphocytic leukemia, hairy cell leukemia, lip and oral cavity cancer, liver cancer, non-small cell lung cancer, small cell lung cancer, Hodgkin lymphoma, non-Hodgkin lymphoma, malignant fibrous hi stiocytoma of bone and osteosarcoma, medulloblastoma, medulloepithelioma, melanoma, intraocular melanoma, Merkel cell carcinoma, mesothelioma, metastatic squamous neck cancer, mouth cancer, multiple endocrine neoplasia syndrome, multiple myeloma/plasma cell neoplasm, nasal cavity and paranasal sinus cancer, nasopharyngeal cancer, neuroblastoma, oral cancer, oropharyngeal cancer, ovarian cancer, pancreatic cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, renal pelvis and ureter cancer, transitional cell cancer, respiratory tract carcinoma, retinoblastoma, rhabdomyosarcoma, salivary gland cancer, uterine sarcoma, skin cancer, Merkel cell skin carcinoma, small intestine cancer, soft tissue sarcoma, squamous cell carcinoma, squamous neck cancer, stomach (Gastric) cancer, supratentorial primitive neuroectodermal tumors, T-Cell lymphoma, testicular cancer, throat cancer, thymoma and thymic carcinoma, thyroid cancer, trophoblastic tumor, urethral cancer, uterine cancer, endometrial cancer, uterine sarcoma, vaginal cancer, vulvar cancer, or Wilms tumor.

In some embodiments, the cancer is colon cancer, breast cancer, rectal cancer, lung cancer, a leukemia, cervical cancer, sarcoma, melanoma, pancreatic cancer and/or ovarian cancer.

In some embodiments, the oncolytic virus is talimogene laherparepvec (T-VEC), Delytact, Maraba MG-1, or vesicular stomatitis virus (VSVΔ51). In some embodiments, the oncolytic virus is a Newcastle Disease Virus (NDV), measles virus, (MeV), parvovirus H1 (ParvOryx), M1 virus, poliovirus, reovirus, Myxomavirus, or Sindbis virus (SinV).

In some embodiments, the subject is a mammal. In another embodiment, the subject is human.

In some embodiments, the cell is a cancer cell, a tumor cell or an immortalized cell. In some embodiments, the cells are cancer cells or tumor cells in vivo, or in vitro. In some embodiments, the cell is one or more types of immortalized cells in vitro or in vivo from any cell, cell line, tissue or organism, not limited to, human, rat, mouse, cat, dog, pig, primate, horse and the like, for example, without limitation: Vero, HEK-293 cells, VPC 1.0, VPC 2.0, EB-66 cells, EbX cells, PER. C6 cells, AGE1.CR, Agel.0 S, Agel.HN, Agel.RO, Q0R2/2E11, UMNSAH-DF1, CHO, hybridoma cells, sf9 cells, or R⁴ cells. In some embodiments, the cell is cancer or tumor cells in vitro or in vivo from any cell, cell line, tissue or organism, for example, but not limited to human, rat, mouse, cat, dog, pig, primate, horse and the like, for example tumor forming cells such as, but not limited to 293-T cells, BHK21 cells, or MDCK cells, or cells and tumor cells from cancer and tumor listed in the application.

In some embodiments, the term “treatment” refers to beneficial or desired clinical results which can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

In some embodiments, effective amounts vary according to factors such as the disease state, age, sex and/or weight of the subject or species. In some embodiments, the amount of a given composition that will correspond to an effective amount will vary depending upon factors, such as the given drug(s), compound(s) and/or viruses, the pharmaceutical formulation, the route of administration, the schedule of administration, the type of condition, disease or disorder, the identity of the subject being treated and the like, but can nevertheless be routinely determined by one skilled in the art.

### Methods and Uses of Increasing Oncolytic Activity of a Virus

The present application also includes a method of increasing the oncolytic activity of a virus comprising administering a therapeutically effective amount of a compound of the application with an oncolytic virus to a subject or cell in need thereof. Also provided is use of a compound of the application for increasing the oncolytic activity of an oncolytic virus. In another embodiment, a compound of the application is used in the manufacture of a medicament for increasing the oncolytic activity of an oncolytic virus. In yet another embodiment, a compound of the application is for use in for increasing the oncolytic activity of an oncolytic virus.

As used herein, the expression “oncolytic activity” refers to the ability of the virus to infect and kill a cancer cell.

In some embodiments, the oncolytic activity of the virus is increased 1.1 fold or more, 1.2 fold or more, 1.5 fold or more, 2 fold or more, 2.5 fold or more, 3 fold or more, 5 fold or more, or 10 fold or more, e.g., compared to the oncolytic activity of the virus, or a comparable virus prior to the method or in the absence of the method. In some cases, the method includes measuring the increase in oncolytic activity of the virus.

### Methods and Uses of Treating a Disease, Disorder or Condition by Gene Therapy

The present application also includes a method of treating a disease, disorder or condition by gene therapy comprising administering a therapeutically effective amount of a compound of the application and a gene therapy vector to a subject or cell in need thereof. Gene therapy vectors are understood to be viral or non-viral, where non-viral may include genetic material encoding a virus as described elsewhere herein. Also included is use of a compound of the application for treating a disease, disorder or condition by gene therapy, wherein the compound is for use in combination with a gene therapy vector, as well as a use of a compound of the application in the manufacture of a medicament for treating a disease, disorder or condition by gene therapy, wherein the compound is for use in combination with a gene therapy vector. The application also includes, a compound of the application for use in treating a disease, disorder or condition by gene therapy, wherein the compound is for use in combination with a gene therapy vector.

In some embodiments, the gene therapy vector comprises a therapeutic gene for treating said disease, disorder or condition. In such an embodiment, the compound of the application increases permissiveness of the cell to the gene therapy vector to increase the amount of virally-encoded therapeutic gene incorporated into the cell. The therapeutic gene may be incorporated into the genome of the cell or may stay episomal.

### Methods and Uses of Increasing Production of Viruses

The application shows the ability of a compound of the application to enhance the production of a non-replicating AAV vector produced from a plasmid-transfection based reverse genetics system. Accordingly, the present application also includes a method, optionally an in vitro method, of increasing production of a virus by a cell comprising administering a compound of the application to the cell. Also included is a use of a compound of the application for increasing production of a virus by a cell, as well as a compound of the application for use in increasing production of a virus by a cell.

In some embodiments, the virus is produced from a plasmid-transfection based reverse genetics system. As known in the art, plasmid-transfection based reverse genetics systems generate a virus from viral proteins expressed from one or more plasmids in a cell.

Numerous reverse genetics systems are known, including for example the “AAV Helper Free System”. Other systems exist for example, for lentivirus, orthomyxovirus (influenza), coronavirus, baculovirus, retrovirus, rhabdovirus and paramyxovirus (NDV).

In some embodiments, the method comprises transfecting a cell with one or more plasmids encoding one or more components of a virus and contacting the transfected cell with a compound of the application to produce the virus. As is known in the art, once transfected into a cell, the one or more components of a virus can self-assemble into a virus.

As used herein, the terms “transfecting” and “transfection” refer to the delivery of genetic material (for example, plasmids) to a cell. Various methods of transfection are well known in art.

In some embodiments, the components of a virus are viral gene products, for example structural components of a virus. In some embodiments, the components of a virus comprise one or more genomic sequences.

In some embodiments, the plasmid encoding one or more components of a virus is a plasmid designed for use in a reverse genetics system. Examples of appropriate plasmids include, but are not limited to, pHelper, pAAV ITR-Fluc vector and pAAV Rep-Ca, all of which are known in the art.

In some embodiments, the method comprises co-transfecting a cell with pHelper, pAAV ITR-Fluc vector and pAAV Rep-C.

In some embodiments, “contacting the cell with a compound of the application” comprises growing the cell in an appropriate medium in the presence of a compound of the application. The cell is optionally contacted with a compound of the application before, after and/or during transfection.

A person of skill in the art will readily be able to determine the appropriate amount of compound to be used for contacting the cell and the duration of contact. In some embodiments, the cell is contacted with 100-800 nM of compound, optionally 200-400 nM of compound.

In some embodiments, the cell is a viral production cell, namely a cell that is used to produce a virus. Examples of viral production cells include, but are not limited to, Vero, HEK-293, VPC 1.0, VPC 2.0, EB-66, EbX, PER, C6, AGE1.CR, UMNSAH-DF1, CEF, MRC-5, WI-38, BHK21, Hela, A549 and sf9 cells.

In some embodiments, the virus produced by the cell is a non-replicating virus. In some embodiments, the virus produced by the cell is an oncolytic virus, gene therapy vector or a vaccine. In some embodiments, the virus produced by the cell is a non-replicating viral vector, optionally an adenovirus (Ad), an adeno-associated virus (AAV) or lentivirus (LV). In some embodiments, the virus is a non-replicating adeno-associated virus (AAV).

In some embodiments, production of the virus increased 1.1 fold or more, 1.2 fold or more, 1.5 fold or more, 2 fold or more, 2.5 fold or more, 3 fold or more, 5 fold or more, or 10 fold or more, e.g., compared to production of the virus by the cell, or a comparable cell, prior to the method or in the absence of the method. In some cases, the method includes measuring the increase in production of the virus, for example by determining the level of the virus in the cell and/or in the cell culture medium. Methods for determining virus production are known in the art and include, but are not limited to plaque assays, TCID50, PCR, ddPCR, ELISA, SRID and HPLC.

In some embodiments, the method comprises growing a replicating virus in an appropriate medium in the presence of a compound of the application.

### Methods and Uses of Increasing Transduction

The present application also includes a method, optionally an in vitro method, of increasing transduction of a virus into a cell comprising administering a compound of the application and the virus to the cell. Also included is a use of a compound of the application for increasing transduction of a virus into a cell, as well as a compound of the application for use in increasing transduction of a virus into a cell.

“Transduction” or “transducing” as used herein, refers to the introduction of a virus containing an exogenous gene into a cell leading to expression of the gene, e.g., the transgene in the cell. The gene is optionally a therapeutic gene.

As used herein, the expression “increasing transduction” includes increasing transduction efficiency.

In some embodiments, transduction of the virus is increased 1.1 fold or more, 1.2 fold or more, 1.5 fold or more, 2 fold or more, 2.5 fold or more, 3 fold or more, 5 fold or more, or 10 fold or more, e.g., compared to transduction of the virus by the cell, or a comparable cell, prior to the method or in the absence of the method. In some cases, the method includes measuring the increase transduction of the virus, for example by determining the level of the virus in the cell. Methods of measuring transduction efficiency are known in the art and include, but are not limited to Fluorescence imaging, in vitro and in vivo luminometry, immunohistochemistry, PCR, ddPCR and flow cytometry.

### Methods and Uses of Increasing Virally-Encoded Transgene Expression

The present application also includes a method, optionally an in vitro method, of increasing virally-encoded transgene expression comprising administering a compound of the application and the virus to a cell. Also included is a use of a compound of the application for increasing virally-encoded transgene expression, as well as a compound of the application for use in increasing virally-encoded transgene expression. Optionally, the virally-encoded transgene is a therapeutic gene.

In some embodiments, expression of the transgene is increased 1.1 fold or more, 1.2 fold or more, 1.5 fold or more, 2 fold or more, 2.5 fold or more, 3 fold or more, 5 fold or more, or 10 fold or more, e.g., compared to expression of the transgene, prior to the method or in the absence of the method. In some cases, the method includes measuring the expression of the transgene. Measuring expression levels of a transgene can be done by any method known in the art, including but not limited to measuring levels of nucleic acid expression or expression levels of protein encoded by the transgene.

Methods and Uses of Increasing Virus Growth and/or Virus Spread

The present application also includes a method, optionally an in vitro method, of increasing virus growth and/or virus spread in cells comprising administering a compound of the application to the cells prior to, after or concurrently with the virus. Also included is a use of a compound of the application for increasing virus growth and/or virus spread, as well as a compound of the application for use in increasing virally-encoded transgene expression for increasing virus growth and/or virus spread.

In some embodiments, virus growth and/or virus spread is increased 1.1 fold or more, 1.2 fold or more, 1.5 fold or more, 2 fold or more, 2.5 fold or more, 3 fold or more, 5 fold or more, or 10 fold or more, e.g., compared to virus growth and/or virus spread, prior to the method or in the absence of the method. In some cases, the method includes measuring virus growth and/or virus spread.

To be clear, in the above methods and uses, the term “a compound of the application” also includes embodiments wherein one or more compounds of the application are referenced or formulated in a composition as described herein.

### IV. Methods of Preparing the Compounds and Compositions of the Application

Compounds of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources or may be extracted from cells, plants, animals or fungi. Other starting materials, for example as described below, are readily prepared from available precursors using straightforward transformations that are well known in the art.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

Throughout the processes, it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

For example, methods such as those described in U.S. Pat. No. 7,951,810 may be used.

### EXAMPLES

The following non-limiting examples are illustrative of the present application.

### General Methods

Pevonedistat (MLN4924), the primary drug of this study, was obtained from Cedarlane Labs (cat. A11260) dissolved in dimethyl sulfoxide (DMSO). TAS4464 (HY-128586) and ZM223 (HY-101790) were obtained from MedChemExpress. The remainder of drugs, chemical and cytokines along with their supplies, catalog numbers and solvent are listed in Table 2.

TABLE 2
List of drugs, chemicals and cytokines

| Name | Solvent | Supplier | Catalog no. |
|---|---|---|---|
| Pevonedistat | DMSO | Cedarlane Labs | A11260 |
| (MLN4924) | | | |
| TAS4464 | DMSO | MedChem Express | HY-128586 |
| ZM223 | DMSO | MedChem Express | HY-101790 |
| Human TNF-alpha | PBS | Cedarlane Labs | 210-TA-020 |
| Human IFN-beta | PBS | PBL Assay | 11415-1 |
| | | Science | |
| Human IFN-alpha | PBS | PBL Assay | 11200-1 |
| | | Science | |
| Poly I:C | PBS | Invivogen | tlrl-pic |
| Z-VAD-FMK | DMSO | Promega | G7231 |
| Human TNF-alpha | PBS | R&D Systems | MAB210-SP |
| neutralizing | | | |
| antibody | | | |

DMSO = Dimethyl Sulfoxide,
PBS = Phosphate-Buffered Saline

### Cell Lines

Cell lines used along with their species, tissue type, supplier and catalog number are outlined in Table 3. Cells either utilized Dulbecco's modified Eagle's medium (DMEM; HyClone™ cat. 10-013) or RPMI 1640 medium (Corning) supplemented with 1% (v/v) penicillin-streptomycin (Gibco), 30 mM HEPES buffer, and 10% (v/v) Fetal Bovine Serum (Gibco, cat. 12483020) or 10% (v/v) serum composed of 3-parts HyClone newborn calf serum (Thermo Fisher, cat. SH3011803) and 1-part Fetal Bovine Serum (Gibco, cat. 12483020). Cell lines were maintained in 37° C. and 5% CO₂ conditions in a humidified incubator. Phase and fluorescence images were taken using the EVOS Live Cell Imaging System (Thermo Fisher).

TABLE 3
List of cell lines

| Name | Species | Tissue and type | Supplier | Catalog no. |
|---|---|---|---|---|
| 786-0 | Human | Renal cell | ATCC | CRL-1932 |
| | | adenocarcinoma | | |
| Vero | African Green | Renal epithelial | ATCC | CCL-81 |
| | Monkey | | | |
| 76-9 | Human | Rhabdomyosarcoma | **** | |
| A549 | Human | Lung carcinoma | ATCC | CCL-185 |
| MCF7 | Human | Breast | ATCC | HTB-22 |
| | | adenocarcinoma | | |
| HeLa | Human | Cervical | ATCC | CCL2 |
| | | adenocarcinoma | | |
| HT1080 | Human | Fibrosarcoma | * | |
| THP-1 | Human | Acute Monocytic | ** | |
| | | Leukemia | | |
| 4T1 | Mouse | Breast carcinoma | ATCC | CRL-2539 |
| B16F10 | Mouse | Melanoma | ATCC | CRL-6475 |
| CT26WT | Mouse | Colon carcinoma | ATCC | CRL-2638 |
| CT2A | Mouse | Glioma | * | |
| DBT | Mouse | Astrocytoma | * | |
| ID8 | Mouse | Ovarian | *** | |
| L1210 | Mouse | Leukemia | ** | |
| PAN02 | Mouse | Pancreatic | ATCC | PTA-10395 |
| S-180 | Mouse | Sarcoma | ATCC | CCL-8 |
| D17 | Canine | Osteosarcoma | ATCC | CCL-183 |

ATCC = American Type Culture Collection.
* denotes generously gifted by Dr. John Bell of the Ottawa Hospital Research Institute (OHRI, Ottawa, Canada),
** denotes generously gifted by Dr. William Stanford of the OHRI,
*** denotes generously gifted by Dr. Barbara Vanderhyden of the OHRI,
**** denotes generously gifted by Drs. Korneluk and Holick of the Children's Hospital of Eastern Ontario (CHEO, Ottawa, Canada).

Primary murine hepatocytes were generously supplied by Dr. Morgan Fullerton and Conor O'Dwyer (University of Ottawa) and were isolated as previously described (1). Primary ovarian cancer patient-derived cell lines (OVAPT) were derived from patient ascites fluid were obtained from routine paracentesis according to Ottawa Health Science Network Research Ethics Board (OHSN-REB) protocol #20140075-01H. Primary human glioblastoma (PriGO) cells were established from surgically resected tumors from patients at The Ottawa Hospital and were obtained as a generous gift from Dr. Ian Lormier of the Ottawa Hospital Research Institute (Ottawa, Canada). PriGO cells were grown on laminin-coated plates using serum-free Neurobasal A (NA) media supplemented with epidermal growth factor (EGF), fibroblast growth factor 2 (FGF2), B-27 and N-2, and maintained in 37° C., 5% O2 and 20% CO₂ conditions in a humidified incubator.

### Oncolytic Viruses

Rhabdovirus: Indiana serotype of VSV wild-type (VSV-WT) or harboring a deletion of methionine 51 in the M protein (VSVΔ51) and insertion of green fluorescence protein (GFP) or firefly luciferase (FLuc) were used throughout the Examples. All viruses were propagated on Vero cells and purified on 5-50% OptiPrep™ (Sigma-Aldrich, St. Louis, MO) gradients. Viral titers were determined by standard plaque assay on Vero cells according to published protocol (2) or by high throughput titration as previously described (6)

Herpes Simplex Virus: HSV-1 N212 expressing GFP was obtained as a generous gift from Dr. Karen Mossman of McMaster University (Hamilton, Canada). Viral titers were determined by standard plaque assay on Vero cells according to published protocol (3).

Vaccinia Virus: The VV Wyeth strain harboring a disruption of thymidine kinase (TK) and vaccinia growth factors genes, and insertion of GFP (VVdd) was obtained as a generous gift from Dr. Andrea McCart of Mount Sinai Hospital (Toronto, Canada). Viral titers were determined by standard plaque assay on U2OS cells according to published protocol (4).

Maraba Virus: The Maraba MG1 virus, tagged with firefly luciferase or eGFP, was harvested, and purified as previously described (5). Viral titers were determined by standard plaque assay or high throughput titration on Vero cells (6).

Adenovirus: Adenovirus, serotype 5 (Ad5) tagged with firefly luciferase (FLuc) was obtained as a gift by J. Gauldie (McMaster University).

### High Throughput Viral Titration

Vero cells were seeded at a density of 2.5×10⁴ cells/well in opaque white bottom 96-well microplates (Thermo Fisher, cat. 07-200-628). 20 μL of sample supernatant was transferred to the microplates and incubated for 5-7 hours. The automated addition of 25 μL of luciferin solution (2 mg/mL constituted in sterile PBS, Cedarlane Labs, cat. 122799(PE)) was performed, and mean luminescence read after 30 seconds. Readings were analyzed in comparison to a standard curve. Refer to published protocol for further details (6).

### In Vivo Mouse Tumor Models

CT26WT: 6-week-old BALB/c mice (Charles River Laboratories) were subcutaneously implanted with a bolus of 100 μL PBS containing 3×10⁵ syngeneic CT26WT colon carcinoma cells in the right flank. After 11 days when tumor volumes reach roughly 100 mm³, tumors were injected intratumorally with pevonedistat (90 mg/kg) or vehicle alone. Four hours later, tumors were injected intratumorally with a bolus of 25 μL PBS containing 1×10⁸ pfu of VSVΔ51. This treatment regimen was repeated two more times, spaced one day apart. In rechallenge experiments, mice in remission were injected with 5×10⁵ CT26WT cells in the opposite (left) flank, then monitored for tumor volume and survival.

4T1: 6-week-old BALB/c mice (Charles River Laboratories) were subcutaneously implanted with a bolus of 100 μL PBS containing 5×10⁵ 4T1 syngeneic 4T1 mammary carcinoma cells in the right flank. After 9 days when tumor volumes reach roughly 100 mm³, tumors were injected intratumorally with pevonedistat (90 mg/kg) or vehicle alone. Four hours later, tumors were injected intratumorally with a bolus of 25 μL PBS containing 1×10⁸ pfu of VSVΔ51. This treatment regimen was repeated two more times, spaced one day apart. For survival studies, mice were end pointed when tumor volumes reached greater than 1500 mm³ or showed significant respiratory distress from lung metastases. Mice were randomized to different treatment groups according to tumor size prior to the first treatment. All experiments were performed in accordance with the University of Ottawa Animal Care and Veterinary Service guidelines for animal care, under the protocols OHRI-2264 and OHRI-2265.

### Human and Murine Ex Vivo Tumor Models

BALB/c mice were subcutaneously implanted with 3×10⁵ CT26WT colon carcinoma cells or C57BL/6 mice were subcutaneously implanted with 3×10⁵ 76-9 rhabdomyosarcoma cells. Upon reaching a tumor volume of 1500 mm³, mice were culled, and tissues of interest were extracted. For human tissue samples, tumor samples were obtained from patients undergoing surgical resection who provided informed consent in accordance with Declaration of Helsinki guidelines. The global tissue collection program was approved by the OHSN-REB under the protocol numbers OHSN-REB #2003109-01H and OHSN-REB #20120559-01. Tissues were processed into 2 mm slices and 2 mm diameter circular cores were taken using a punch biopsy tool. Cores were maintained in humidified incubators at 37° C., 5% CO₂ in DMEM supplemented with 10% serum, 30 mM HEPES, 1% (v/v) penicillin-streptomycin and 0.25 mg/L amphotericin B. Cores were treated with pevonedistat 4 hours, then infected with VSVΔ51-GFP at 3×10⁴ pfu/core. After 48 hpi, fluorescence images were taken using the EVOS Live Cell Imaging System (Thermo Fisher) and the supernatant was analyzed for viral titer by standard plaque assay as described previously.

### Quantitative Real-Time Polymerase Chain Reaction

RNA from lysed cells were homogenized using the QIAshredder (Qiagen, cat. 79656), then extracted from lysed cells using the QIAGEN RNeasy kit (Qiagen, cat. 74106) according to manufacturer's protocol and quantified using a NanoDrop™ One Microvolume UV-Vis Spectrophotometer (Thermo Fisher Scientific, Rockford, IL). To generate cDNA, the RevertAid H-Minus First Strand cDNA Synthesis Kit (Thermo Fisher, cat. K1632) was used. Resulting nucleic acid was subject to quantitative real-time PCR using primers outlined in Table 4, Applied Biosystems PowerUp SYBR Green Master Mix (Thermo Fisher, cat. A25776) in a 7500 Fast Real-Time PCR system (Applied Biosystems, Foster City, CA). Gene expression was calculated using the Pfaffl method.

TABLE 4
List of primers

| Model | Gene | Forward primer (5′ to 3′) | Reverse primer (5′ to 3′) |
|---|---|---|---|
| VSV | M | ATACTCAGATGTGGCAGCCG | GATCTGCCAATACCGCTGGA |
| | N | GATAGTACCGGAGGATTGACG | TCAAACCATCCGAGCCATTC |
| Human | STAT1 | ATGGCAGTCTGGCGGCTGAAT | CCAAACCAGGCTGGCACAATT |
| | | T | G |
| | STAT2 | CAGGTCACAGAGTTGCTACAG | CGGTGAACTTGCTGCCAGTCT |
| | | C | T |
| | GAPDH | ACAGTCAGCCGCATCTTCTT | GTTAAAAGCAGCCCTGGTGA |
| | IFN-β | CATTACCTGAAGGCCAAGGA | CAGCATCTGCTGGTTGAAGA |
| | IL-1β | CCACAGACCTTCCAGGAGAAT | GTGCAGTTCAGTGATCGTACA |
| | | G | GG |
| | TNF-α | GCTGCACTTTGGAGTGATCG | GAGGGTTTGCTACAACATGGG |
| | CCL5 | GCAGTCGTCCACAGGTCAAG | TCTTCTCTGGGTTGGCACAC |
| | IL-6 | ACCCCCAATAAATATAGGACTG | GAAGGCGCTTGTGGAGAAGG |
| | | GA | |
| | IFITM1 | CCGTGAAGTCTAGGGACAGG | GGTAGACTGTCACAGAGCCG |
| | IRF7 | GCAAGGTGTACTGGGAGCG | GATGGTATAGCGTGGGGAGC |
| | IRF9 | TTCTTCAAGGCCTGGGCAAT | CCTGGTGGCAGCAACTGATA |
| | NEDD8 | CGCTGACCGGAAAGGAGATT | CAGAGCCAACACCAGGTGAA |

### Cell Viability Assay

Resazurin metabolic dye (Millipore Sigma, cat. S103200) was added to samples at a 1:10 dilution and incubated for 2 hours. Using a BioTek Microplate Reader (Norgen BioTek Corp, Ontario, Canada) and Gen5 2.07 software, fluorescence was measured at 590 nm upon excitation at 530 nm. Readings were expressed relative to the average of the uninfected, mock treated condition.

### Caspase-8 Luminescent Assay

786-0 seeded in a 96-well white plate were subject to treatment with pevonedistat or vehicle, then infection by VSVΔ51 or vehicle four hours later. At the specified time-point of interest, cells were assayed using the Caspase-Glo® 8 Assay System (Promega, cat. G8201) according to manufacturer's instructions. Luminescence readings were taken using the BioTek Microplate Reader (Norgen BioTek Corp, Ontario, Canada).

### Enzyme-Linked Immunosorbent Assay (ELISA)

Supernatant from treated and infected 786-0 cells seeded in a 24-well plate were collected 24 hpi and quantified for human IFN-β concentration using the Human IFN Beta ELISA Kit (PBL Assay Science, cat. 41410) according to manufacturer's protocol. Absorbance readings were taken using the BioTek Microplate Reader (Norgen BioTek Corp, Ontario, Canada) at 450 nm.

### RNA-Sequencing Analysis

Two biological replicates of RNA were extracted from lysates of treated cells and quantified as described above. Pooled samples were then shipped to the Donnelly Sequencing Centre (University of Toronto) and mRNA-seq libraries were generated using the NEB NEBNext™ Ultra II Directional RNA library prep kit according to manufacturer's protocol. Libraries were sequenced using the Illumina NextSeq™500 with single-end 75 bp reads. After sequencing, resulting fastq files were checked for quality using FastQC (Babraham Bioinformatics, United Kingdom). Pseudo alignment and transcript quantification were performed with KALLISTO (7), and differential expression was determined using SLEUTH (8). Gene ontology analysis was performed using the Gene Ontology enrichment analysis and visualization tool (GOrilla) (9). Identification of involved transcription factors was performed using the TFactS tool (de Duve Institute, Université Catholique de Louvain, Brussels, Belgium) (10).

### Immunoblotting

Sample were washed twice with cold PBS and lysed for 10 minutes at 4° C. using 50 mM HEPES, 150 mM NaCl, 10 mM EDTA, 10 mM Na₄P₂O₇, 100 mM NaF, 2 mM Na₃VO₃, protease inhibitor cocktail (Roche), phosphatase inhibitor cocktail (Cell Signaling Technology, cat. 5870S) and 1% Triton™ X-100. Resulting cell lysates were centrifuged to remove cellular debris. For nuclear and cytoplasmic fractionation, the NE-PER Nuclear and Cytoplasmic Extraction Kit (Thermo Fisher Scientific) was used according to manufacturer's protocol. Protein concentrations were quantified using the Pierce BCA Protein Assay Kit (Thermo Fisher, cat. 23225). 20 μg was loaded with 4× NuPAGE™ LDS Sample Buffer (Thermo Fisher, cat. NP0007) into 4-15% Mini-PROTEAN™ Gels (Bio-Rad, Mississauga, ON), electrophoresed using the Mini Trans-Blot™ Cell system (Bio-Rad, Mississauga, ON) and transferred onto nitrocellulose membrane using the Trans-Blot Turbo RTA Mini Transfer Kit according to manufacturer's protocol (Bio-Rad, cat. 1704270). Blots were blocked with 5% BSA for 1 hours, then probed with respective primary and secondary antibodies. Bands were visualized using Clarity Western ECL Substrate (Bio-Rad, cat. 1705061) on a ChemiDoc Touch Imaging System (Bio-Rad, Mississauga, ON).

### Immunocytochemistry

Cells were seeded on 12 mm glass round coverslips (Thomas Scientific, cat. 64-0712) in a 12-well format. Following indicated experimental processes, cells were washed twice with PBS* (PBS supplemented with 1 mM CaCl₂, 500 μM MgCl₂), fixed using 4% paraformaldehyde (PFA) for 30 minutes, permeabilized using a 0.2% Triton-X 100 in a 200 mM glycine/PBS* solution for 7 minutes, then quenched in 200 mM glycine/PBS* for 15 minutes. Slides were then blocked using 5% BSA/PBS* for 1 hour at room temperature, then incubated overnight with the respective primary antibody in humidified chamber at 4° C. After two washes with PBS*, corresponding secondary antibodies were applied for 1 hour, then samples were mounted onto glass slides and counterstained using Prolong gold anti-fade with 4′,6-diamidino-2-phenyl-indole (Molecular Probes). Slides were imaged using a Zeiss Axiocam™ HRM Inverted fluorescent microscope (Zeiss, Toronto, Canada) and images were processed using Axiovision™ 4.0 software. Quantification of fluorescent intensities were performed using CellProfiler 3.0.0 (Massachusetts Institute of Technology, Cambridge, USA).

### Flow Cytometry

786-0 cells were treated and infected with VSVΔ51-GFP in a 6-well format. At 24 hpi, cells were collected, washed, and stained with propidium iodide (PI) (Biolegend, cat. 421301) or Annexin V (Cedarlane labs, cat. 640934) according to manufacturer's protocol. Samples were then analyzed for PI staining and GFP signal by flow cytometry on a BD LSRFortessa™. Acquired data was analyzed using FlowJo™ software.

Small-Interfering RNA (siRNA)

786-0 cells were seeded at 40% density in 24-well plates in serum-free DMEM overnight. Cells were then transfected either with control, scramble RNA (ON-TARGETplus Non-targeting Control Pool, #D-001810-10-05, Horizon Discovery) or a SMARTpool of siRNA targeting NEDD8 (ON-TARGETplus NEDD8 siRNA, #L-020081-00-0005, Horizon Discovery) using the Lipofectamine™ RNAiMAX Transfection Reagent (Thermo Fisher, cat. 13778075) according to manufacturer's protocol in Opti-MEM™ I Reduced Serum Medium (Thermo Fisher, cat. 31985062). After 6 hours, media containing siRNA was replaced with DMEM supplemented with 1% (v/v) penicillin-streptomycin (Gibco), 30 mM HEPES buffer and 10% (v/v) serum composed of 3-parts HyClone newborn calf serum (Thermo Fisher, cat. SH3011803) and 1-part Fetal Bovine Serum (Gibco, cat. 12483020). Upon reaching approximately 80% confluency, cells were treated with reagents or infected with VSVΔ51 as specified.

### Statistics

Statistical analyses were performed using Prism 9 (GraphPad, San Diego, CA) software. Viral titer and mRNA expression values were log-transformed prior to analysis. Viability measures were normalized to untreated, uninfected cells as indicated in figure legends. Statistical tests were performed as indicated by figure legends including Student's t-test, one-way analysis of variance (ANOVA) with Tukey's multiple comparisons test, and two-way ANOVA. Two-tailed testing was used unless otherwise specified. Kaplan-Meier curves were graphed for survival studies and differences detected using the log-rank test. Error bars represent the standard error from the mean (SEM). A P-value less than 0.05 was considered statistically significant.

### Example 1—Pevonedistat as Viral Sensitizer on Cancer Cells

### Pevonedistat Sensitizes Cancer Cells to Oncolytic VSVΔ51 Infectivity

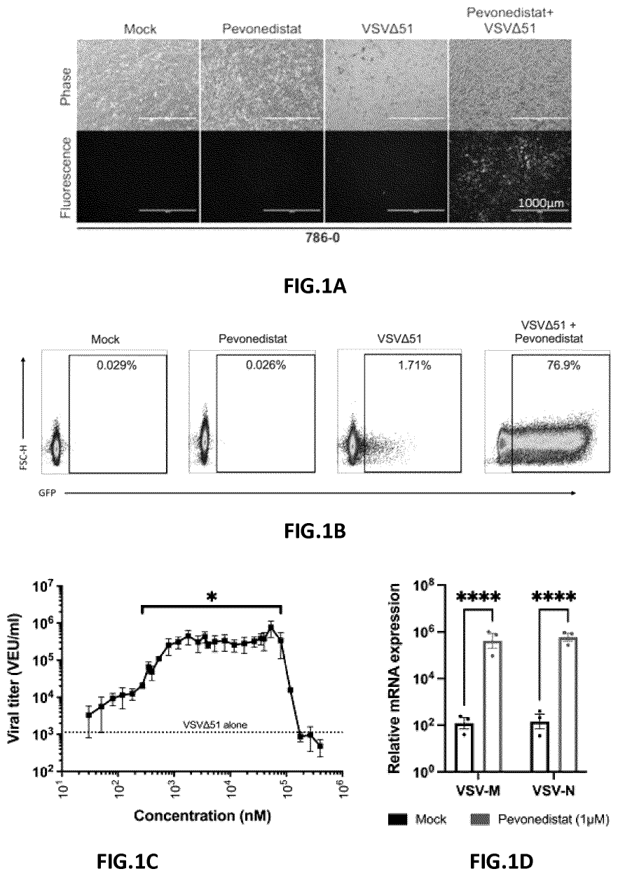

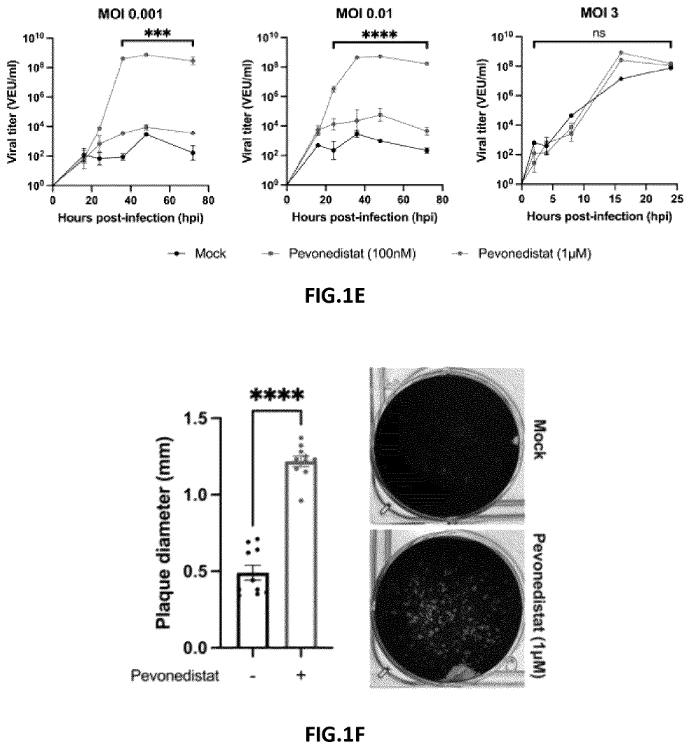

To characterize the viral sensitizing properties of pevonedistat, human renal 786-0 carcinoma cells, a model naturally resistant to VSVΔ51 infection, were first pre-treated with a standard dose of 1 μM for 4 hours, then infected with VSVΔ51 tagged with green fluorescent protein (VSVΔ51-GFP) at a low multiplicity of infection (MOI). At 24 hours post infection (hpi), a marked increase in viral GFP transgene expression was demonstrated by fluorescent microscopy (FIG. 1A) and flow cytometry (FIG. 1B), supporting the robust enhancement of VSVΔ51 infectivity by pevonedistat. Further investigation by high-throughput titration using a wide concentration range of pevonedistat revealed that pevonedistat was able to significantly increase viral titer compared to VSVΔ51-infected only cells approximately across a range of 180 nM to 120 μM (FIG. 1C). Moreover, it was demonstrated that administration of pevonedistat as early as 24 hours prior until 4 hours after infection was capable of increasing VSVΔ51 viral titer highlighting its rapid uptake and extended pro-viral mechanism of action. To confirm increased levels of VSVΔ51, RNA extracted from these cells was analyzed for VSV genome expression by quantitative polymerase chain reaction. Expectedly, messenger RNA (mRNA) levels of VSV matrix protein (M) and nuclear protein (N) were significantly increased in pevonedistat-treated cells compared to their untreated counterparts (FIG. 1D).

Further investigation into the ability of pevonedistat to potentiate viral infectivity was explored by comparing multi-step to single-step growth curves. Pevonedistat was able to robustly enhance VSVΔ51 when infected at a low MOI of 0.001 or 0.01, but not at a high MOI of 3 by high-throughput titration (FIG. 1E). While not wishing to be bound by theory, this suggests that pevonedistat promotes viral spread to increase its growth, but not through increasing the rate of VSVΔ51 replication or viral entry. Indeed, analysis of viral spread by plaque expansion assay demonstrated that pevonedistat significantly increased the average plaque diameter of each viral foci in a monolayer of 786-0 cells as visualized by Coomassie blue stain (FIG. 1F).

### Pevonedistat Confers VSVΔ51 Viral Sensitization Across a Variety of Tumor Models

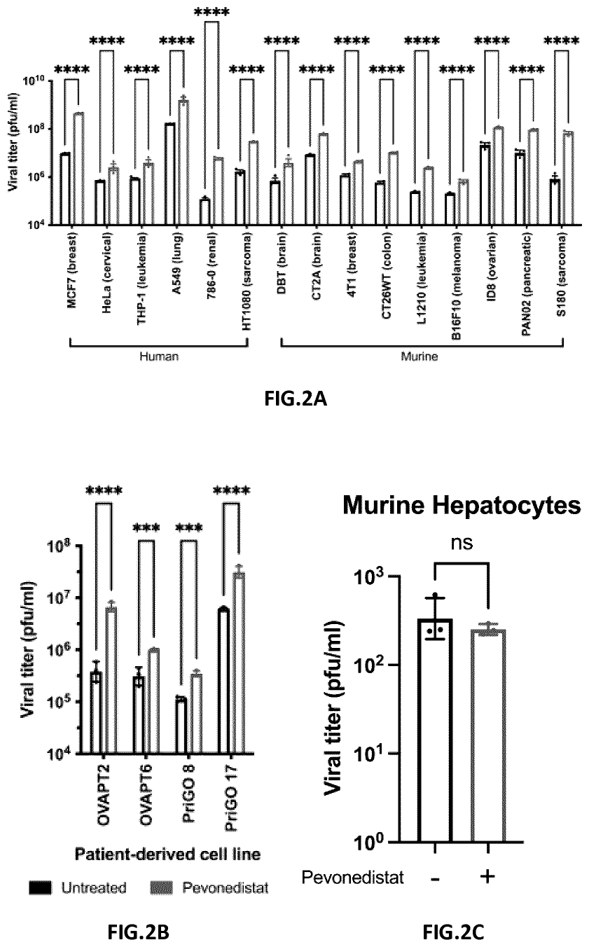

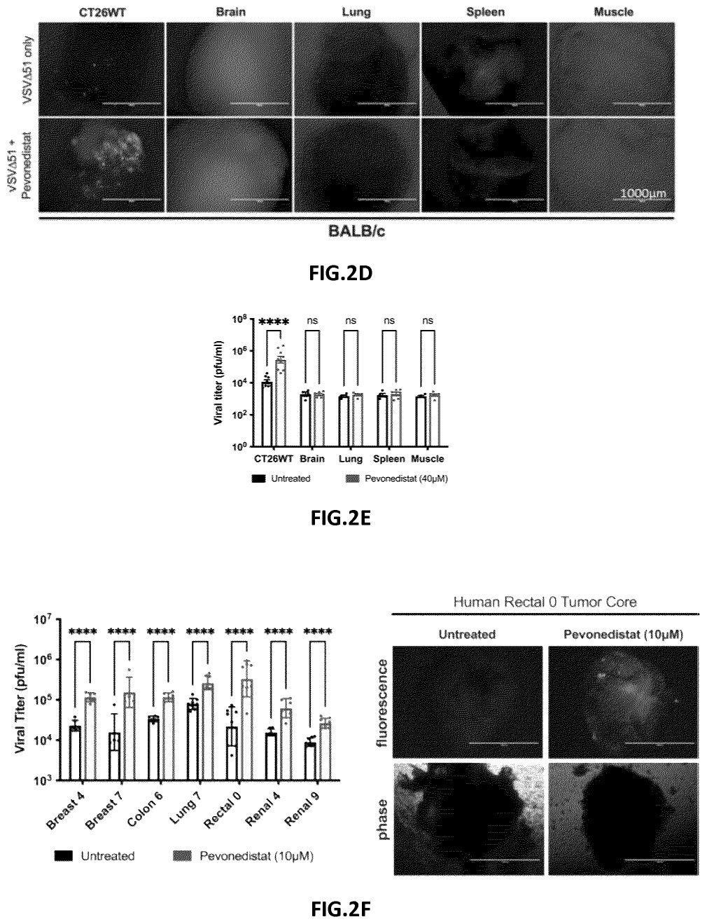

Given that pevonedistat is currently under clinical investigation for its antitumor effect in different solid and hematological cancers, it was sought to establish its viral sensitizing ability across a large variety of cancer types. In both human and murine models, it was successfully demonstrated that pevonedistat increases VSVΔ51-GFP viral titer across different solid and hematological cancer cell lines (FIG. 2A). Fluorescent microscopy confirms increased VSVΔ51-GFP transgene expression in these cell lines. This trend was also observed in human ovarian (OVAPT) and glioma patient-derived cell lines (PriGO) where pevonedistat also significantly increased VSVΔ51 viral titer as determined by plaque assay (FIG. 2B). Moreover, pevonedistat does not increase viral replication in isolated primary murine hepatocytes, further demonstrating that tumor selectivity is maintained (FIG. 2C).

To test this phenomenon in an ex vivo model, murine CT26WT colon carcinoma cells were implanted into BALB/c mice. Mice were culled upon reaching a tumor volume of 1500 mm³. Normal brain, lung, spleen and muscle cores, and tumor cores were extracted, pre-treated with pevonedistat (40 μM) for 4 hours, then infected with VSVΔ51-GFP. Fluorescent images 24 hpi confirmed that pevonedistat has significant viral sensitizing properties in tumors ex vivo (FIG. 2D, FIG. 2E). In fact, pevonedistat was able to increase VSVΔ51 viral titer by over 30-fold in the CT26WT model as indicated by viral plaque assay. Similar results were also obtained in ex vivo 76-9 rhabdomyosarcoma cores in C57BL/6 mice. When tested in primary human ex vivo clinical samples, pevonedistat was also able to increase VSVΔ51 infection across a large variety of tumor types including breast, colon, lung, rectal and renal as demonstrated by viral plaque assay and fluorescent microscopy (FIG. 2F). In the rectal cancer sample, pevonedistat increased the viral titer by over 15-fold. Together, these results demonstrate that pevonedistat's ability to increase VSVΔ51 viral infectivity is applicable across a broad range of tumor contexts.

### Pevonedistat Increases VSVΔ51-Mediated Oncolysis Through Apoptotic Pathways

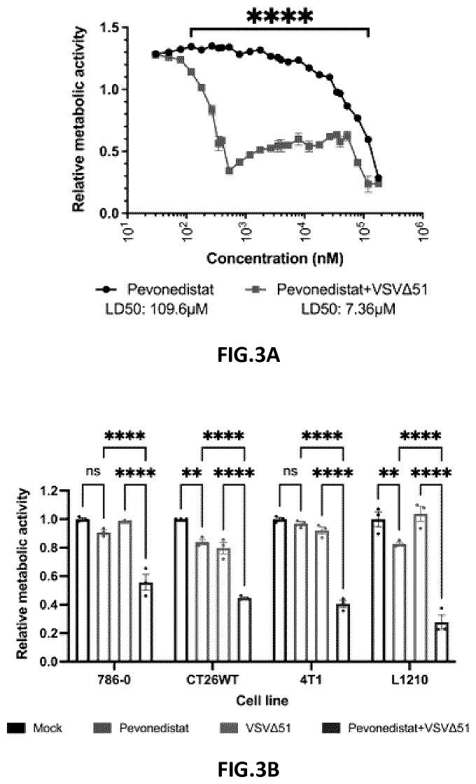

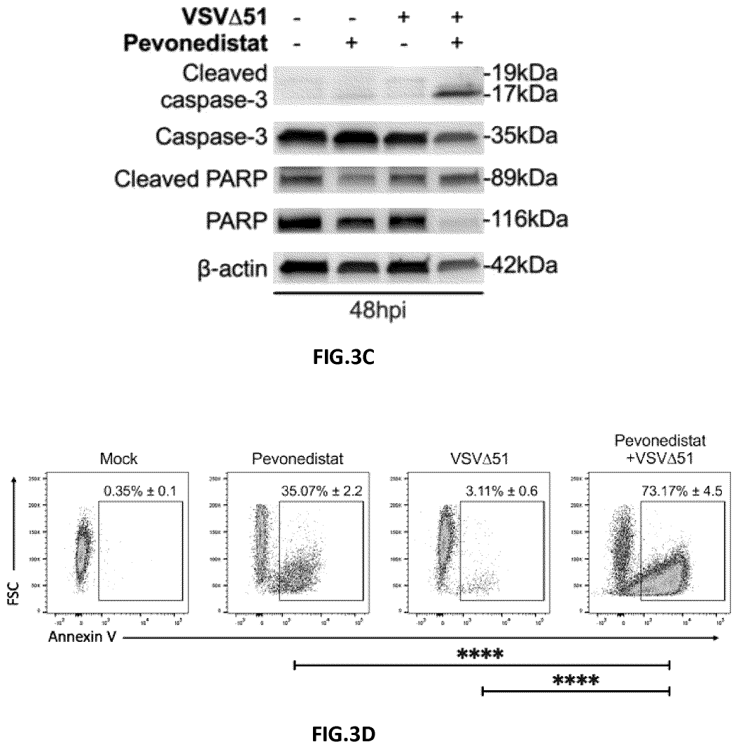

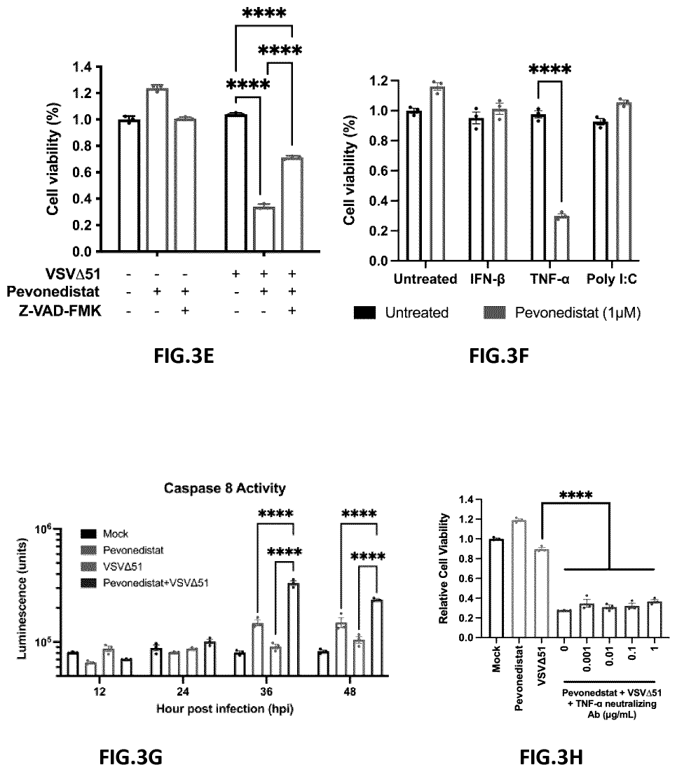

Similar to other small molecules with viral sensitizing properties, it was hypothesized that pevonedistat could enhance the apoptosis-mediated oncolysis of VSVΔ51. 786-0 cells were pre-treated with various concentrations of pevonedistat for 4 hours and infected with VSVΔ51 (MOI 0.01). Cell viability was then assayed using resazurin metabolic dye 48 hpi. While pevonedistat on its own had a calculated median lethal dose (LD₅₀) of 109.6 μM, the addition of VSVΔ51 robustly reduced the LD₅₀ to 7.36 μM with significant differences detected at a concentration as low as 120 nM (FIG. 3A). This increased cytotoxicity was also observed across several other solid and hematological tumor types (FIG. 3B). Conversely, isolated primary murine hepatocytes treated with pevonedistat and infected with VSVΔ51 showed no significant viability differences from hepatocytes treated only with pevonedistat.

Next, to establish the role of apoptosis, 786-0 cells treated in combination with pevonedistat and VSVΔ51 (MOI 0.1) were lysed 48 hpi and probed for downstream effectors of apoptosis by western blot (FIG. 3C). As expected, cells treated with the combination showed markedly higher levels of cleaved caspase-3 and decreased levels of full-length poly (ADP-ribose) polymerase (PARP), indicating their increased activation. Similarly, flow cytometry analysis of these cells following Annexin V staining demonstrated a significantly greater proportion of Annexin V-positive cells in the combination treatment compared to either monotherapy, suggesting increased apoptosis in this population (FIG. 3D). Moreover, the addition of the broad-spectrum caspase inhibitor Z-VAD-FMK was able to significantly rescue cell viability in cells co-treated with pevonedistat and VSVΔ51 from 33% to 71%. Without wishing to be bound to theory, these results appear to confirm that the oncolytic impact of pevonedistat is attributable to caspase-dependent apoptotic mechanisms (FIG. 3E).

The attenuated oncolytic VSVΔ51 primarily induces cell killing through the death receptor apoptotic pathway (11). The role of the death-induced signaling complex (DISC) in the present mechanism was investigated. Indeed, it was found that pevonedistat combined with external TNF-α stimulation was able to induce increased cell killing and apoptosis activation, unlike other commonly secreted cytokines in response to VSVΔ51 infection such as IFN-β or poly I:C (FIG. 3F). It was then sought to assess the activity of caspase 8, the subsequent initiator to the extrinsic apoptotic cascade, which was analyzed via luciferase assay. The present data revealed that caspase-8 activity in cells treated in combination with pevonedistat and VSVΔ51 peaked at 36 hpi and remained significantly increased 48 hpi compared to all other conditions (FIG. 3G). Without wishing to be bound to theory, the addition of neutralizing TNF-α antibodies was unable to negate increases in cell toxicity by pevonedistat suggesting that this effect is likely not caused by an increase TNF-α secretion and receptor activation (FIG. 3H). These data support that pevonedistat lowers the threshold needed to induce cell death apoptotic mechanisms by VSVΔ51 infection.

### Pevonedistat Improves Oncolytic VSVΔ51 Therapeutic Efficacy In Vivo

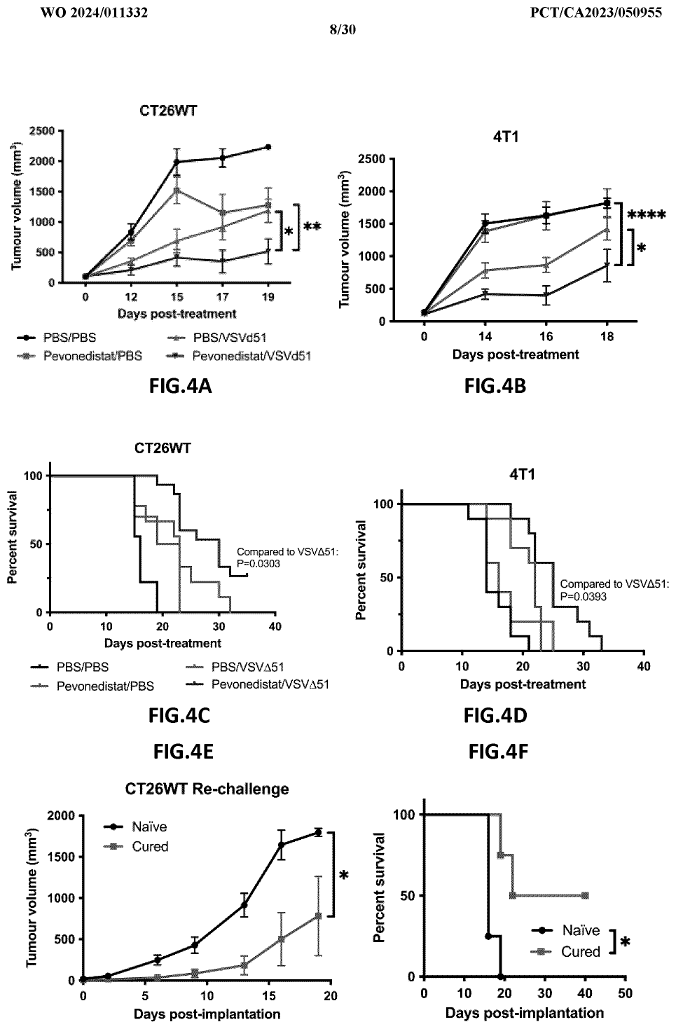

Upon establishing the potentiation of VSVΔ51 oncolytic efficacy by pevonedistat, it was investigated whether this combinational treatment regimen would improve the anti-cancer therapeutic efficacy of oncolytic VSVΔ51 therapy in mouse models of cancer. Syngeneic murine colon CT26WT or mammary 4T1 carcinoma cells, both of which demonstrated marked viral sensitization responses in vitro, were subcutaneously implanted into 6-week-old BALB/c mice and allowed to progress to 100 mm³. Mice were then injected intratumorally with pevonedistat (90 mg/kg), then 1×108 pfu of VSVΔ51 4 hours later for a total of three treatments spaced one day apart (day 0, 2 and 4). Mice given the combination therapy were successfully able to suppress tumor progression as tumor volumes taken post-treatment were significantly smaller when compared to either monotherapy (FIG. 4A and FIG. 4B). For survival studies, mice were culled according to animal care guidelines when tumor volumes reached 1500 mm³. In accordance with the greater tumor control observed in mice receiving the combination treatment, 4/15 (27%) mice bearing CT26WT tumors (P=0.03 vs. PBS/VSVΔ51 group) achieved complete remission whereas all other mice receiving placebo or monotherapies succumbed to their tumor burden (FIG. 4C). In the more aggressive 4T1 model, the pevonedistat+VSVΔ51 combination also significantly prolonged survival (P=0.04 vs. PBS/VSVΔ51 group) compared to all other conditions (FIG. 4D). These results demonstrate that pevonedistat in combination with VSVΔ51 therapy confers improved therapeutic benefit when compared to mice receiving placebo or either monotherapy.

One of the benefits of oncolytic virotherapy is the induction of anticancer immunological memory. Therefore, it was investigated whether the same phenomenon could be observed for pevonedistat. All four mice cured of CT26WT tumors were re-challenged with CT26WT cells injected subcutaneously in the opposite flank. When compared to their naïve counterparts implanted in parallel (FIG. 4E and FIG. 4F), the cured mice were able to suppress tumor volume progression more effectively 20 days after implantation and demonstrated improved overall survival (P=0.017). Together, the data supports that pevonedistat may enhance systemic antitumor immunological memory in response to oncolytic VSVΔ51 virotherapy.

### Pevonedistat Impairs the Antiviral Type 1 Interferon Response

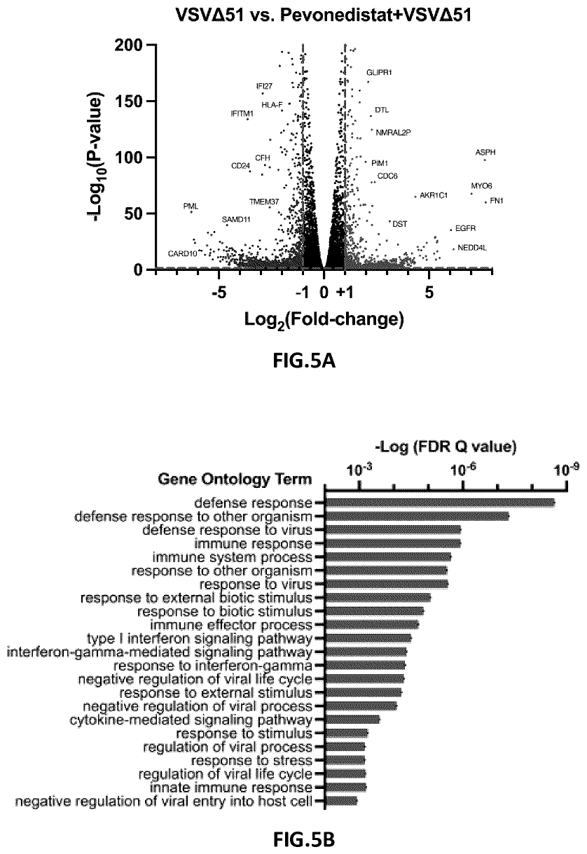

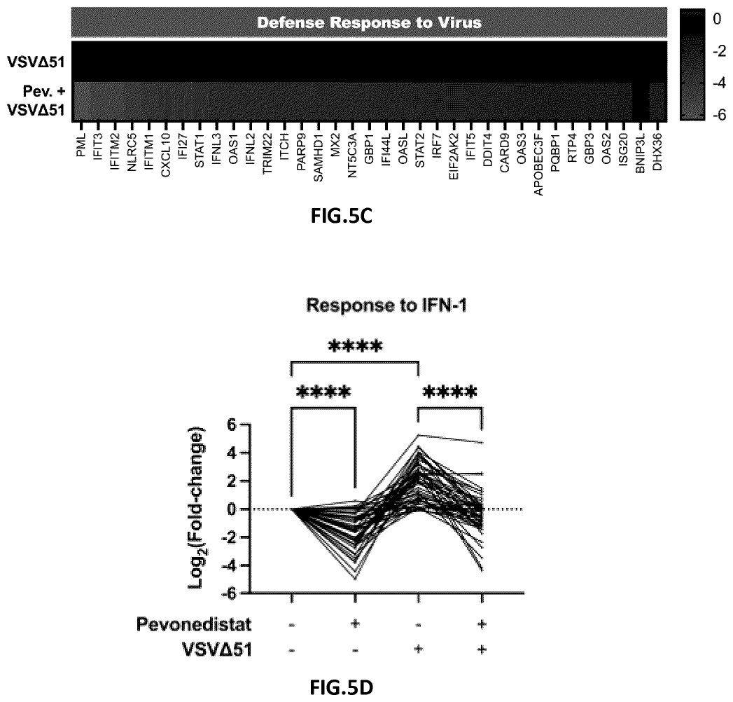

To take an unbiased approach in elucidating the viral sensitizing mechanism of pevonedistat, RNA-sequencing was first employed to analyze whole transcriptome changes in response to pevonedistat and VSVΔ51 combinational therapy. RNA was extracted from 786-0 cells pre-treated with or without pevonedistat (1 μM) for 4 hours and infected with or without VSVΔ51 (MOI 0.01) after 24 hours. After sequencing, gene expression profiles between cells infected with VSVΔ51 treated with or without pevonedistat identified 3,038 genes that were significantly upregulated (P<0.05, log 2-fold change >2) and 3,326 genes that were significantly downregulated (FIG. 5A). Gene ontology (GO) enrichment analyses were performed on ranked lists using the GOrilla tool, which confirmed upregulation of previously established processes of pevonedistat including cellular processes of DNA metabolism, cell cycle regulation and stress responses. More importantly, the GO analysis identified defense responses to virus, immune response and the IFN-1 signaling as being significantly downregulated in both the presence and absence of infection (FIG. 5B). Upon closer inspection of the genes related to the “Defense response to virus” GO term, almost all genes were downregulated upon addition of pevonedistat with the promyelocytic leukemia protein (PML) gene being downregulated by approximately 79-fold (FIG. 5C). Moreover, graphing the log 2-fold change of all genes related to the IFN-1 GO term revealed that these genes were significantly downregulated upon the addition of pevonedistat, even in the absence of VSVΔ51 infection (FIG. 5D). These findings support that pevonedistat behaves similar to many other viral-sensitizing compounds by impairing the IFN-1 response to increase VSVΔ51 sensitivity. However, not only does pevonedistat impair the cellular ability to respond to viral infection but continues to suppress the antiviral response even in the absence of infection.

### Neddylation Inhibition Confers Viral Sensitizing Activity Through STAT1 Inhibition

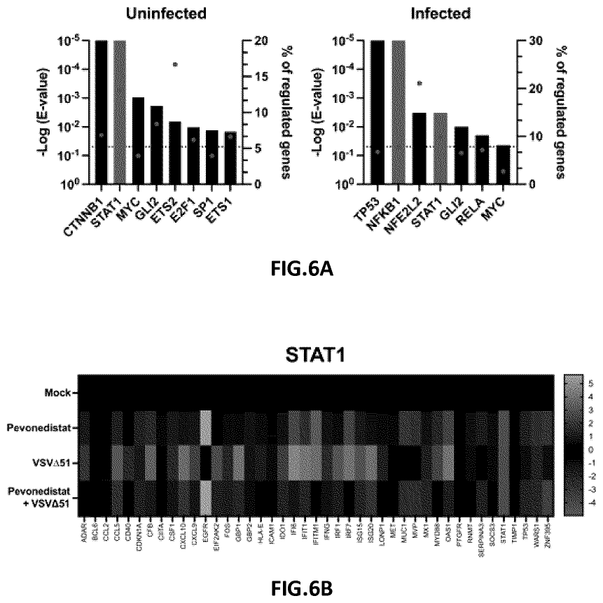

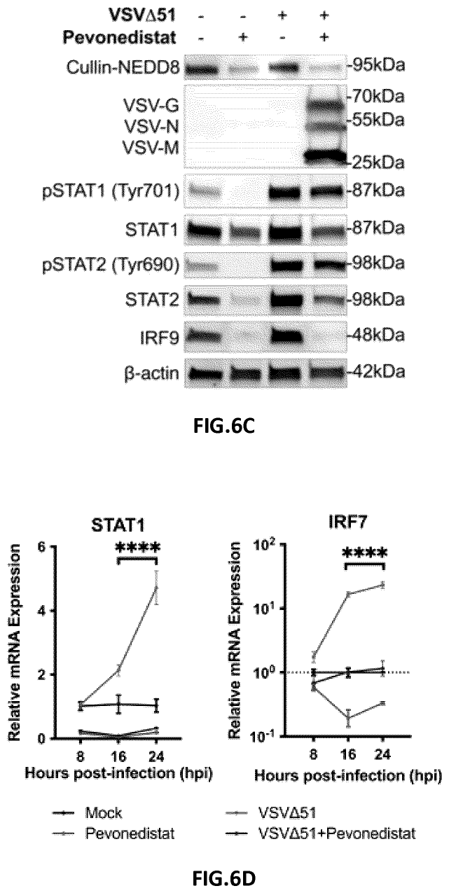

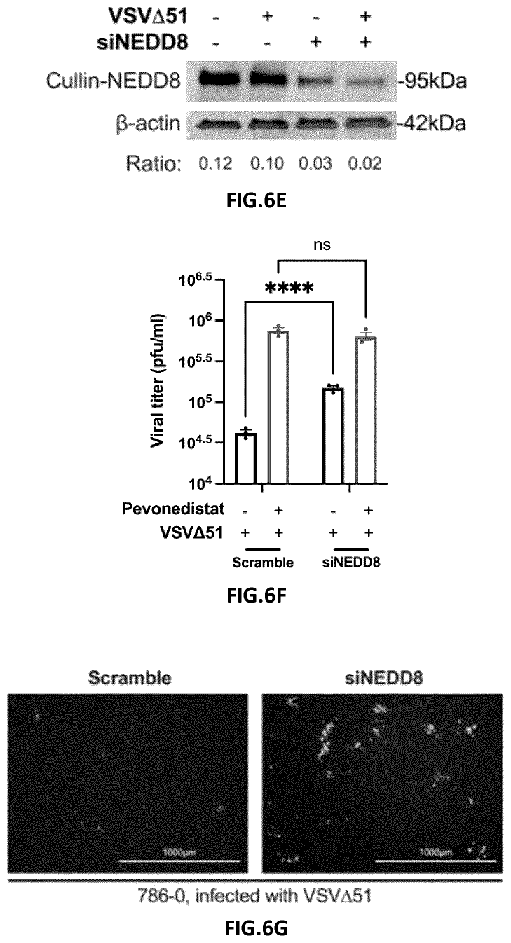

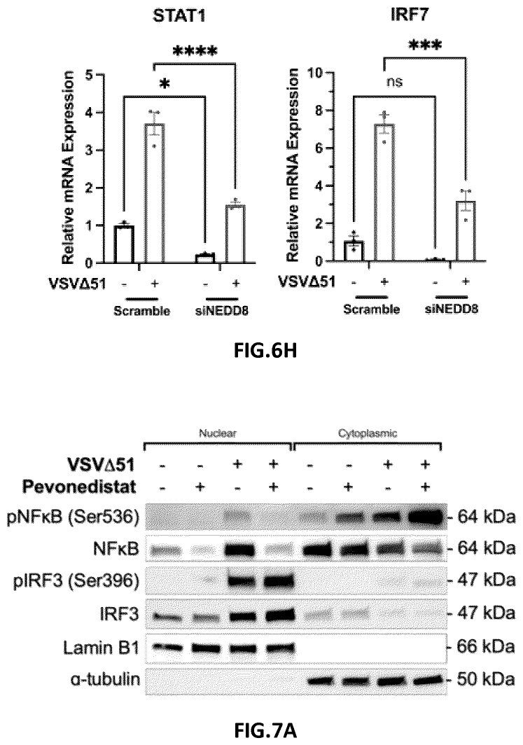

To gain insight into which transcription factors were modulated in response to pevonedistat during VSVΔ51 infection, the RNA sequencing dataset was analyzed by inputting significantly downregulated genes upon addition of pevonedistat using the published TFactS tool (10). The in silico results identified that STAT1 is inhibited in the pevonedistat mechanism of action both in the absence and presence of VSVΔ51 infection (FIG. 6A). Indeed, a heatmap of STAT1 regulated genes demonstrates a global reduction in RNA transcripts upon pevonedistat treatment (FIG. 6B). Cells respond to IFN cytokines via activation of the JAK (Janus activated kinase)/STAT (signal transducer and activator of transcription) pathway. Therefore, without being bound to theory, this suggests that STAT1 represents a crucial player in the IFN-1 response given its role in the interferon-stimulated growth factor complex 3 (ISGF3), along with STAT2 and interferon regulatory factor 9 (IRF9), which propagates transcription of downstream interferon-stimulated genes (ISGs) by binding to the interferon-stimulated response element.

To investigate this relationship, whole and fractionated cell lysates were probed for the components of ISGF3: STAT1, STAT2 and IRF9. The results showed that pevonedistat causes a global reduction in protein availability of ISGF3 components including STAT1 (FIG. 6C). Different concentrations of pevonedistat were unable to markedly influence phosphorylation levels of STAT1 and STAT2 in response to IFN-β treatment. On the RNA level, pevonedistat was found to significantly decrease transcription of STAT1, and several of its downstream ISGs (IRF7, MX2, IFTM1), relative to both mock and VSVΔ51 only conditions (FIG. 6D). Without wishing to be bound by theory, given that the positive induction of these components is centrally controlled by STAT1, it appears to suggest that pevonedistat suppresses the IFN-1 response by inhibiting STAT1 transcription.

Pevonedistat confers majority of its cellular effects by inhibiting neddylation activity; therefore, it was investigated whether inhibiting the neddylation pathway via silencing RNA (siRNA) could recapitulate the same viral sensitizing effects. 786-0 cells were transfected with siRNA targeting NEDD8, following which key components of the neddylation mechanism, and successful knockdown was validated at both the protein and RNA level (FIG. 6E). Transfected cells were subject to the same treatment regimen of pevonedistat pre-treatment for 4 hours, then infection with VSVΔ51-GFP (MOI 0.01). Supernatant analyzed for viral titer by plaque assay demonstrated an increase in VSVΔ51 viral titer upon NEDD8 knockdown (FIG. 6F). This finding was confirmed by representative fluorescent images taken 24 hpi, which display increased tagged GFP expression upon knockdown of NEDD8 (FIG. 6G). Moreover, analysis of mRNA from treated, transfected cells 24 hpi by qPCR demonstrated that siRNA against neddylation components on their own were able to inhibit transcription of STAT1 and downstream IRF7 (FIG. 6H). Together, these data support that pevonedistat's viral sensitizing ability is, at least in part, mediated by a neddylation-dependent suppression of STAT1 expression.

### Pevonedistat Inhibits NF-κB Independently of Neddylation to Block the Primary IFN-1 Response

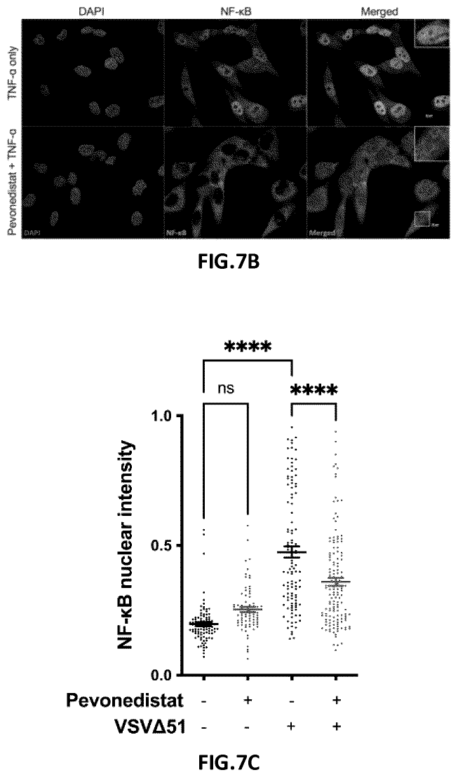

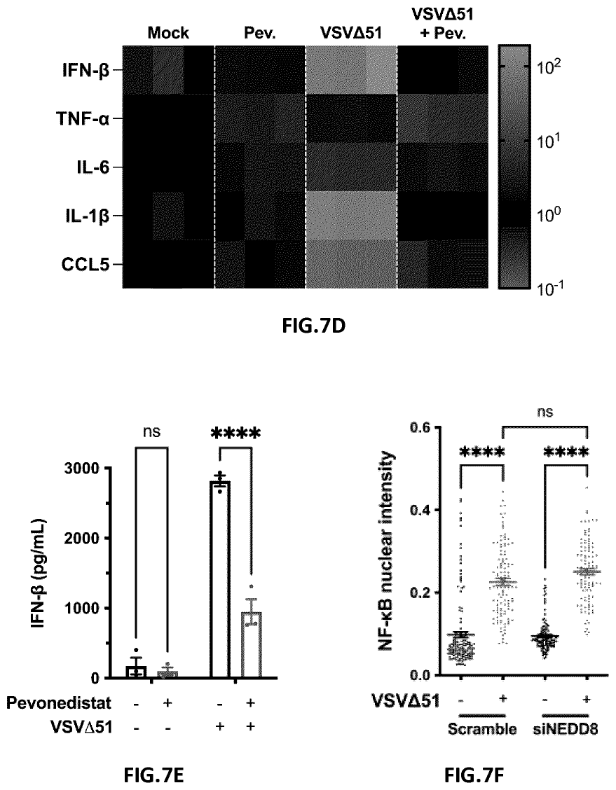

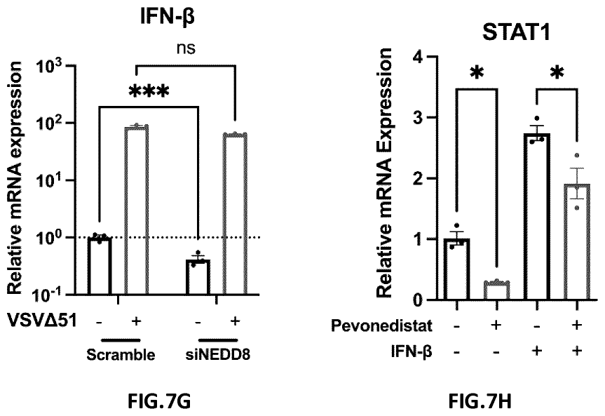

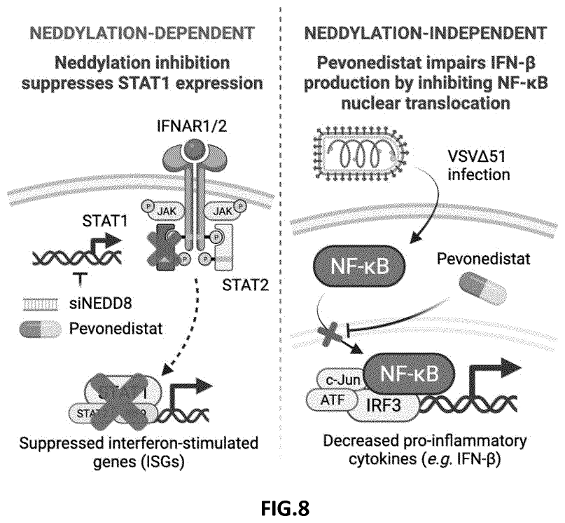

Seeing that neddylation inhibition on its own was unable to recapitulate the full viral sensitizing effect of pevonedistat, it was sought to identify a second mechanism of action. Another notable observation from the TFactS analysis was the inhibition of NF-κB transcriptional activity by pevonedistat in VSVΔ51 infected cells. Pevonedistat has previously been reported in the literature to inhibit NF-κB nuclear translocation to repress pro-inflammatory cytokine production (12). Therefore confirmation of this effect was sought in the pevonedistat+VSVΔ51 combination therapy. Indeed, nuclear/cytoplasmic fractionated lysates of 786-0 cells pre-treated with pevonedistat and infected with VSVΔ51 for 24 hours showed markedly less NF-κB protein expression, but not IRF3, in nuclear fractions compared to cells infected only with VSVΔ51 (FIG. 7A). The inhibition of NF-κB nuclear translocation in response to VSVΔ51 infection and TNF-α stimulation was also observed by immunofluorescence (FIG. 7B, FIG. 7C). As expected, treated cells showed repressed transcription of pro-inflammatory cytokines controlled by NF-κB signaling (FIG. 7D) including the central IFN-1 cytokine, IFN-β as early as 16 hpi. Quantification of IFN-β secretion 24 hpi by enzyme-linked immunosorbent assay (ELISA) followed a similar trend (FIG. 7E).

Next, it was investigated if neddylation inhibition via gene silencing would be able to recapitulate these same phenomena. The results demonstrate that siRNA targeting NEDD8 was in fact unable to abrogate induced NF-κB nuclear translocation (FIG. 7F), impair IFN-β transcription (FIG. 7G), nor impair the secretion of IFN-β in response to VSVΔ51. While not wishing to be bound by theory, together, these results suggest that pevonedistat's ability to inhibit NF-κB nuclear translocation and abrogate IFN-β production may be independently conferred from its ability to inhibit neddylation. Moreover, further investigation demonstrates that pevonedistat is still able to increase viral titers while inhibiting the transcription of STAT1 (FIG. 7H), even in the presence of exogenous IFN-3. Again, while not wishing to be bound by theory, these data support that pevonedistat's identified mechanisms in repressing STAT1 expression and inhibiting NF-κB nuclear translocation are not co-dependent.

### Example 2—Viral Sensitizing Activity by Other NAE Inhibitors

The use of viral sensitizing compounds for the enhancement of gene therapy viral vectors has been demonstrated for several other compounds. Pevonedistat (MLN4924) is a first, in-class neddylation-activating enzyme (NAE) inhibitor that has recently been identified and characterized to potentiate cancer cell infectivity to oncolytic VSVΔ51 virus infection. While Example 1 focuses on the use of pevonedistat exclusively for VSVΔ51 cancer therapy and its corresponding mechanism; in this example, the viral sensitizing ability of other established NAE inhibitors other than pevonedistat was demonstrated. The utility of these NAE inhibitors in potentiating the infection of other viral vectors, including those commonly used for gene therapy, was also demonstrated.

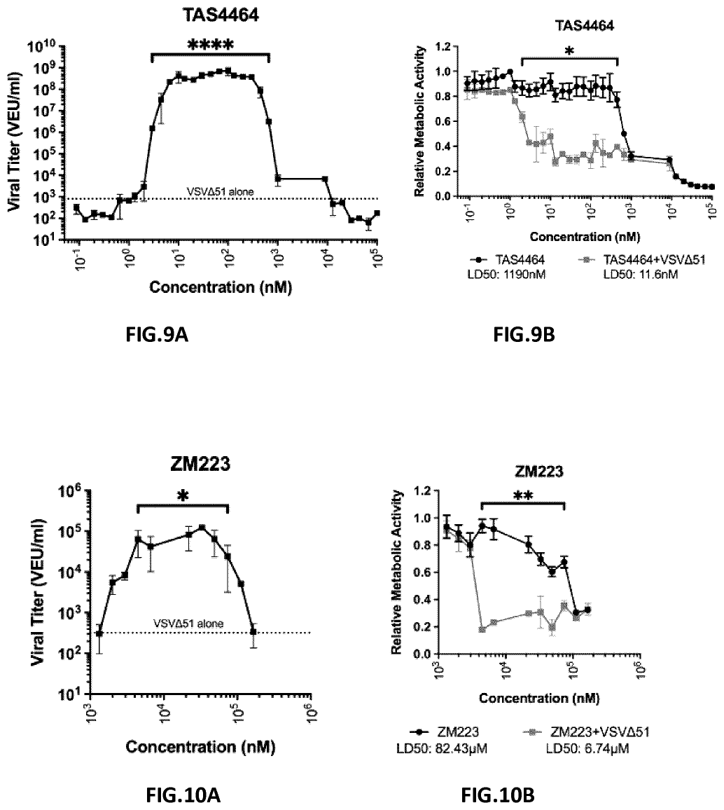

FIG. 9A, FIG. 9B, FIG. 10A and FIG. 10B show results of viral sensitizing activity by other NAE inhibitors, namely TAS4464 and ZM223. Human 786-0 renal carcinoma cells were pre-treated with a concentration range of TAS4464 or ZM223 for 4 hours, then infected with VSVΔ51 expressing firefly luciferase (VSVΔ51-FLuc), MOI 0.05. FIG. 9A, FIG. 10A: After 48 hours post infection (hpi), samples were analyzed for viral titer by high-throughput titration. The dotted line indicates baseline infection without drug (n=3, *P<0.05, ****P<0.0001 by two-way ANOVA). FIG. 9B, FIG. 10B: Cell viability was assessed using Alamar blue assay expressed relative to the uninfected, untreated condition (n=3, *P<0.05, **P<0.01 by two-way ANOVA). Without being bound to theory, the results appear to indicate that covalent NAE inhibitors confer greater viral sensitizing ability to VSVΔ51 than non-covalent NAE inhibitors.

FIG. 9A, FIG. 9B, FIG. 10A and FIG. 10B show data on two commercially available NAE inhibitors: TAS4464 (covalent) vs. ZM223 (non-covalent). Like pevonedistat (covalent), it was demonstrated that TAS4464 confers potent (>log 5 fold-change VEU) sensitizing activity to VSVΔ51 infectivity and enhanced oncolysis across a broad range of concentrations in the nanomolar range. While ZM223 (non-covalent) demonstrates some viral sensitizing activity (˜log 2 fold-change VEU) for infectivity, it is notably not as dramatic as the covalent inhibitors. However, its ability to sensitize cells to VSVΔ51 appears to persist, which may point towards a more neddylation-dependent mechanism for the increased oncolysis.

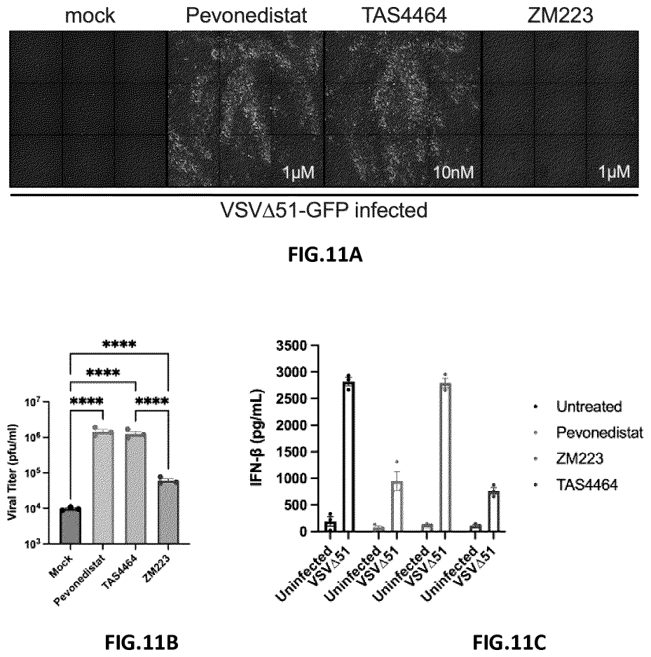

FIG. 11A, FIG. 11B, and FIG. 11C show results of viral sensitizing ability to VSVΔ51 via IFN-1 repression for covalent and non-covalent NAE inhibitors, where 786-0 renal carcinoma cells were pre-treated with the indicated dose of pevonedistat, TAS4464 or ZM223 for 4 hours, then infected with VSVΔ51-GFP (MOI 0.01) for 24 hours. FIG. 11A: Representative fluorescent images were taken. FIG. 11B: Supernatant was analyzed for viral titer by plaque assay (n=3, ****P<0.0001 by one-way ANOVA). FIG. 11C: Supernatant was measured for IFN-β secretion by ELISA (n=3, ****P<0.0001 by one-way ANOVA).

In support of FIG. 9A, FIG. 9B, FIG. 10A and FIG. 10B, the covalent NAE inhibitors (pevonedistat, TAS4664) clearly demonstrate increased VSVΔ51 infectivity as supported by the increased fluorescent signal in FIG. 11A, which is subsequently confirmed by plaque assay in FIG. 11B. While ZM223 confers some viral sensitizing activity, it is incomparable to the impact by the covalent NAE inhibitors. This viral sensitizing effect was shown above to be caused by repression of the antiviral, IFN-1 response such as IFN-3. Accordingly, the covalent inhibitors significantly repress IFN-β secretion while ZM223, the non-covalent inhibitors, is unable to do the same, as supported in FIG. 11C.

### Example 3—Viral Sensitizing Activity of Pevonedistat in Other Viral Vectors

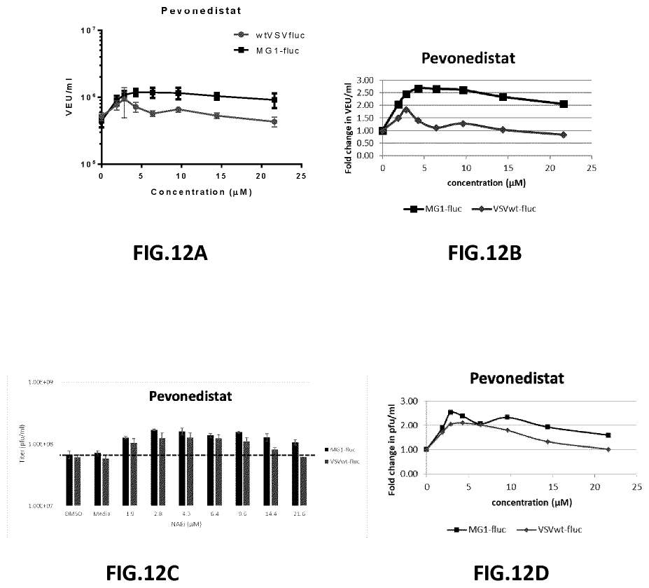

Results of viral sensitizing activity of pevonedistat in other viral vectors are shown in FIG. 12A, FIG. 12B, FIG. 12C and FIG. 12D. Vero cells were treated with a range of concentrations of pevonedistat as indicated for 4 hours, then infected with either VSV-wild type-FLuc (MOI 0.001) or MG-1 (MOI 0.01). FIG. 12A, FIG. 12B: Samples were assessed for viral titer by high-throughput titration, or FIG. 12C, FIG. 12D: by plaque assay.

Without being bound to theory, these experiments demonstrate the ability for pevonedistat to not only sensitize the attenuated VSVΔ51 oncolytic virus, but to facilitate production of wild-type VSV and MG-1 in Vero cells deficient of IFN-1 response.

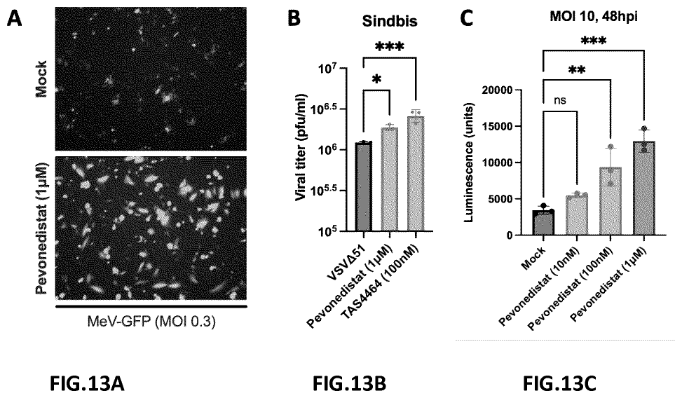

FIG. 13A, FIG. 13B, FIG. 13C show results of NAE inhibitors sensitization of cancer cells to other viral vectors. FIG. 13A: 786-0 cells were pre-treated with pevonedistat (1 μM) for 4 hours, then infected with measles (MeV) tagged with GFP (MOI 0.3). Fluorescent images were taken 48 hpi. FIG. 13B: 786-0 cells were pre-treated with pevonedistat (100 nM or 1 μM) for 4 hours then infected with Sindbis (MOI 0.01). After 48 hpi, supernatants were analyzed for viral titer by plaque assay (n=3, *P<0.05, ***P<0.001 by one-way ANOVA). FIG. 13C: Human lung A549 adenocarinoma cells were pre-treated with pevonedistat (10 nM, 100 nM, 1 μM) for 4 hours, then infected with adenovirus serotype 5 (Ad5) tagged with FLuc at MOI 10. After 48 hpi, supernatants were assessed for luminescence activity indicative of Ad5 quantity (n=3, **P<0.01, ***P<0.001 by one-way ANOVA).

This demonstrates the utility of NAE inhibitors in potentiating other viral vectors including those commonly used for gene therapy. MeV and SinV are two other oncolytic platforms for exploration with this novel class of viral sensitizing molecules, while this preliminary data with Ad5, a common gene therapy vector, opens the possibility for the utility of NAE inhibitors in the gene therapy space.

### Example 4—Viral Sensitizing Activity of Pevonedistat in Other Tumor Contexts

To show that the viral sensitizing activity of pevonedistat is not a cell-line specific phenomenon, the findings described in Examples 1-3 were validated in another human cell model, the HT29 colon adenocarcinoma cell line.

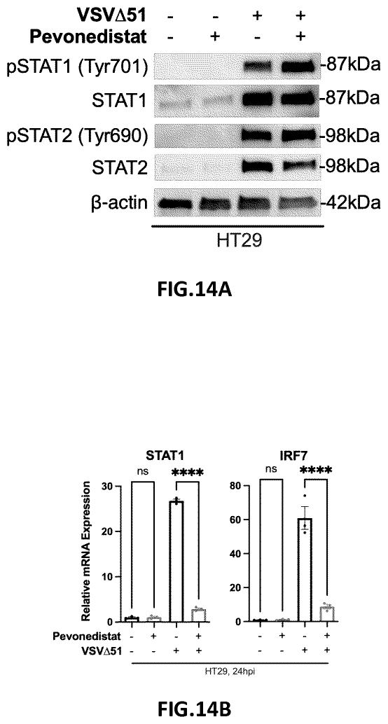

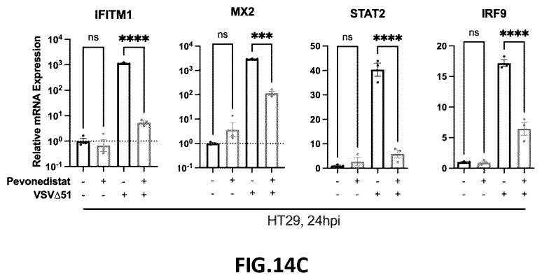

The levels of phosphorylated and total STAT1 and STAT2 in whole cell HT29 lysate treated with pevonedistat and VSVΔ51 were investigated. Indeed, similar to 786-0, total protein levels of STAT1 and STAT2 were decreased (FIG. 14A). It was previously demonstrated that downstream RNA levels of IFN-1 effectors were also decreased, a finding confirmed in FIG. 14B and FIG. 14C.

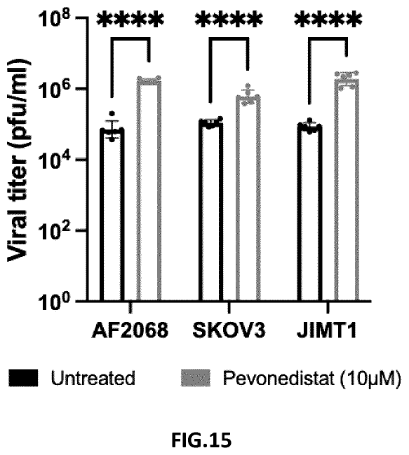

To add additional tumour contexts in which pevonedistat can enhance VSVΔ51, an ex vivo xenograft model was pursued. Different human tumour cell lines were implanted into nude-mice and allowed to progress to sufficient size before being extracted and tested with the drug combination ex vivo. As shown in native murine cores, pevonedistat is able to increase the viral titer of VSVΔ51 by plaque assay (FIG. 15).

### Example 5—RNAi Mediated Knockdown of Neddylation Confers Partial Enhancing Impact

The finding that RNAi mediated knockdown of neddylation conferred partial, but not full viral enhancing impact. UBA3 is a key component to the neddylation activating enzyme (NAE), which is the primary target of pevonedistat, was validated.

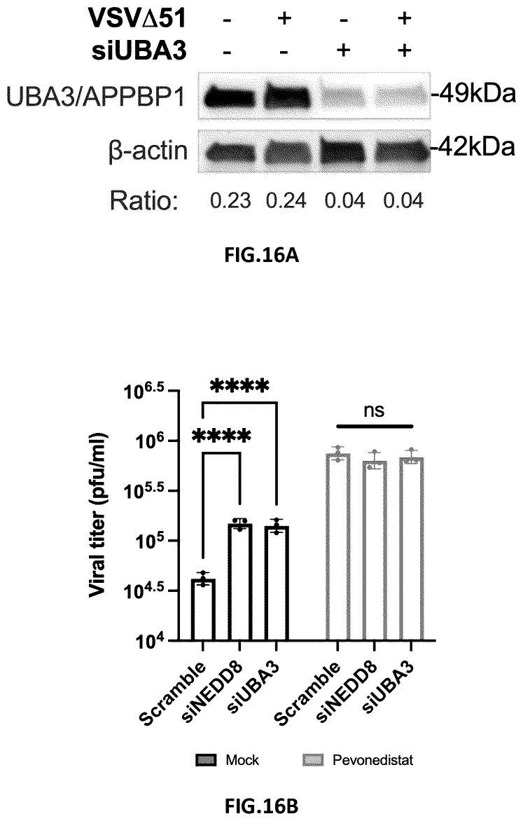

Knockdown of UBA3 effectively inhibits all cellular neddylation processes. The ability to knockdown UBA3 protein expression was first confirmed by western blot (FIG. 16A). Infected cells were transfected with siRNA against UBA3 to confirm partial viral enhancement as demonstrated by siRNA against NEDD8. Indeed, both conferred a similar level of partial, but not full VSVΔ51 viral enhancement confirming that inhibition of any part of the neddylation process impaired the anti-viral response (FIG. 16B).

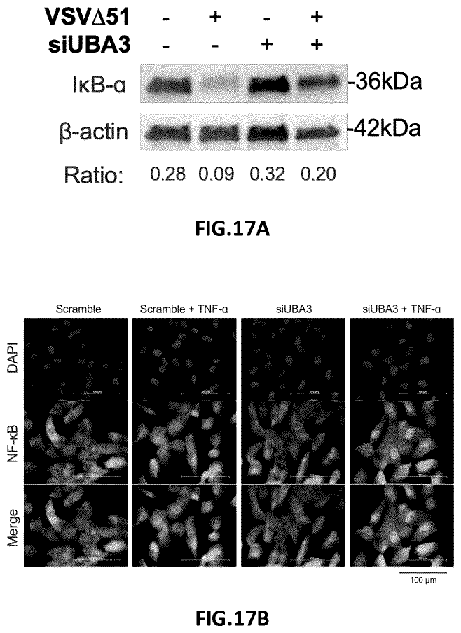

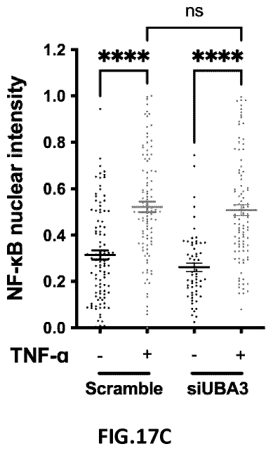

To further confirm the proposed mechanism of neddylation-independent inhibition of NF-κB nuclear translocation, it was shown that knockdown of UBA3 could impact levels of IκB-α (FIG. 17A), but not inhibit nuclear translocation of NF-κB in response to induction by TNF-α (FIG. 17B, FIG. 17C). This confirms the previous findings with siRNA against NEDD8.

### Example 6—Further In Vivo Studies Showing Viral Sensitizing Activity of Pevonedistat In Vivo in Melanoma B16 Tumors and Ovarian ID8-Tp53−/− (F3) Cells

The in vivo data set out in Example 1 was expanded upon.

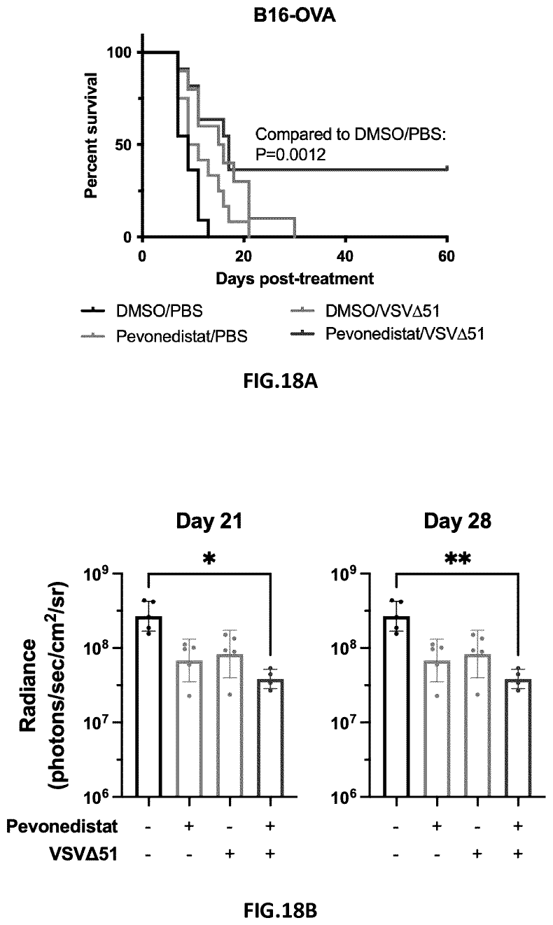

In the more aggressive 4T1 model, the pevonedistat+VSVΔ51 combination also significantly prolonged survival compared to all other conditions (FIG. 18A; P=0.0009 vs. Pevonedistat group, P=0.04 vs. VSVΔ51 group). These results demonstrate that pevonedistat in combination with VSVΔ51 therapy confers improved therapeutic benefit when compared to mice receiving placebo or either monotherapy.

The efficacy of systemically administered pevonedistat combination therapy in a disseminated model of intra-abdominal cancer was also studied. Murine ID8-Tp53−/− (F3) ovarian cancer cells were tagged with firefly luciferase, injected into the peritoneum of C57BL/6 mice, and monitored using a live luminescence imaging system. When sufficient tumor burden was achieved (7 d post implantation), mice were injected intraperitoneally with pevonedistat (90 mg/kg), then 1×10⁸ pfu VSVΔ51 4 hours later. Compared to mice receiving placebo treatment, mice receiving combinational treatment demonstrated significantly reduced tumor burden as measured by luciferase signal, which was notably not significant for either monotherapy (FIG. 18B).

### Example 7—Pevonedistat Enhances AAV Virus Production

The effect of pevonedistat on AAV2-Fluc production was assessed by a functional titre assay. HEK293 cells were seeded in 96-well microplates, which were treated at 70% confluence. The cells were co-transfected with pHelper, pAAV ITR-Fluc vector and pAAV Rep-Cap genes in 1:1:1 molar ratio normalized to the plasmid size. 60 min post transfection the cells were treated with MLN4924 or vehicle control—(DMSO).

At 72 h post treatment, the cells were lysed by cycle of three freeze-thawing where the microplates were placed in the −80 c freezer for 1 hour followed by 20 min incubation in 37c water bath.

The pevonedistat-treated, DMSO vehicle control-treated as well as untreated transfected (baseline) samples (n=12) lysates from above were diluted 1:10 in serum-free DMEM prior to transduction in un-treated HEK293 cells for virus quantification.

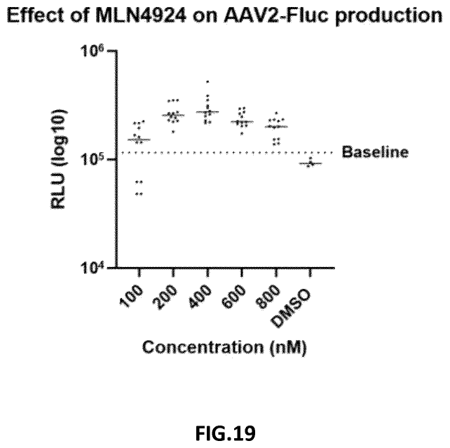

The transgene signal was quantified 72 h post-transduction using firefly luciferase assay measuring luminescence and compared to a standard curve with known quantities of AAV2-Fluc. As shown in FIG. 19, viral signal was enhanced up to 2.6 fold over a range of concentrations, optimally between 200-400 nM, respectively. Overall this data shows the ability of the NAE inhibitor pevonedistat to enhance the production of a non-replicating AAV vector produced from a plasmid-transfection based reverse genetics system.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### FULL CITATIONS FOR DOCUMENTS REFERENCED IN THE APPLICATION

1. M. D. Fullerton, F. Hakimuddin, A. Bonen, M. Bakovic, The Development of a Metabolic Disease Phenotype in CTP:Phosphoethanolamine Cytidylyltransferase-deficient Mice. J. Biol. Chem. 284, 25704-25713 (2009).
2. J.-S. Diallo, M. Vshs-Koskela, F. Le Boeuf, J. Bell, in Oncolytic Viruses, Methods in Molecular Biology. D. H. Kirn, T.-C. Liu, S. H. Thorne, Eds. (Humana Press, Totowa, NJ, 2012), vol. 797, pp. 127-140.
3. P. T. Sobol, J. E. Boudreau, K. Stephenson, Y. Wan, B. D. Lichty, K. L. Mossman, Adaptive Antiviral Immunity Is a Determinant of the Therapeutic Success of Oncolytic Virotherapy. Mol. Ther. 19, 335-344 (2011).
4. C. J. Breitbach, J. Burke, D. Jonker, J. Stephenson, A. R. Haas, L. Q. M. Chow, J. Nieva, T.-H. Hwang, A. Moon, R. Patt, A. Pelusio, F. Le Boeuf, J. Burns, L. Evgin, N. De Silva, S. Cvancic, T. Robertson, J.-E. Je, Y.-S. Lee, K. Parato, J.-S. Diallo, A. Fenster, M. Daneshmand, J. C. Bell, D. H. Kirn, Intravenous delivery of a multi-mechanistic cancer-targeted oncolytic poxvirus in humans. Nature 477, 99-102 (2011).
5. J. Brun, D. McManus, C. Lefebvre, K. Hu, T. Falls, H. Atkins, J. C. Bell, J. A. McCart, D. Mahoney, D. F. Stojdl. Identification of genetically modified Maraba virus as an oncolytic rhabdovirus. Mol Ther. 18, 1440-9 (2010).
6. V. Garcia, R. Krishnan, C. Davis, C. Batenchuk, F. Le Boeuf, H. Abdelbary, J.-S. Diallo, High-throughput Titration of Luciferase-expressing Recombinant Viruses. J. Vis. Exp., 51890 (2014).
7. N. L. Bray, H. Pimentel, P. Melsted, L. Pachter, Near-optimal probabilistic RNA-seq quantification. Nat. Biotechnol. 34, 525-527 (2016).
8. H. Pimentel, N. L. Bray, S. Puente, P. Melsted, L. Pachter, Differential analysis of RNA-seq incorporating quantification uncertainty. Nat. Methods 14, 687-690 (2017).
9. E. Eden, R. Navon, I. Steinfeld, D. Lipson, Z. Yakhini, GOrilla: a tool for discovery and visualization of enriched GO terms in ranked gene lists. BMC Bioinformatics 10, 48 (2009).
10. A. Essaghir, F. Toffalini, L. Knoops, A. Kallin, J. van Helden, J.-B. Demoulin, Transcription factor regulation can be accurately predicted from the presence of target gene signatures in microarray gene expression data. Nucleic Acids Res. 38, e120-e120 (2010).
11. S. A. Felt, M. J. Moerdyk-Schauwecker, V. Z. Grdzelishvili, Induction of apoptosis in pancreatic cancer cells by vesicular stomatitis virus. Virology 474, 163-173 (2015).
12. J. C. Godbersen, L. A. Humphries, O. V. Danilova, P. E. Kebbekus, J. R. Brown, A. Eastman, A. V. Danilov, The Nedd8-Activating Enzyme Inhibitor MLN4924 Thwarts Microenvironment-Driven NF-κB Activation and Induces Apoptosis in Chronic Lymphocytic Leukemia B Cells. Clin. Cancer Res. 20, 1576-1589 (2014).
13. Pieter R. Cullis, Michael J. Hope. Lipid Nanoparticle Systems for Enabling Gene Therapies. Mol Ther. 25(7): 1467-1475 (2017).
14. Michael J. Mitchell, Margaret M. Billingsley, Rebecca M. Haley, Marissa E. Wechsler, Nicholas A. Peppas, Robert Langer, Engineering precision nanoparticles for drug delivery. Nat Rev Drug Discov. 20(2): 101-124 (2021).

1. A method of increasing production of a virus by a cell comprising administering a neddylation-activating enzyme (NAE) inhibitor, or a salt, solvate and/or prodrug thereof to the cell.

2. The method of claim 1, comprising transfecting the cell with one or more plasmids encoding one or more components of a virus and contacting the transfected cell with the NAE inhibitor, or salt, solvate and/or prodrug thereof.

3. The method of claim 1 or 2, wherein the cell is a viral production cell.

4. The method of claim 3, wherein the viral production cell is a Vero, HEK-293, VPC 1.0, VPC 2.0, EB-66, EbX, PER, C6, AGE1.CR, UMNSAH-DF1, CEF, MRC-5, WI-38, BHK21, Hela, A549 or sf9 cell.

5. The method of any one of claims 1 to 4, wherein the virus produced by the cell is a non-replicating virus.

6. The method of claim 5, wherein the virus produced by the cell is an adenovirus (Ad), an adeno-associated virus (AAV) or lentivirus (LV).

7. The method of claim 5, wherein the virus produced by the cell is a non-replicating adeno-associated virus (AAV).

8. The method of claim 5, wherein the virus produced by the cell is an oncolytic virus, gene therapy vector or a vaccine.

9. A method of increasing permissiveness of a cell to a virus, comprising administering an effective amount of a neddylation-activating enzyme (NAE) inhibitor, or a salt, solvate and/or prodrug thereof, to the cell.

10. The method of claim 9, wherein the NAE inhibitor, or salt, solvate and/or prodrug thereof, is administered to the cell before, after and/or concurrently with the virus.

11. The method of claim 10, wherein the NAE inhibitor, or salt, solvate and/or prodrug thereof, is administered to the cell before the virus is administered to the cell.

12. The method of any one of claims 9 to 11, wherein the permissiveness of the cell to the virus is increased 1.1 fold or more compared to permissiveness of the cell, or a comparable cell, prior to the method or in the absence of the method.

13. The method of any one of claims 1 to 12, wherein the virus is a therapeutic virus.

14. The method of any one of claims 1 to 13, wherein the virus is an interferon (IFN)-sensitive virus.

15. The method of any one of claims 1 to 14, wherein the virus is an attenuated virus, a genetically modified virus, a non-replicating virus, a gene therapy vector, or an oncolytic virus.

16. The method of any one of claims 1 to 15, wherein the virus is a herpes simplex virus (HSV) viral vector.

17. The method of any one of claims 1 to 16, wherein the virus is an adenovirus.

18. The method of claim 15, wherein the gene therapy viral vector is Ad5, Ad3, Ad11, Ad35, canine Ad2, chimp Ad26, chimp AdOx1, or recombinant serotypes therein, AAV serotypes 1-9 or recombinant serotypes therein, Lentivirus, gamma-retrovirus, Annellovirus, or Baculovirus.

19. The method of any one of claims 1 to 15, wherein the virus is a rhabdovirus, a togavirus, or an orthomyxovirus.

20. The method of claim 18, wherein the rhabdovirus is vesicular stomatitis virus (VSV), engineered mutants of VSV (VSVΔ51), an oncolytic non-VSV rhabdovirus, or a recombinant oncolytic non-VSV rhabdovirus encoding one or more of rhabdoviral N, P, M, G and/or L protein, or variant thereof including chimeras and fusion proteins thereof, having an amino acid identity of at least or at most 20, 30, 40, 50, 60, 65, 70, 75, 80, 85, 90, 92, 94, 96, 98, 99, 100%, including all ranges and percentages there between, to the N, P, M, G and/or L protein of Arajas virus, Chandipura virus, Cocal virus, Isfahan virus, Maraba virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Aimpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus.

21. The method of claim 19, wherein the togavirus is sindbis, semliki forest virus or M1 virus.

22. The method of claim 19, wherein the orthomyxovirus is influenza A, influenza B, influenza C, influenza D, isavirus, thogotovirus or quanranjavirus.

23. The method of claim 15, wherein the virus is a component of a vaccine.

24. The method of claim 23, wherein the component of the vaccine is a live attenuated vaccine selected from measles, mumps, rubella, rotavirus, chickenpox, and yellow fever, or a viral vector vaccine encoding a vaccine antigen transgene selected from rVSVΔG-ZEBOV-GP (Ervebo) and ChadOx1-S (Vaxzevria).

25. The method of any one of claims 9 to 14, wherein the virus comprises a non-replicating viral vector.

26. The method of claim 25, wherein the viral vector is an adenovirus (Ad), an adeno-associated virus (AAV) or lentivirus (LV).

27. The method of any one of claims 9 to 26, wherein the cell is a eukaryotic cell or a prokaryotic cell.

28. The method of any one of claims 9 to 26, wherein the cell is a human cell or a mammalian cell.

29. The method of any one of claims 9 to 26, wherein the cell is in vivo, ex vivo or in vitro.

30. The method of any one of claims 9 to 26, wherein the cell is a cell in subject.

31. The method of any one of claims 9 to 26, wherein the cell is a cell line or a cell culture.

32. A method of increasing permissiveness of a cell to genetic material encoding components of a virus, comprising administering an effective amount of a neddylation-activating enzyme (NAE) inhibitor, or a salt, solvate and/or prodrug thereof, to the cell in combination with provision of the genetic material encoding components of a virus to the cell.

33. The method of claim 32, wherein the provision of the genetic material encoding components of a virus to the cell is prior, concurrent with or after administration of the effective amount of the NAE inhibitor, or salt, solvate and/or prodrug thereof.

34. The method of claim 32 or 33, wherein the genetic material encoding components of a virus are nucleic acids, or chemically modified variants thereof, comprising viral and/or viral-like sequences.

35. The method of any one of claims 32 to 34, wherein the genetic material encoding components of a virus encode viral proteins, viral-like proteins and/or functional sequences.

36. The method of any one of claims 32 to 35, wherein the genetic material encoding components of a virus is delivered directly to the cell or is delivered in a carrier.

37. The method of any one of claims 32 to 36, wherein the genetic material encoding components of a virus is comprised in a plasmid.

38. The method of claim 37, wherein a lentivirus, gamma-Retrovirus, or AAV is produced following transfection of the plasmid encoding lentivirus, gamma-Retrovirus, or AAV viral or viral-like sequences into the cell.

39. A method of treating a disease, disorder or condition by increasing permissiveness of a cell to a virus comprising administering a therapeutically effective amount of a neddylation-activating enzyme (NAE) inhibitor, or a salt, solvate and/or prodrug thereof and the virus or genetic material encoding the virus to a subject in need thereof.

40. The method of claim 39, wherein the NAE inhibitor, or salt, solvate and/or prodrug thereof is administered to the cell before, after and/or concurrently with the virus that treats the disease, disorder or condition or genetic material encoding the virus that treats the disease, disorder or condition.

41. The method of claim 39 or 40, wherein the NAE inhibitor, or salt, solvate and/or prodrug thereof allows a lower amount of the virus or genetic material encoding components to be used to treat the disease, disorder or condition.

42. The method of any one of claims 39 to 41, wherein the disease, disorder or condition is cancer or a tumor.

43. The method of any one of claims 39 to 42, wherein the virus is an oncolytic virus.

44. The method of claim 43, wherein the oncolytic virus is a virus that preferentially infects and lyses cancer or tumor cells as compared to non-cancer or normal cells.

45. The method of claim 43 or 44, wherein the oncolytic virus is talimogene laherparepvec (T-VEC), Delytact, Maraba MG-1, or vesicular stomatitis virus (VSVΔ51). In some embodiments, the oncolytic virus is a Newcastle Disease Virus (NDV), measles virus, (MeV), parvovirus H1 (ParvOryx), M1 virus, poliovirus, reovirus, Myxomavirus, or Sindbis virus (SinV).

46. The method of any one of claims 42 to 45, wherein the cancer is lymphoblastic leukemia, myeloid leukemia, adrenocortical carcinoma, AIDS-related cancer, AIDS-related lymphoma, anal cancer, appendix cancer, astrocytoma, atypical teratoid/rhabdoid tumor, basal cell carcinoma, bile duct cancer, bladder cancer, bone cancer, osteosarcoma, malignant fibrous histiocytoma, brain stem glioma, brain tumor, cerebellar astrocytoma, cerebral astrocytoma/malignant glioma, craniopharyngioma, ependymoblastoma, medulloblastoma, pineal parenchymal tumors of intermediate differentiation, supratentorial primitive neuroectodermal tumors and pineoblastoma, visual pathway and hypothalamic glioma, spinal cord tumors, breast cancer, bronchial tumors, Burkitt lymphoma, carcinoid tumor, central nervous system lymphoma, cervical cancer, chordoma, chronic lymphocytic leukemia, chronic myelogenous leukemia, chronic myeloproliferative disorders, colon cancer, cutaneous T-Cell lymphoma, embryonal tumors, endometrial cancer, ependymoblastoma, ependymoma, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), gastrointestinal stromal cell tumor, germ cell tumors, extracranial, extragonadal, ovarian, gestational trophoblastic tumor, glioma, hairy cell leukemia, head and neck cancer, hepatocellular (Liver) cancer, histiocytosis, Langerhans cell cancer, Hodgkin lymphoma, hypopharyngeal cancer, islet cell tumors, Kaposi sarcoma, kidney cancer, laryngeal cancer, lymphocytic leukemia, hairy cell leukemia, lip and oral cavity cancer, liver cancer, non-small cell lung cancer, small cell lung cancer, Hodgkin lymphoma, non-Hodgkin lymphoma, malignant fibrous hi stiocytoma of bone and osteosarcoma, medulloblastoma, medulloepithelioma, melanoma, intraocular melanoma, Merkel cell carcinoma, mesothelioma, metastatic squamous neck cancer, mouth cancer, multiple endocrine neoplasia syndrome, multiple myeloma/plasma cell neoplasm, nasal cavity and paranasal sinus cancer, nasopharyngeal cancer, neuroblastoma, oral cancer, oropharyngeal cancer, ovarian cancer, pancreatic cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, renal pelvis and ureter cancer, transitional cell cancer, respiratory tract carcinoma, retinoblastoma, rhabdomyosarcoma, salivary gland cancer, uterine sarcoma, skin cancer, Merkel cell skin carcinoma, small intestine cancer, soft tissue sarcoma, squamous cell carcinoma, squamous neck cancer, stomach (Gastric) cancer, supratentorial primitive neuroectodermal tumors, T-Cell lymphoma, testicular cancer, throat cancer, thymoma and thymic carcinoma, thyroid cancer, trophoblastic tumor, urethral cancer, uterine cancer, endometrial cancer, uterine sarcoma, vaginal cancer, vulvar cancer, or Wilms tumor.

47. The method of any one of claims 42 to 45, wherein the cancer is colon cancer, breast cancer, rectal cancer, lung cancer, a leukemia, cervical cancer, sarcoma, melanoma, pancreatic cancer and/or ovarian cancer.

48. The method of any one of claims 42 to 47, wherein the subject is a mammal.

49. The method of any one of claims 42 to 47, wherein the subject is a human.

50. The method of any one of claims 42 to 49, wherein the cell is a cancer cell, a tumor cell or an immortalized cell.

51. The method of any one of claims 42 to 49, wherein the cell is one or more types of immortalized cells in vitro or in vivo from a cell, cell line, tissue or organism selected from human, rat, mouse, cat, dog, pig, primate, horse, Vero, HEK-293 cells, VPC 1.0, VPC 2.0, EB-66 cells, EbX cells, PER. C6 cells, AGE1.CR, Agel.0 S, Agel.HN, Agel.RO, Q0R2/2E11, UMNSAH-DF1, CHO, hybridoma cells, sf9 cells, or R⁴ cells.

52. The method of any one of claims 42 to 49, wherein the cell is a tumor forming cells selected from 293-T cells, BHK21 cells, and MDCK cells.

53. A method of increasing the oncolytic activity of a virus comprising administering a therapeutically effective amount of a NAE inhibitor, or a salt, solvate and/or prodrug thereof and an oncolytic virus to a subject or cell in need thereof.

54. A method of treating a disease, disorder or condition by gene therapy comprising administering a therapeutically effective amount of a NAE inhibitor, or a salt, solvate and/or prodrug thereof and a gene therapy vector to a subject or cell in need thereof.

55. A method of increasing transduction of a virus into a cell comprising administering a NAE inhibitor, or a salt, solvate and/or prodrug thereof and the virus to the cell.

56. A method of increasing virally-encoded transgene expression comprising administering a NAE inhibitor, or a salt, solvate and/or prodrug thereof and the virus to a cell.

57. A method of increasing virus growth and/or virus spread in cells comprising administering a NAE inhibitor, or a salt, solvate and/or prodrug thereof to the cells in combination with provision of the virus to the cells.

58. The method of claim 57, wherein the provision of the virus to the cells is prior, concurrent with or after administration of the NAE inhibitor, or salt, solvate and/or prodrug thereof.

59. The method of any one of claims 1 to 58, wherein the NAE inhibitor is a covalent NAE inhibitor.

60. The method of any one of claims 1 to 58, wherein the NAE inhibitor is MLN4924, TAS4464 or ZM223, or a salt, solvate and/or prodrug thereof, or a combination thereof.

61. The method of any one of claims 1 to 58, wherein the NAE inhibitor is a compound of Formula (I): 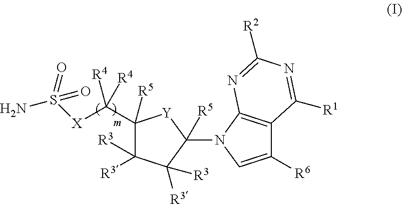
or a salt, solvate and/or prodrug thereof, or a combination thereof, wherein:
X is CH₂, CHF, CF₂, NH or O;
Y is O, S or CH₂;
R¹ is H, Cl, Br, F, I, NR⁷R⁸, R⁹, SH, SCH₃, SR¹⁰, OH, OCH₃ or OR¹⁰;
R² is H, Cl, Br, F, I, N(R⁸), CN, OR, SR, or an optionally substituted C₁₋₄alkyl;
each R³ is independently H, F, C₁₋₄alkyl or C₁₋₄ fluoroalkyl;
each R^{3′} is independently H, CN, N₃, OH, OR¹¹, NH₂, NHR¹¹, NHCO₂R¹¹, NHC(O)R¹¹, C(O)NHR¹¹, OC(O)NHR¹¹, OC(O)R¹¹, OC(O)OR¹¹, C₁₋₄fluoroalkyl, or C₁₋₄alkyl optionally substituted with one or two substituents each being independently OR¹², NR¹³R¹⁴, CO₂R¹² or CONR¹³R¹⁴
each R⁴ is independently H, F, C₁₋₄alkyl or C₁₋₄fluoroalkyl; or two R⁴ taken together with the carbon to which they are attached form a 3- to 6-membered ring; or two R⁴ form ═O;
each R⁵ is independently H or C₁₋₄alkyl;
or one R⁴ taken with R⁵ on the adjacent carbon form with the intervening carbon atoms a 3- to 6-membered ring;
R⁶ is H, halo or an optionally substituted C₁₋₄alkyl;
R⁷ is an optionally substituted C₆₋₁₀aryl, C₅₋₁₀heteroaryl or C₃₋₁₀heterocyclyl;
R⁸ is H or C₁₋₄alkyl;
R⁹ is VZR¹⁵, VZR¹⁶, R¹⁷, or an optionally substituted C₁₋₁₀alkyl, C₆₋₁₀aryl, C₅₋₁₀heteroaryl or C₃₋₁₀heterocyclyl, wherein the heteroaryl is attached at a carbon atom;
R¹⁰ is an unsubstituted C₂₋₁₀alkyl, a substituted C₁₋₁₀alkyl, or optionally substituted C₆₋₁₀aryl, C₅₋₁₀heteroaryl or C₃₋₁₀heterocyclyl;
R¹¹ is an optionally substituted C₁₋₁₀alkyl, C₆₋₁₀aryl, C₅₋₁₀heteroaryl or C₃₋₁₀heterocyclyl;
R¹² is H, C₁₋₄alkyl, C₁₋₄fluoroalkyl, or an optionally substituted C₆₋₁₀aryl or C₁₋₄alkyleneC₆₋₁₀aryl;
R¹³ is H, C₁₋₄alkyl, C₁₋₄fluoroalkyl, or an optionally substituted C₁₋₄alkyleneC₆₋₁₀aryl;
R¹⁴ is H, C₁₋₄alkyl, C₁₋₄fluoroalkyl, an optionally substituted C₁₋₄alkyleneC₆₋₁₀aryl or an optionally substituted 5- or 6-membered aryl, heteroaryl or heterocyclyl;
R¹⁵ is an optionally substituted C₆₋₁₀aryl, C₅₋₁₀heteroaryl, C₃₋₁₀heterocyclyl or C₃₋₁₀cycloalkyl;
R¹⁶ is halo, NO₂, CN, OR¹⁸, SR¹⁹, N(R²⁰)₂, N(R²⁰)C(O)R¹⁹, N(R²⁰)C(O)NR²⁰, N(R²⁰)CO₂R¹⁸, OCO₂R¹⁸, OC(O)N(R²⁰)₂, OC(O)R¹⁸, N(R²⁰)N(R²⁰)₂, N(R²⁰)OR¹⁹, N(R²⁰)SO₂R¹⁹, N(R²⁰)SO₂N(R²⁰)₂, CR¹⁸═C(R¹⁸)₂, —C≡CR¹⁸, S(O)R¹⁹, SO₂R¹⁹, SO₂N(R²⁰)₂, CR¹⁸═NOR¹⁸, CO₂R¹⁸, C(O)C(O)R¹⁸, C(O)R¹⁸, C(O)N(R²⁰)₂, C(═NR²⁰)N(R²⁰)₂ or C(═NR²⁰)OR¹⁸,
R¹⁷ is NO₂, CN, SO₂R¹⁹, SO₂N(R²⁰)₂, SO₂, N(R²⁰)₂, C(R¹⁸)═NOR¹⁸, N(R²⁰)C(O)R¹⁹, N(R²⁰)C(O)N(R²⁰)₂, OCO₂R¹⁸, OC(O)N(R²⁰)₂, OC(O)R¹⁸, CO₂R¹⁸, C(O)C(O)R¹⁸, C(O)R¹⁸, C(O)N(R²⁰)₂, C(═NR²⁰)N(R²⁰)₂, C(═NR²⁰)OR¹⁸, N(R²⁰)N(R²⁰)₂, N(R²⁰)OR¹⁹, N(R²⁰)SO₂R¹⁹ or N(R²⁰)SO₂N(R²⁰)₂;
V is —SO₂—, —SO—, —CO₂—, —C(O)—, —C(NR¹⁸)═N—, —C(═NR¹⁸)N(R¹⁸)—, —C(OR¹⁰)═N—, —C(O)N(R¹⁸)—, —N(R¹⁸)C(O)—, —N(R¹⁸)C(O)N(R¹⁸)—, —N(R¹⁸)SO₂—, —N(R¹⁸)SO₂N(R¹⁸)—, —N(R¹⁸)CO₂—, —SO₂N(R¹⁸)—, —OC(O)—, —OCO₂—, —OC(O)N(R¹⁸)— or —N(R¹⁸)N(R¹⁸)—;
Z is an optionally substituted alkylene, optionally interrupted with —CR¹⁸CR¹⁸—, —C—O—, —S—, —C≡C—, —N(R¹⁸)—, —N(R¹⁸)C(O)—, —N(R¹⁸)CO₂—, —C(O)N(R¹⁸)—, —C(O)—, —C(O)—C(O)—, —CO₂—, —OC(O)—, —OCO₂—, —N(R¹⁸)C(O)N(R¹⁸)—, —N(R¹⁸)N(R¹⁸)—, —OC(O)N(R¹⁸)—, —SO₂—, —SO—, —N(R¹⁸)SO₂— or —SO₂N(R¹⁸)—;
each R¹⁸ is independently H or an optionally substituted C₁₋₁₀alkyl, C₆₋₁₀aryl, C₅₋₁₀heteroaryl or C₃₋₁₀heterocyclyl;
each R¹⁹ is independently an optionally substituted C₁₋₁₀alkyl or C₆₋₁₀aryl;
each R²⁰ is independently an optionally substituted C₁₋₁₀alkyl, C₆₋₁₀aryl, C₅₋₁₀heteroaryl or C₃₋₁₀heterocyclyl, or two R²⁰ on the same nitrogen atom are taken together with the nitrogen atom to form an optionally substituted 5- to 8-membered heterocyclyl ring having zero to two additional heteroatoms selected from N, O and S;
wherein each recitation of optionally substituted aryl refers to one or more substituents independently selected from halogen, NO₂, CN, R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂;
wherein each recitation of optionally substituted heteroaryl refers to one or more substituents on an unsaturated carbon atom independently selected from halogen, —NO₂, —CN, R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂; or on a substitutable nitrogen atom, from R²¹, —N(R²¹)₂, —C(O)R²¹, —CO₂R²¹, —C(O)—C(O)R²¹, —C(O)CH₂C(O)R²¹, —SO₂R²¹, —SO₂N(R²¹)₂, —C(═S)N(R²¹)₂, —C(═NH)N(R²¹)₂ and —NR²¹SO₂R²¹;
wherein each recitation of optionally substituted alkyl refers to one or more substituents independently selected from halogen, —NO₂, —CN, —R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂, ═O, ═S, ═C(R²¹)₂, =N—N(R²³)₂, =N—OR²¹, ═N—NHC(O)R²¹, =N—NHCO₂R²², =N—NHSO₂R²² and =N—R²¹;
wherein each recitation of optionally substituted heterocyclyl refers to one or more substituents on an unsaturated carbon atom independently selected from halogen, —NO₂, —CN, —R²¹, —C(R²¹)═C(R²¹)₂, —C≡C—R²¹, OR²¹, —SR²², —S(O)R²², —SO₂R²², —SO₂N(R²³)₂, —N(R²³)₂, —NR²³C(O)R²¹, —NR²³C(O)N(R²³)₂, —NR²³CO₂R²², —OCO₂R²¹, —OC(O)N(R²³)₂, —OC(O)R²¹, —CO₂R²¹, —C(O)—C(O)R²¹, —C(O)R²¹, —C(O)N(R²³)₂, —C(═NR²³)N(R²³)₂, —C(═NR²³)OR²¹, —N(R²³)—N(R²³)₂, —N(R²³)C(═NR²³)—N(R²³)₂, —NR²³SO₂R²², —NR²³SO₂N(R²³)₂, —P(O)(R²¹)₂, —P(O)(OR²¹)₂, —OP(O)OR²¹ and —P(O)(NR²³)N(R²³)₂, ═O, ═S, ═C(R²¹)₂, =N—N(R²³)₂, =N—OR²¹, ═N—NHC(O)R²¹, ═N—NHCO₂R²², =N—NHSO₂R²² and =N—R²¹; or on a substitutable nitrogen atom, from R²¹, —N(R²¹)₂, —C(O)R²¹, —CO₂R²¹, —C(O)—C(O)R²¹, —C(O)CH₂C(O)R²¹, —SO₂R²¹, —SO₂N(R²¹)₂, —C(═S)N(R²¹)₂, —C(═NH)N(R²¹)₂ and —NR²¹SO₂R²¹;
wherein
each R²¹ is independently H, C₁₋₁₀alkyl, C₆₋₁₀aryl, C₅₋₁₀heteroaryl or C₃₋₁₀heterocyclyl;
each R²² is independently C₁₋₁₀alkyl or C₆₋₁₀aryl;
each R²³ is independently H, C₁₋₁₀alkyl, C₆₋₁₀aryl, C₅₋₁₀heteroaryl or C₃₋₁₀heterocyclyl, or two R²³ on the same nitrogen atom are taken together with the nitrogen atom to form a 5- to 8-membered ring having zero to two additional heteroatoms selected from N, O and S;
and m is 1, 2 or 3.

62. The method of any one of claims 1 to 58, wherein the NAE inhibitor is a compound listed in Table 1, or a salt, solvate and/or prodrug thereof, or a combination thereof.

63. A composition comprising an NAE inhibitor, or a salt, solvate and/or prodrug thereof, and a virus or genetic material encoding components of a virus.
